SEQUENCE LISTING (78 sequences)

SEQ ID NO: 1 - protein, 121 aa, synthetic construct
GSEDSNFPRTVMVNLNIHNRNTNTNPKRSSDYYDRSTSPWNLHRNEDPERYPSVIWEAKCRHLGCINADGNVDYHMNSVPIQQEILVLRREPPHSPNSFRLEKILVSVGCTCVTPIVHHVA

SEQ ID NO: 2 - protein, 130 aa, synthetic construct
GSEGETFFQKPESCPPVPGGSMKLDIGIINENQRVSMSRNIESRSTSPWDYTVTWDPNRYPSEVVQAQCRNLGCINAQGKEDISMNSVPIQQETLVVRRKHQGCSVSFQLEKVLVTVGCTCVTPVIHHVQ

SEQ ID NO: 3 - protein, 121 aa, synthetic construct
GSEDSNFPRTVMVNLNIHNRNTNTNPKRSSDYYDRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 4 - protein, 130 aa, synthetic construct
GSEGETFFQKPESCPPVPGGSMKLDIGIINENQRVSMSRNIESRSTSPWDYTVTWDPNRYPSEVVQAQCRNLGCINAQGKEDISMNSVPIQQETLVVRRKHQGSSVSFQLEKVLVTVGCTCVTPVIHHVQ

SEQ ID NO: 5 - protein, 123 aa, synthetic construct
QVQLVESGGGLVQAGGSLRLSCAASGLTASSYAMGWFRQAPGKEREFVAAISWISGGTKYADSVKGRFTMSRDNAKKQVYLEMNSLKPEDTAVYYCNDMPYGLDTRMDEYAYWGQGTQVTVSS

SEQ ID NO: 6 - protein, 11 aa, synthetic construct
AASGLTASSYA

SEQ ID NO: 7 - protein, 10 aa, synthetic construct
AISWISGGTK

SEQ ID NO: 8 - protein, 17 aa, synthetic construct
NDMPYGLDTRMDEYAYW

SEQ ID NO: 9 - protein, 123 aa, synthetic construct
QVQLVESGGGLVQAGGSLRLSCAASGLTGSSYAMGWFRQAPGKEREFVAAISWISGSTKYTDSVKDRFTMSRDNAKKQVYLEMNSLKPEDTAVYYCNDMPYGLDTRMDEYVYWGQGTQVTVSS

SEQ ID NO: 10 - protein, 10 aa, synthetic construct
AISWISGSTK

SEQ ID NO: 11 - protein, 17 aa, synthetic construct
NDMPYGLDTRMDEYVYW

SEQ ID NO: 12 - protein, 123 aa, synthetic construct
QVQLVESGGGLVQAGGSLRLSCAASGLTGSSYAMGWFRQAPGKEREFVAAISWISGGTKYTDSVKDRFTMSRDYAKKQVYLEMNSLKPEDTAVYYCNDMPYGLDTRMDEYVYWGQGTQVTVSS

SEQ ID NO: 13 - protein, 123 aa, synthetic construct
QVQLVESGGDLVQAGGSLRLSCAASGLTGSSYAMGWFRQAPGKEREFVAAISWISGDTKYTDSVKGRFTMSRDNAKKQVYLEMNSLKPEDTAVYYCNDMPYGLDTRMDEYVYWGQGTQVTVSS

SEQ ID NO: 14 - protein, 10 aa, synthetic construct
AISWISGDTK

SEQ ID NO: 15 - protein, 123 aa, synthetic construct
QVQLVESGGGLVQVGGSLRLSCEVSGLPVSSYAMGWFRQAPEKEREFVAAISWIGGDSRYAHFVKGRFTISRDNAKNTVHLQMDSLKPEDTAVYYCNDMPYGLDTRMDEYEYWGLGTQVTVSS

SEQ ID NO: 16 - protein, 11 aa, synthetic construct
EVSGLPVSSYA

SEQ ID NO: 17 - protein, 10 aa, synthetic construct
AISWIGGDSR

SEQ ID NO: 18 - protein, 17 aa, synthetic construct
NDMPYGLDTRMDEYEYW

SEQ ID NO: 19 - protein, 123 aa, synthetic construct
QVQLVESGGGLVQVGGSLRLSCEVSGLPVSSYAMGWFRQAPEKEREFVAAISWIGGDSRYAHFVKGRFTISRDNAKNTVHLQMDSLKPEDTAVYYCNDMPYGLDTRMDEYEYWGQGTQVTVSS

SEQ ID NO: 20 - protein, 123 aa, synthetic construct
QVQLVESGGGLVQPGGSLRLSCAVSGRTFSSYAMGWFRRPPGKEREFVAAISWSGGDTKYADFVTDRFTISRDDAKSTVYLQMNSLKPEDTAVYYCNDMPYGLDTRMDEYEYWGQGTQVTVSS

SEQ ID NO: 21 - protein, 11 aa, synthetic construct
AVSGRTFSSYA

SEQ ID NO: 22 - protein, 10 aa, synthetic construct
AISWSGGDTK

SEQ ID NO: 23 - protein, 123 aa, synthetic construct
QVQLVESGGGLVQAGGSLNLSCVISRRTPSGYAMGWFRQASGKEREFVAAISWIGGGAVYADSVKGRFTMSRDNAKNTVYLQMNSLKPEDTAVYYCNDMPYGLDTTMDDYDYWGQGTLVTVSS

SEQ ID NO: 24 - protein, 11 aa, synthetic construct
VISRRTPSGYA

SEQ ID NO: 25 - protein, 10 aa, synthetic construct
AISWIGGGAV

SEQ ID NO: 26 - protein, 17 aa, synthetic construct
NDMPYGLDTTMDDYDYW

SEQ ID NO: 27 - protein, 123 aa, synthetic construct
QVQLVESGGGLVQAGGSLNLSCVISRRTPSGYAMGWFRQASGKEREFVAAISWIGGGAVYADSVKGRFTMSRDNAKNTVYLQMNSLKPEDTAVYYCNDMPYGLDTTMDDYDYWGQGTQVTVSS

SEQ ID NO: 28 - protein, 123 aa, synthetic construct
QVQLVESGGDLVQAGGSLRLSCSVSGLRVSSYAMGWFRQAPGKEREFVAAISWVGGDTRYADSVKGRFTMSRDNAKKQVYLEMNSLKPEDTAVYYCNDMPYGMETRMDDYASWGQGTQVTVSS

SEQ ID NO: 29 - protein, 11 aa, synthetic construct
SVSGLRVSSYA

SEQ ID NO: 30 - protein, 10 aa, synthetic construct
AISWVGGDTR

SEQ ID NO: 31 - protein, 17 aa, synthetic construct
NDMPYGMETRMDDYASW

SEQ ID NO: 32 - protein, 123 aa, synthetic construct
QVQLVESGGGLVQAGGSLRLSCAASGLAVSSYAMGWFRQAPGKEREFVAAISWISGGTKYADSVKDRFTMSRDNAKKQVYLEMNSLKPEDTAVYYCNDMPYGLDTRMDDYKYWGQGIQVTVSS

SEQ ID NO: 33 - protein, 11 aa, synthetic construct
AASGLAVSSYA

SEQ ID NO: 34 - protein, 17 aa, synthetic construct
NDMPYGLDTRMDDYKYW

SEQ ID NO: 35 - protein, 116 aa, synthetic construct
QVQLVESGGGLVNTGGSLRLSCVVSGHTSEISAMGWYRQAPGKQRELVALITSRSGDTHYSDSVKGRFSISRDNAKNTMYLQMNSLNPDDTAVYYCVHNEPGHLYMAQGTQVTVSS

SEQ ID NO: 36 - protein, 11 aa, synthetic construct
VVSGHTSEISA

SEQ ID NO: 37 - protein, 10 aa, synthetic construct
LITSRSGDTH

SEQ ID NO: 38 - protein, 10 aa, synthetic construct
VHNEPGHLYM

SEQ ID NO: 39 - protein, 116 aa, synthetic construct
QVQLVESGGGLVKAGGSLRLSCVVSGHTSEISAMGWYRQAPGKQRELVALITSRSGDTHYSDSVKGRFSISRDNAKNTMYLQMNSLNPDDTAVYYCVHNEPGHLYMAQGTQVTVSS

SEQ ID NO: 40 - protein, 116 aa, synthetic construct
QVQLVESGGGLVRTGGSLRLSCSVSGDISSQSAMGWYRQVPGRQRELVAMIHTTKGDTYYADSVKGRFTISRINANNTIYLQMNNLKPEDTAIYYCTHNEPGDLYMGQGTQVTVSS

SEQ ID NO: 41 - protein, 11 aa, synthetic construct
SVSGDISSQSA

SEQ ID NO: 42 - protein, 10 aa, synthetic construct
MIHTTKGDTY

SEQ ID NO: 43 - protein, 10 aa, synthetic construct
THNEPGDLYM

SEQ ID NO: 44 - protein, 116 aa, synthetic construct
QVQLVQSGGGLVQTGGSLRLSCSVSGDISSQSAMGWYRQVPGRQRELVAMIHTTKGDTYYADSVKDRFTISRINANNTIHLQMNNLKPEDTAIYYCTHNEPGDLYMGQGTQVTVSS

SEQ ID NO: 45 - protein, 116 aa, synthetic construct
QVQLVESGGGLVRTGGSLRLSCSVSGDISSQSAMGWYRQVPGRQRELVAMIHTTKGDTYYADSVKGRFTISRINANNTIYLQMNNLKPEDTAIYYCTHNEPGDLYMGQGTLVTVSS

SEQ ID NO: 46 - protein, 12 aa, synthetic construct
SVSGDISSQSAM

SEQ ID NO: 47 - protein, 116 aa, synthetic construct
EVQLVESGGGSVQAGDSLRLSCAASGRANSINWFGWFRQTPGKEREFVAGIRWSDAYTEYANSVKGRFTISRDNAKNTVDLQMDSLKPEDTAVYYCVLDLSTVRYWGQGTQVTVSS

SEQ ID NO: 48 - protein, 122 aa, synthetic construct
EVQLVESGGGLVQAGGSLRLSCAASGRTYDAMGWLRQAPGKEREFVAAISGSGDDTYYADSVKGRFTISKDNAGITMYLQMNSLKPEDTAVYYCATRRGLYYVWDSNDYENWGQGTQVTVSS

SEQ ID NO: 49 - protein, 125 aa, synthetic construct
QVQLVESGGGLVQPGGSLRLSCVASGFTFSSFAMSWYRQDPGKERKLVAAITNDGGTTFYSDLVKGRFTISRDNTKNTVYLQMNNVKPDDAAVYYCMAVRGLYGSNWYDYPFELWGQGTQVTVSS

SEQ ID NO: 50 - protein, 11 aa, synthetic construct
VASGFTFSSFA

SEQ ID NO: 51 - protein, 10 aa, synthetic construct
AITNDGGTTF

SEQ ID NO: 52 - protein, 19 aa, synthetic construct
MAVRGLYGSNWYDYPFELW

SEQ ID NO: 53 - protein, 125 aa, synthetic construct
QVQLVESGGGLVQPGGSLRLSCVASGFTFSSFAMSWYRQDPGRERKLVAAITNDGGTTFYSDLVKGRFTISRDNTKNTVYLQMNNVKPDDAAVYYCMAVRGLYGSNWYDYPFELWGQGTQVTVSS

SEQ ID NO: 54 - protein, 116 aa, synthetic construct
QVQLVESGGGFVQAGGSLKLSCSVSGNTFITNHMGWFRQPPGKGREFVAGIRWSDGSTFYGDSVRGRFTISGDNAKHTLYLQMNSLKPDDTAVYYCYIDSGSDRYYGQGTLVTVSS

SEQ ID NO: 55 - protein, 11 aa, synthetic construct
SVSGNTFITNH

SEQ ID NO: 56 - protein, 10 aa, synthetic construct
GIRWSDGSTF

SEQ ID NO: 57 - protein, 10 aa, synthetic construct
YIDSGSDRYY

SEQ ID NO: 58 - protein, 11 aa, synthetic construct
AASGRANSINW

SEQ ID NO: 59 - protein, 10 aa, synthetic construct
GIRWSDAYTE

SEQ ID NO: 60 - protein, 10 aa, synthetic construct
VLDLSTVRYW

SEQ ID NO: 61 - protein, 9 aa, synthetic construct
AASGRTYDA

SEQ ID NO: 62 - protein, 10 aa, synthetic construct
AISGSGDDTY

SEQ ID NO: 63 - protein, 18 aa, synthetic construct
ATRRGLYYVWDSNDYENW

SEQ ID NO: 64 - protein, 128 aa, synthetic construct
QVQLVESGGGLVQPGGSLRLSCAISGITLDYYAVGWFLQAPGKEREGISCMRNWDGRTVYAPSVKGRFTISSDNAKKMVYLEMDNLKSEDTGVYYCAAGPLPPGISCRIPTPLGYDDWGQGTQVTVSS

SEQ ID NO: 65 - protein, 134 aa, synthetic construct
QVQLVESGGDLVQPGGSLRLSCAASGFTLDDYVIGWFRQAPGKEREGVACISSISNSDGRAYYVDSVKGRFTISRDNAKNTVYLQMNSLKLEDTAVYYCAAERGRIDPDVAGYFLRESYVMDYWGNGTLVTVSS

SEQ ID NO: 66 - protein, 126 aa, synthetic construct
QVQLVESGGGLVQPGGSLRLSCAASGFIFSHYWMYWVRQAPGKGLEWVSGINTGGRTTVYADSVRGRFTTSRDNAKNTLYLQMTSLQSEDTAVYYCTALRGDYGKDLTSLDEYEYWGQGTQVTVSS

SEQ ID NO: 67 - protein, 126 aa, synthetic construct
QVQLVESGGGLVQPGGSLRLSCAASGFIFSHYWMYWVRQAPGKGLEWVSAINTGGRNTVYADSVKGRFTISRDNAKNTLYLQMNSLKSEDTAVYYCTALRGDYGKDLVSLDEFEYWGQGTQVTVSS

SEQ ID NO: 68 - protein, 11 aa, synthetic construct
AASGFTLDDYV

SEQ ID NO: 69 - protein, 13 aa, synthetic construct
CISSISNSDGRAY

SEQ ID NO: 70 - protein, 25 aa, synthetic construct
AAERGRIDPDVAGYFLRESYVMDYW

SEQ ID NO: 71 - protein, 11 aa, synthetic construct
AASGFIFSHYW

SEQ ID NO: 72 - protein, 10 aa, synthetic construct
GINTGGRTTV

SEQ ID NO: 73 - protein, 20 aa, synthetic construct
TALRGDYGKDLTSLDEYEYW

SEQ ID NO: 74 - protein, 11 aa, synthetic construct
AASGFIFSHYW

SEQ ID NO: 75 - protein, 10 aa, synthetic construct
AINTGGRNTV

SEQ ID NO: 76 - protein, 20 aa, synthetic construct
TALRGDYGKDLVSLDEFEYW

SEQ ID NO: 77 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 78 - protein, 133 aa, Homo sapiens [sequence omitted]

(19) United States
(12) Patent Application Publication
(10) Pub. No.: US 2026/0209334 A1
(43) Pub. Date: Jul. 23, 2026

(54) THERAPEUTIC VARIABLE DOMAIN OF HEAVY CHAIN (VHH) ANTIBODIES CROSS-NEUTRALIZING INTERLEUKIN 17 (IL-17) POLYPEPTIDES

(71) Applicants: MAX-PLANCK-GESELLSCHAFT ZUR FÖRDERUNG DER WISSENSCHAFTEN E.V., Munich (DE); GEORG-AUGUST-UNIVERSITÄT GÖTTINGEN STIFTUNG ÖFFENTLICHEN RECHTS, UNIVERSITÄTSMEDIZIN, Göttingen (DE)

(72) Inventors: Dirk GÖRLICH, Göttingen (DE); Waltraud TAXER, Göttingen (DE); Karen LINNEMANNSTOENS, Göttingen (DE); Bianka MUSSIL, Göttingen (DE); Kathrin GREGOR, Göttingen (DE); Renate REES, Göttingen (DE); Metin AKSU, Göttingen (DE); Matthias DOBBELSTEIN, Göttingen (DE)

(73) Assignees: MAX-PLANCK-GESELLSCHAFT ZUR FÖRDERUNG DER WISSENSCHAFTEN E.V., Munich (DE); GEORG-AUGUST-UNIVERSITÄT GÖTTINGEN STIFTUNG ÖFFENTLICHEN RECHTS, UNIVERSITÄTSMEDIZIN, Göttingen (DE)

(21) Appl. No.: 19/140,680

(22) PCT Filed: Dec. 27, 2023
(86) PCT No.: PCT/EP2023/087865

§ 371 Date: Jun. 18, 2025

(30) Foreign Application Priority Data
Dec. 28, 2022 (EP) 22216931.0

(51) Int. Cl. C07K 16/24; A61P 17/06

(52) U.S. Cl. CPC C07K 16/244; A61P 17/06; C07K 2317/569

(57) ABSTRACT

The present invention provides VHH antibodies that neutralize the pro-inflammatory interleukin IL-17 in its IL-17A and IL-17F homodimeric as well as in its IL-17AF heterodimeric forms. Pharmaceutical compositions comprising the VHH antibodies and uses for treatment of inflammatory and immune-related diseases and disorders are also provided.

### FIELD OF THE INVENTION

The present invention pertains in the fields of antibody technology, medicine, pharmacology, infection biology, and medical diagnostics. More specifically, the present disclosure provides VHH antibodies that neutralize the pro-inflammatory interleukin 17 (IL-17) in its IL-17A and IL-17F homodimeric as well as in its IL-17AF heterodimeric forms.

### BACKGROUND OF THE INVENTION

Interleukin 17 (IL-17) is a multifunctional cytokine. It is produced by a specific subclass of CD4-positive T helper cells, i.e., Th17 cells, but also by other CD4- or CD8-positive T cells and gamma/delta T cells. A major effect of IL-17 consists of the attraction of neutrophils and monocytes, and the production of additional cytokines and chemokines by its target cells. Taken together, IL-17 is a key mediator of inflammatory responses. These responses can be of strong benefit to a patient, e.g., when building a defense against various types of infections (Mills, 2022). However, excessive or uncontrolled production of IL-17 can also provoke or exacerbate autoimmune diseases such as psoriasis, rheumatoid arthritis, asthma, or inflammatory bowel diseases.

Upstream signaling pathways tightly control the synthesis and secretion of IL-17 upon mucosal infections. Most notably, Interleukin 23 (IL-23) and IL-1 beta, released from activated dendritic cells or macrophages, stimulate Th17 cells to release IL-17.

Target cells of IL-17 can vary since the receptors are found in a wide array of human cell types. Accordingly, the response can be heterogeneous, too. Most importantly, however, IL-17-exposed epithelial cells release chemokines such as CXCL1 and CXCL8, thereby attracting neutrophilic granulocytes and macrophages to maintain the homeostasis of the epithelial barrier.

In autoimmune diseases, elevated IL-17 levels at the site of inflammation activate the release of Interleukin-6, Tumor Necrosis Factor Alpha (TNF-alpha), and matrix metalloproteinases (MMPs). This leads to tissue damage and amplifies the inflammatory response (Mills, 2022). IL-17 proteins form dimers. They comprise a family of currently six members, IL-17A through IL-17F. Of these, IL-17A and IL-17F are considered the major and most relevant ones; they are also the best-studied ones. They bind to five receptors, IL-17RA-E, forming heterodimeric receptor complexes. IL-17A and IL-17F bind and activate the IL-17RA and IL-17RC receptors as homodimers or as a heterodimer IL-17A/F (Nies and Panzer, 2020). IL-17 binding requires the adaptor protein TRAF3IP2 and can lead to the lateral assembly of the receptors, at least partially explaining the onset of intracellular signaling, such as the activation of NF-kappa-B and Mitogen-Activated Protein (MAP) kinases (Wilson et al., 2022).

A number of approaches were developed to interfere with the production and/or activities of IL-17. In particular, monoclonal antibodies can target the upstream IL-23 but, most importantly, IL-17 itself or its receptor. The neutralization of IL-17 then dampens the inflammatory response, which is of benefit when treating autoimmune diseases.

Antagonists to IL-17, particularly antibodies, were successfully applied to treat a number of diseases, giving rise to FDA-approved drugs. Most notably, inflammatory skin diseases, i.e., psoriasis and psoriatic arthritis, as well as Hidradenitis suppurativa (“inverse acne”), were treated successfully by IL-17 neutralizing antibodies (summarized in Skroza et al., 2017; Mills, 2022). Therapeutics include Ixekizumab and Secukinumab that target IL-17A alone in plaque psoriasis (Langley et al., 2014) and psoriatic arthritis (Mease et al., 2015) as well as Bimekizumab against both IL-17A and IL-17F in plaque psoriasis (Reich et al., 2021; Warren et al., 2021) and psoriatic arthritis (Glatt et al., 2018).

However, such antibodies are typically applied systemically, with little or no spatial restriction to the actual sites of inflammation. It should be noted in this context that such indiscriminate block of IL-17 can also exacerbate bacterial or fungal infections as, e.g., reported for Bimekizumab (Reich et al., 2021; Warren et al., 2021). Ideally, the neutralization of IL-17, when treating autoimmune diseases, should be locally confined to the site of the disease rather than applied to the whole body. With a more compact, thermostable, and versatile antibody version, topical applications may become feasible.

As IL-17 plays a major role in the development of plaque psoriasis it is the molecular target of several recently launched biological treatments, mainly monoclonal antibodies (mAbs), such as Cosentyx (Novartis) and Taltz (Lilly), both of which target IL-17A. Research shows that targeting IL-17F isoform in addition to IL-17A provides higher efficacy in treating plaque psoriasis. The recently launched Bimzelx (UCB) was the first mAb targeting both IL-17A and IL-17F to be approved by the European Medicines Agency (EMA). MoonLake Immunotherapeutics developing a nanobody targeting both IL-17A and IL-17F reported superior results vs. Cosentyx in a phase 2 plaque psoriasis clinical trial. All the above-mentioned antibodies, indicated only for moderate to severe psoriasis patients, are administered by subcutaneous injection for systemic drug distribution and carry risk of considerable side effects. These drugs are also expensive as they require chronic, life long, bi-weekly injections, each at a cost of several thousand dollars. Mild psoriasis, which accounts for 50% of plaque psoriatic patients, unfortunately has no safe and affordable biological drug available. Experience shows that even the 28% of patients with moderate plaque psoriasis tend to avoid or delay onset of these biological treatments due to the associated risks.

Nanobodies (VHH antibodies) comprise a novel class of therapeutic proteins based on monovalent, camelid-derived heavy-chain-only antibodies. In contrast to monoclonal IgGs, which are manufactured in mammalian cells, nanobodies can be produced in bacteria or yeast. Apart from their low molecular weight, certain nanobodies proved to be hyper-thermostable and displayed a particularly high affinity (Güttler et al., 2021).

One IL-17 antagonist, Sonelokimab, was engineered based on single-chain antibodies/nanobodies. It is a trimer of nanobodies targeting IL-17A, IL-17F, and albumin. The idea is to absorb both versions of IL-17 and increase the half-life of the antagonist by binding to albumin. However, this compound, based on its size and albumin-binding entity, is still suitable for systemic rather than topical application. Similar to the classical antibody treatments, the study participants had an increased incidence of fungal Candida infections (Papp et al., 2021).

WO 2012/156219 discloses single domain antibodies that specifically bind to human IL-17A, human IL-17F and/or human IL-17A/F, and tandem fusions of them, though the affinity of the individual VHHs to IL-17F isoform was rather weak.

On top of skin diseases, other inflammatory disorders might become amenable to treatment with IL-17 antagonists. These include but are not limited to ankylosing spondylitis (with approved application), but also Multiple Sclerosis, Rheumatoid arthritis, Asthma, Graft-versus-Host Disease, and even diseases like Alzheimer's′, Fatty Liver Disease, and COVID-19 (all clinical studies ongoing as reviewed in Mills, 2022). Future applications may consist of the treatment of autism, Parkinson's Disease, atherosclerosis, stroke, and sepsis. Most of these applications will benefit from a more versatile type of antibody with high affinity, stability, and versatility regarding application routes and fusion to additional/stabilizing entities. This illustrates the immense spectrum of benefits expected from VHH antibodies targeting IL-17A/F.

There is a clear unmet need for novel immunotherapeutic agents that will target both IL-17A and IL-17F and that will be safely used to treat inflammatory, immune, and autoimmune disorders and diseases. Such agents may advantageously be used to treat mild and moderate plaque psoriasis by local administration.

### SUMMARY OF THE INVENTION

The present invention provides compositions and methods for the amelioration of symptoms associated with overactivity of the cytokine IL-17 and provides novel classes of single-domain VHH antibodies that neutralize the human cytokine for treating patients with inflammatory and/or immune-related disorders caused by and/or associated with an overactivity of this cytokine. The VHH antibodies of the present invention are provided for the treatment of immune, autoimmune, and inflammatory diseases and disorders, including skin diseases and disorders.

The novel VHH antibodies of the present invention are categorized in four classes based not only on sequence but also on data gained from crystal and modelled structures of complexes of the novel VHH antibodies and human IL-17 isoforms. These crystal and modelled structures have been used to define the amino acid residues in the VHH sequence that are interacting with IL-17 isoforms and mostly contribute to binding and blocking their binding to a human IL-17 receptors therefore preventing or inhibiting receptor activation.

In certain embodiments, these VHH antibodies are in monovalent form, e.g., as a single VHH domain or as a fusion to a heterologous protein such as serum albumin useable in a wide range of formats. In certain embodiments, The VHH antibodies are in multivalent form, e.g., as bivalent Fc-fusion. The use in a monovalent format is possible due to their very high affinity. In certain embodiments, the VHH antibodies have, in the monovalent format, a target affinity in picomolar or even low picomolar. In certain embodiments, the VHH antibodies neutralizes all major IL-17 isoforms, particularly human IL-17A homodimers, human IL-17F-homodimers and human IL-17A/F heterodimers with similar high potency.

The present invention is based in part on the finding that representative VHH antibodies show unexpectedly cross reaction between IL-17A and IL-17F isoforms, with sub-nanomolar affinities. The present invention is further based on the results of preclinical studies of some of the anti-IL-17 VHH antibodies for treatment of plaque psoriasis, suggesting the therapeutic potential to relieve symptoms of this disease.

The VHH antibodies of the present invention are designed, according to some embodiments, to be administered locally to the dermis and to be eliminated in a way that should prevent systemic side effects. The results of an ex-vivo study with VHH antibodies of the present invention suggest the potential for a highly efficacious, specific, yet safer and more convenient treatment for the large and underserved population of mild to moderate plaque psoriasis patients.

The present invention provides a VHH antibody recognizing a human IL-17 polypeptide, which cross-reacts with a plurality of different human IL-17 polypeptides comprising (i) a human IL-17A homodimer, (ii) a human IL-17F homodimer and (iii) a human IL-17A/F heterodimer and prevents or inhibits activation of a human IL-17 receptor. The VHH antibodies of the present invention bind with extreme affinity to different IL-17 isoforms that differ in their epitopes.

According to some embodiments, the binding affinity of a monomeric VHH, expressed as a dissociation constant KD, to an immobilized human IL-17A or IL-17F homodimer is 5 nM, 1 nM, 500 pM, 300 pM, 100 pM, 50 pM, or less.

The present invention provides according to one aspect, a VHH antibody that binds to and neutralizes IL-17A, IL-17F and IL17AF binding to and activation of a human IL-17 receptor, comprising an amino acid sequence selected from SEQ ID 19, 15, 5, 23, 12, 13, 20, 27, 28, 32, 35, 39, 40, 44, 45, 49, 53, and 54, or a variant thereof having at least 80% identity with any of these sequences.

According to some embodiments, the invention provides a VHH antibody that binds to and neutralizes IL-17A, IL-17F and IL17AF, comprising an amino acid sequence selected from the group consisting of SEQ ID NO. 19, 15, 39, 35, 5, 23, 12, 13, 20, 27, 28, 32, 40, 44, 45, 49, 53, and 54, or a variant thereof having at least 85% identity with any of these sequences.

According to some embodiments, the invention provides a VHH antibody binds to and neutralizes IL-17A, IL-17F and IL17AF, comprising an amino acid sequence selected from the group consisting of SEQ ID NO. 19, 15, 39, 35, 5, 23, 12, 13, 20, 27, 28, 32, 40, 44, 45, 49, 53, and 54, or a variant thereof having at least 90% identity with any of these sequences.

According to some embodiments, the VHH antibody that binds to IL-17A, IL-17F and IL17AF and neutralizes their activity, comprises an amino acid sequence selected from SEQ ID NO. 19, 15, 5, 23, 12, 13, 20, 27, 28, and 32 (Class A); SEQ ID NO. 39, 35, 40, 44, and 45 (Class B), SEQ ID NO. 49, and 53 (Class C), and SEQ ID NO. 54 (Class D), or a variant thereof having at least 80% identity with any of these sequences.

According to some embodiments, the variant has at least 91%, 92%, 93%, 94% or 95% sequence identity to the VHH antibody. According to more particular embodiments, the variant has at least 95%, 96%, 97%, 98% or 99% sequence identity to the VHH antibody.

Variants comprising substitutions in 1-10 amino acid residues are also included in the scope of the present invention. The substitutions may be selected from conservative substitutions, non-conservative substitutions, and combinations thereof.

According to some embodiments, a variant of a VHH antibody described above, wherein 1, 2, 3, 4, 5, 6, 7, 8, 9, 10 or 11 amino acid are substituted, deleted, or added is provided. According to particular embodiments, 1-5 amino acids in a VHH antibody are substituted, deleted, or added. According to some specific embodiments, the substitution, deletion, or addition retains or improves at least one physical property of the VHH antibody. According to some embodiments a substitution or a combination of 2-5 substitutions, additions or deletions improve the stability and/or producibility of a specific VHH antibody according to the present invention.

According to some embodiments, a substitution of the non-CDR residue 115 of the VHH antibody Re42B04 from Leu (Leu) to Gln (Q) improves the physical properties of the resultant VHH antibody Re42B04a.

In particular embodiments, at least one residue in an N-glycosylation site is substituted or deleted to prevent potential glycosylation of the VHH antibody. According to particular embodiments, a residue selected from Asparagine (Asn, N), Threonine (Thr, T), and Serine (Ser, S) is substituted or deleted.

Variants comprising 1-3 amino acid substitutions in one, two or even three CDR sequences are also included in the scope of the present invention. According to some embodiments, the substitution is a conservative substitution. According to other embodiments, the substitution is a non-conservative substitution.

The present invention provides a VHH antibody binds to and neutralize the binding of a human IL-17A, IL-17F and IL17AF dimer to a human IL-17 receptor, the VHH antibody belongs to a class of structurally-related antibodies, wherein the class is selected from:
i. Class A comprising a CDR3 sequence of the formula NDMPYGX₁X₂TX₃MDX₄YX₅X₆W wherein, X₁ is selected from L and M, X₂ is selected from D and E, X₃ is selected from R and T, X₄ is selected from E and D, X₅ is selected from A, V, ED and K and X₆ is selected from Y and S;
ii. Class B comprising a CDR3 sequence of the formula X₁HNEPGX₂LYM wherein, X₁ is selected from V and T and X₂ is selected from H and D;
iii. Class C comprising the CDR3 sequence MAVRGLYGSNWYDYPFELW (SEQ ID NO. 52), and
iv. Class D comprising the CDR3 sequence YIDSGSDRYY (SEQ ID NO. 57),
wherein the sequence identity between different VHH antibodies in a specific class is 80% or more.

According to some embodiments, the VHH of Class A comprised a CDR3 having a sequence selected from SEQ ID NO. 8, 11, 18, 26, 31 and 34, a CDR2 having a sequence selected from SEQ ID NO. 7, 14, 17, 22, 25 and 30, and a CDR1 having a sequence selected from SEQ ID NO. 6, 10, 16, 21, 24, 29 and 33.

According to some embodiments, the VHH antibody or variant thereof comprises a CDR3 sequence as shown in any one of SEQ ID Nos: 8, 11, 18, 26, 31, 31, 38, 43, 52 and 57 or a CDR3 sequence, or a sequence which has an identity of at least 80%, at least 90% or at least 95% to the CDR3 sequence.

According to some specific embodiments, a VHH antibody of Class A that recognizes and neutralizes IL-17 is provided, wherein the VHH antibody comprises a set of 3 CDR sequences, the set is selected from: SEQ ID NO. 16, 17 and 18; SEQ ID NO. 6, 7 and 8; SEQ ID NO. 10, 7 and 11; SEQ ID NO. 10, 14 and 11; SEQ ID NO. 21, 22 and 18; SEQ ID NO. 24, 25 and 26; SEQ ID NO. 29, 30 and 31; SEQ ID NO. 33, 7 and 34.According to some specific embodiments, a VHH antibody of Class B that recognizes and neutralizes IL-17 is provided, wherein the VHH antibody comprises a set of 3 CDR sequences, the set is selected from: SEQ ID NO. 36, 37, and 38; SEQ ID NO. 41, 42 and 43; and SEQ ID NO. 46, 42 and 43.

According to some specific embodiments, a VHH antibody of Class C that recognizes and neutralizes IL-17 is provided, wherein the VHH antibody comprises a set of 3 CDR sequences, the set comprises SEQ ID NO. 50, 51 and 52.

According to some specific embodiments, a VHH antibody of Class D that recognizes and neutralizes IL-17 is provided, wherein the VHH antibody comprises a set of 3 CDR sequences, the set comprises SEQ ID NO. 56, 57 and 58.

According to some particular embodiments, a VHH antibody of Class A or Class B is provided comprising a set of 3 CDR sequences, wherein the set is selected from: SEQ ID NO. 16, 17 and 18, and SEQ ID NO. 36, 37, and 38.

According to some embodiments, the VHH antibody is selected from RE42B04a (SEQ ID NO. 19), Re42B04 (SEQ ID NO. 15), Re42F08 (SEQ ID NO. 39), and Bm17B02 (SEQ ID NO. 35).

According to particular embodiments, the present invention provides the VHH antibody Re42B04a comprising a VHH sequence as shown in SEQ ID NO. 19, or a VHH antibody, which is a variant thereof having at least 90% identity.

According to particular embodiments, the present invention provides the VHH antibody Re42B04 comprising a VHH sequence as shown in SEQ ID NO. 15, or a VHH antibody, which is a variant thereof having at least 90% identity.

According to particular embodiments, the present invention provides the VHH antibody Bm17B02 comprising a VHH sequence as shown in SEQ ID NO. 35, or a VHH antibody, which is a variant thereof having at least 90% identity.

According to some embodiments, Class A VHH antibodies comprise the following positions that interact with IL-17A: position 1 that is Q; position 29 that is A, G V, F or P; position 30 that is S; position 31 that is S or G; position 32 that is Y; position 33 that is A; position 50 that is A; position 51 that is I; position 52 that is S; position 54 that is I, S, or V; position 55 that is S or G; position 57 that is G, S, or D; position 58 that is T, S or A; position 59 that is K, R or V; position 100 that is P; position 101 that is Y; position 103 that is L or M; position 104 that is D or E; position 106 that is R; and position 109 that is E or D.

According to some embodiments, Class A VHH antibodies comprise the following positions that interact with IL-17F: Position 29 that is A, G V, F or P; position 30 that is S; position 31 that is S or G; position 32 that is Y; position 33 that is A; position 50 that is A; position 51 that is I; position 52 that is S; position 54 that is S, I or V; position 55 that is S or G; position 57 that is G, S, or D; position 58 that is T, S or A; position 59 that is K, R or V; position 100 that is P; position 101 that is Y; position 103 that is L or M; position 104 that is D or E; position 106 that is R; and position 109 that is E or D.

According to some embodiments, Class B VHH antibodies comprise the following positions that interact with IL-17A: position 3 that is Q, position 31 that is I or Q, position 32 that is S, position 33 that is A, position 37 that is Y, position 45 that is R, position 52 that is H, position 59 that is H or Y, position 99 that is N, position 100 that is E, position 101 that is P, position 102 that is G, position 103 that is H or D, position 104 that is L, position 105 that is Y and position 106 that is M.

According to some embodiments, Class B VHH antibodies comprise the following positions that interact with IL-17F: position 3 that is Q, position 31 that is I or Q, position 32 that is S, position 33 that is A, position 37 that is Y, position 45 that is R, position 47 that is L, position 50 that is L or M, position 52 that is T or H, position 59 that is H or Y, position 99 that is N, position 100 that is E, position 101 that is P, position 102 that is G, position 103 that is H or D, position 104 that is L, position 105 that is Y and position 106 that is M.

A further aspect of the present invention relates to a set of two or more different VHH antibodies, wherein at least one VHH antibody is as described above.

In certain embodiments, the VHH antibody as described above is covalently or non-covalently conjugated to a heterologous moiety, which may be selected from a labeling group, a capture group or an effector group.

In certain embodiments, the VHH antibody as described above is fused to a heterologous polypeptide moiety, e.g., to an IgG Fc fragment, to serum albumin or to an albumin-binding moiety. In certain embodiments, the VHH antibody is conjugated to one or several polymer moieties, particularly hydrophilic polymer moieties, such as polyethylene glycol (PEG), to increase the molecular weight of the antibody conjugate and, thus, delay renal clearance. The molecular weight of a polymer moiety may vary over a broad range, for example in the range of about 5 kDa to about 80 kDa. Such coupling may be performed through e.g., amino or carboxyl groups already present in the VHHs (e.g., amino and carboxy terminals) and/or through the side chains of lysine, aspartic acid, glutamic acid or cysteine residues, or through engineered backbone or side chains of other amino acids, and involve known chemistries for forming amide bonds, secondary amine bonds, urea bonds or thioether bonds.

According to some embodiments, the VHH antibody neutralizes the binding of a human IL-17 dimer to a human IL-17 receptor. According to specific embodiments, the VHH antibody neutralizes the binding of (i) a human IL-17A homodimer, (ii) a human IL-17F homodimer and (iii) a human IL17A/F heterodimer to a human IL-17 receptor. According to more specific embodiments, the VHH antibody neutralizes the binding of at least one of (i) a human IL-17A homodimer, (ii) a human IL-17F homodimer and (iii) a human IL17A/F heterodimer, to a human IL-17 receptor at a concentration of about 10 nM or less, of about 3 nM or less, of about 1 nM or less, of about 0.3 nM or less, when tested in a cell-based assay under affinity-limited test conditions.

A VHH antibody that is a variant of the VHH antibodies disclosed above and competes for IL-17 binding with any of the VHH antibodies disclosed above is also within the scope of the present invention.

According to some embodiments, the VHH antibody is stable, particularly thermostable or hyperthermostable. According to some specific embodiments, the VHH antibody has a melting temperature of at least about 65° C., of at least about 80° C., of at least 90° C. or of at least about 95° C. when measured under non-reducing conditions.

According to some embodiments, the VHH antibody has an aggregation temperature of at least about 60° C., of at least 70° C., of at least about 80° C., of at least about 90° C., or of at least about 95° C., when measured under non-reducing conditions.

The VHH antibody of any one of the previous embodiments, which is non-glycosylated, or which is glycosylated.

According to some embodiments, the VHH antibody or variant thereof is in a monovalent format.

According to some embodiments, the VHH antibody or variant thereof is in a dimeric or multimeric format.

The resent invention also provides a set of two or more different VHH antibodies recognizing a human IL-17 polypeptide, particularly an IL-17A homodimer, an IL-17F homodimer and an IL17A/F heterodimer, comprising at least one VHH antibody or a variant thereof disclosed above, particularly a VHH antibody in a monovalent format.

A VHH antibody described above or a set of VHH antibodies are suitable for use in medicine, e.g., human medicine, particularly for use in therapy, e.g., in the prevention or treatment of a disorder caused by and/or associated with an overactivity of IL-17, particularly an overactivity of IL-17A and/or IL-17F, or in diagnostics, e.g., for detecting IL-17 in a patient sample, e.g., in a body fluid or tissue sample, or in research.

The present invention also provides, according to another aspect, a nucleic acid molecule encoding a VHH antibody or a subunit of a VHH antibody described above, preferably in operative linkage with a heterologous expression control sequence, or included in a vector.

The present invention also provides a recombinant cell or non-human organism transformed or transfected with a nucleic acid molecule or a vector.

According to some embodiments, the cell or organism is selected from a bacterium such as E. coli Bacillus sp., a unicellular eukaryotic organism, e.g., yeast such as Pichia pastoris, or Leishmania, an insect cell, a mammalian cell, and a plant cell.

According to some embodiments, the VHH antibody or variant thereof is produced in a bacterium, e.g., E. coli, or in a yeast, e.g., Pichia pastoris.

The invention also provides a method for recombinant production of a VHH antibody described above, comprising cultivating a cell or an organism in a suitable medium and obtaining the VHH antibody from the cell or organism or from the medium.

According to some embodiments, the method comprises cultivating cells from a bacterium, a yeast, an insect, a mammalian, and a plant. According to some embodiments, the cells are mammalian cells. According to some embodiments, the mammalian cells are Chinese Hamster Ovary (CHO) cells.

According to some embodiments, the method comprises cultivating a yeast such as Pichia pastoris and obtaining the VHH antibody from the medium.

The invention further provides, according to another aspect, a pharmaceutical composition comprising at least one VHH antibody defined above, and a pharmaceutically acceptable carrier, excipient, or diluent.

According to some embodiments, the pharmaceutical composition comprises a plurality of VHH antibodies described above, e.g., a set of specific VHH antibodies.

According to some embodiments, the formulation is for local administration. According to some embodiments, the formulation is for topical administration. Pharmaceutical compositions provided according to the present invention may be formulated as liquid, solid or semi-solid states. According to some embodiments, the pharmaceutical composition is formulated as a cream, paste, gel, hydrogel, ointment, lotion, and emulsion. According to other embodiments, the pharmaceutical composition is a liquid formulation. According to some embodiments, the pharmaceutical composition is formulated for parenteral administration, e.g., by injection or infusion. According to some embodiments, the pharmaceutical composition is formulated for intradermal injection.

According to some embodiments, the pharmaceutical composition is formulated as sustained release, slow release, or delayed release.

The invention further provides, according to another aspect, a diagnostic composition comprising at least one VHH antibody defined above, and an acceptable carrier, excipient or diluent.

Pharmaceutical and diagnostic kits comprising at least one VHH antibody and instructions for use are also provided.

The present invention also provides a pharmaceutical composition comprising at least one VHH antibody described above for use in medicine, particularly for use in therapy or diagnostics.

According to some embodiments, the pharmaceutical composition is for use in the prevention or treatment of a disorder caused by and/or associated with an overactivity of IL-17, particularly with an overactivity of IL-17A and/or IL-17F.

According to some embodiments, the pharmaceutical composition is for use in the prevention or treatment of an inflammatory and/or immune-related disorder.

According to some embodiments, the pharmaceutical composition is for use in the prevention or treatment of an inflammatory and/or immune-related skin disorder.

According to some embodiments, the pharmaceutical composition is for use in the prevention or treatment of asthma, psoriasis, arthritis, hidradenitis suppurativa, inflammatory bowel disease (Crohn's disease, ulcerative colitis), multiple sclerosis, skin cancer, ankylosing spondylitis, uveitis, atopic dermatitis, graft versus host disease, Alzheimer's disease, fatty liver disease, sepsis, ischemic stroke, Parkinson's disease, active non-radiographic axial spondylarthrites with objective clinical signs of inflammation, systemic lupus erythematosus (SLE), familial mediterranean fever (FMF), Tumor necrosis factor receptor-associated periodic syndrome (TRAPS), Hidradenitis Suppurative (HS), Pemphigus vulgaris (PV), Pityriasis rubra pilaris (PRP), Alopecia Areata, systemic sclerosis, and infectious diseases, Lichen planus, and Impetigo herpetiformis.

According to some embodiments, the psoriasis is selected from plaque psoriasis, moderate to severe psoriasis, hypertrophic palmoplantar psoriasis, pustular psoriasis and pustular psoriasis.

According to some embodiments, the arthritis is selected from rheumatoid arthritis, psoriatic arthritis, and enthesitis-related arthritis.

According to some embodiments, the infectious disease is a viral, bacterial, or fungal disease.

According to some embodiments, the viral disease is caused by influenza virus infection or SARS-COV-2 infection (COVID-19).

According to some embodiments the disease or disorder is psoriasis or arthritis.

According to some specific embodiments, the disease or disorder is selected from moderate to severe psoriasis, hypertrophic palmoplantar psoriasis, generalized pustular psoriasis, Psoriatic arthritis, Enthesitis-Related Arthritis, active non-radiographic axial spondylarthrites with objective clinical signs of inflammation, ankylosing spondylitis, rheumatoid arthritis, systemic lupus erythematosus (SLE), familial mediterranean fever, and Tumor necrosis factor receptor-associated periodic syndrome (TRAPS).

According to some embodiments psoriasis is plaque psoriasis. According to more specific embodiments the pharmaceutical composition is for use in prevention or treatment of mild to moderate plaque psoriasis.

Still a further aspect of the invention relates to a method for the prevention or treatment of a disorder caused by and/or associated with an overactivity of IL-17, particularly an overactivity of IL-17A and/or IL-17F, comprising administering an effective dose of the VHH antibody as described above or the set of at least two different VHH antibodies as described above or a pharmaceutical composition described above, to a subject in need thereof.

According to some embodiments, the subject is a human subject suffering from a disorder caused by and/or associated with IL-17.

According to some embodiments, the disorder is an inflammatory or an immune disorder.

According to some embodiments, the pharmaceutical composition is for use in the prevention or treatment of asthma, psoriasis, arthritis, hidradenitis suppurativa, inflammatory bowel disease (Crohn's disease, ulcerative colitis), multiple sclerosis, skin cancer, ankylosing spondylitis, uveitis, atopic dermatitis, graft versus host disease, Alzheimer's disease, fatty liver disease, sepsis, ischemic stroke, Parkinson's disease, active non-radiographic axial spondylarthrites with objective clinical signs of inflammation, systemic lupus erythematosus (SLE), familial mediterranean fever (FMF), Tumor necrosis factor receptor-associated periodic syndrome (TRAPS), Hidradenitis Suppurative (HS), Pemphigus vulgaris (PV), Pityriasis rubra pilaris (PRP), alopecia areata, systemic sclerosis, and infectious disease, Lichen planus, and Impetigo herpetiformis.

According to some embodiments, the psoriasis is selected from plaque psoriasis, moderate to severe psoriasis, hypertrophic palmoplantar psoriasis, and pustular psoriasis.

According to some embodiments, the arthritis is selected from rheumatoid arthritis, psoriatic arthritis, and enthesitis-related arthritis.

According to some embodiments, the infectious disease is a viral, bacterial, or fungal disease.

According to some embodiments, the viral disease is caused by influenza virus infection or SARS-COV-2 infection (COVID-19).

According to some specific embodiments, the disease or disorder is selected from moderate to severe plaque psoriasis, hypertrophic palmoplantar psoriasis, generalized pustular psoriasis, psoriatic arthritis, enthesitis-related arthritis, active non-radiographic axial spondylarthrites with objective clinical signs of inflammation, ankylosing spondylitis, rheumatoid arthritis, systemic lupus erythematosus (SLE), familial mediterranean fever, and tumor necrosis factor receptor-associated periodic syndrome (TRAPS).

According to some embodiments the disease or disorder is psoriasis or arthritis.

According to some embodiments the disorder is psoriasis. According to more specific embodiments, the psoriasis is plaque psoriasis. According to yet more specific embodiments the disorder is mild to moderate plaque psoriasis.

According to some embodiments, the composition is administered locally.

According to some embodiments, the composition is administered topically.

According to some embodiments, the administration is by injection. According to a specific embodiment, the administration is by intradermal injection.

Also provided, according to another aspect of the invention is a method of delivering a VHH antibody to a cell comprising contacting the cell with at least one VHH antibody, or a set of VHH antibodies described above.

According to some embodiments, the cell is of a human subject suffering from disorder caused by and/or associated with an overactivity of IL-17, particularly associated with an overactivity of IL-17A and/or IL-17F.

The present invention is explained in more detail by the following Figures and Examples.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1: Sequences of selected anti-IL-17 VHH antibodies.

The figure shows an alignment of sequences from selected VHH antibodies. Residues that deviate from the consensus are highlighted by a gray background. The three CDR regions are indicated.

FIG. 2: Affinities of Re42H11 class VHHs for IL-17 isoforms as measured by Bio-layer interferometry (BLI).

Graphs show binding of indicated IL-17 species to biotinylated VHH antibodies of the Re42H11 class (Class A) that had been immobilized to High Precision Streptavidin biosensors. Binding and dissociation were recorded as wavelength shifts (in nm) on an Octet RED96e instrument (ForteBio/Sartorius). Baselines were recorded by measuring a ‘minus VHH control’ in parallel. On-rates, off-rates, and apparent dissociation constants (KDS) were calculated by the Octet Data Analysis HT 12.0 software), using a mass transport model to fit the data. Note that the immobilization of the VHH to the sensor chips allows for avidity effects, i.e., neighboring VHH molecules may simultaneously bind the same IL-17 dimer, resulting in lower than the actual off-rates. This simulates the binding of a bivalent IgG to an IL-17 dimer. Also note that FIGS. 4-8 and FIG. 11 measured monovalent affinities by using immobilized IL-17 and monovalent VHH antibodies as an analyte. All VHH antibodies characterized in BLI experiments were produced by periplasmic expression in E. coli.

FIG. 3: Affinities of Bm17B02 class VHH antibodies for IL-17 isoforms.

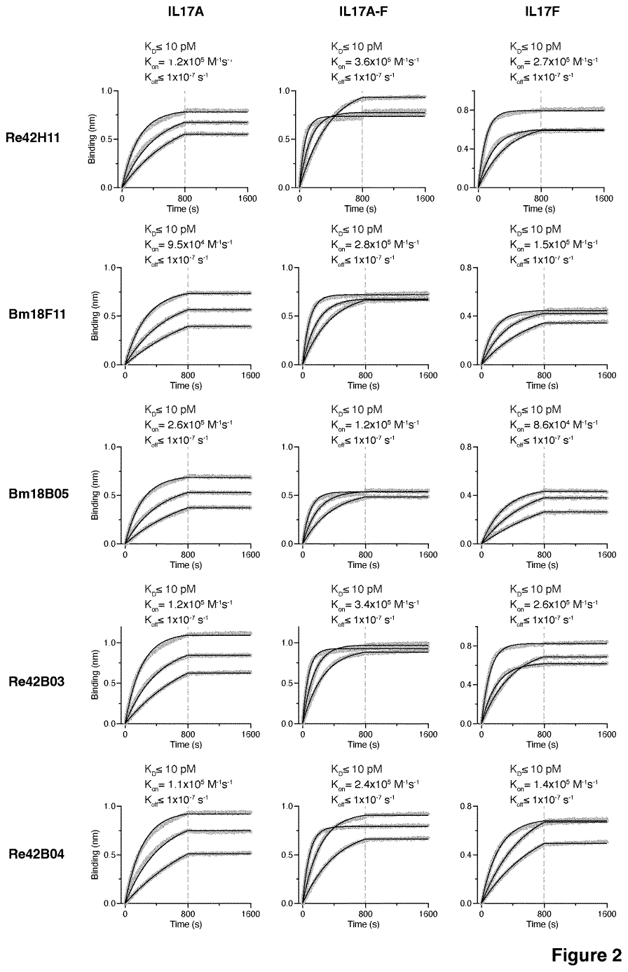

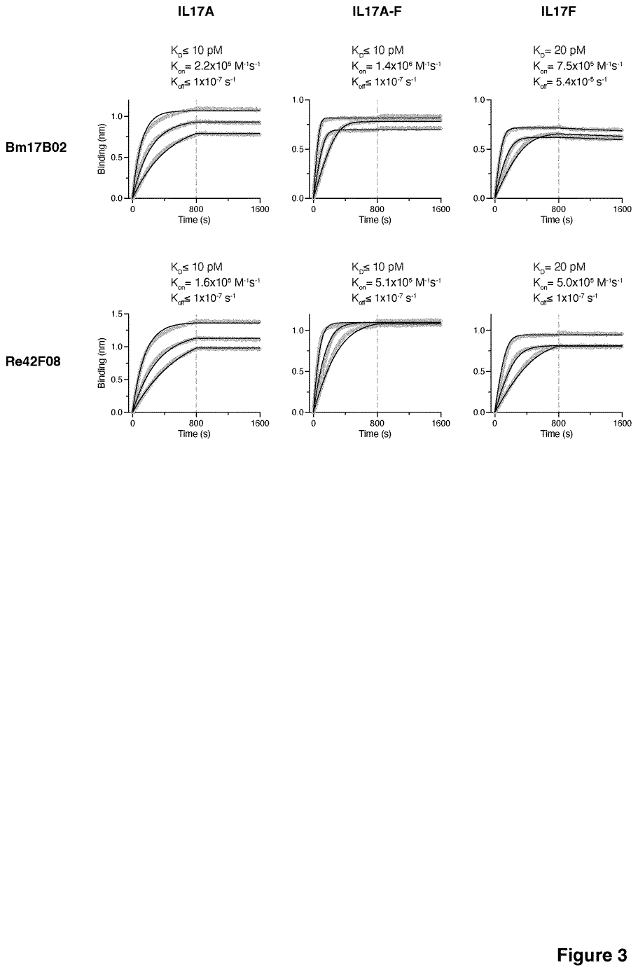

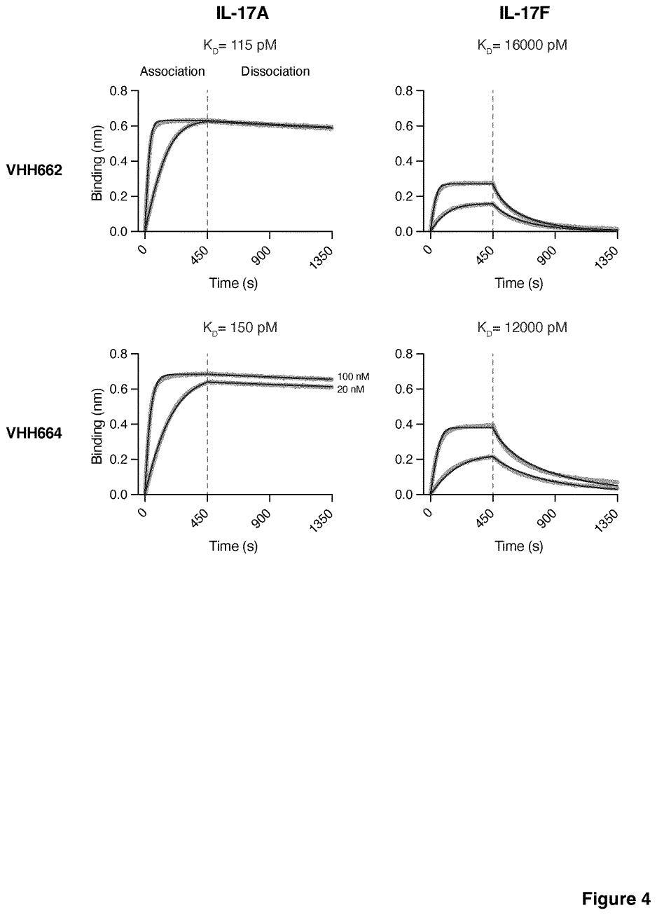

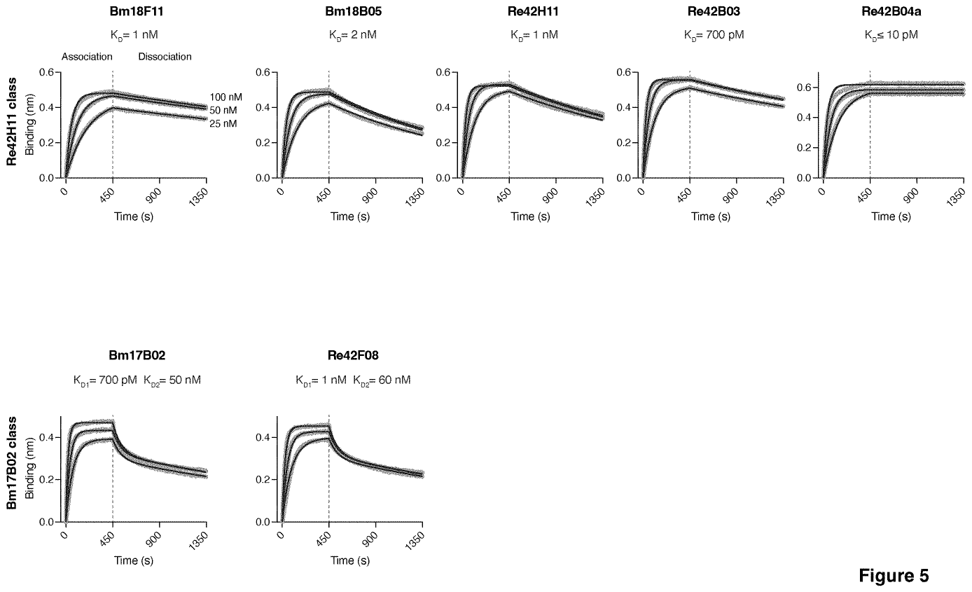

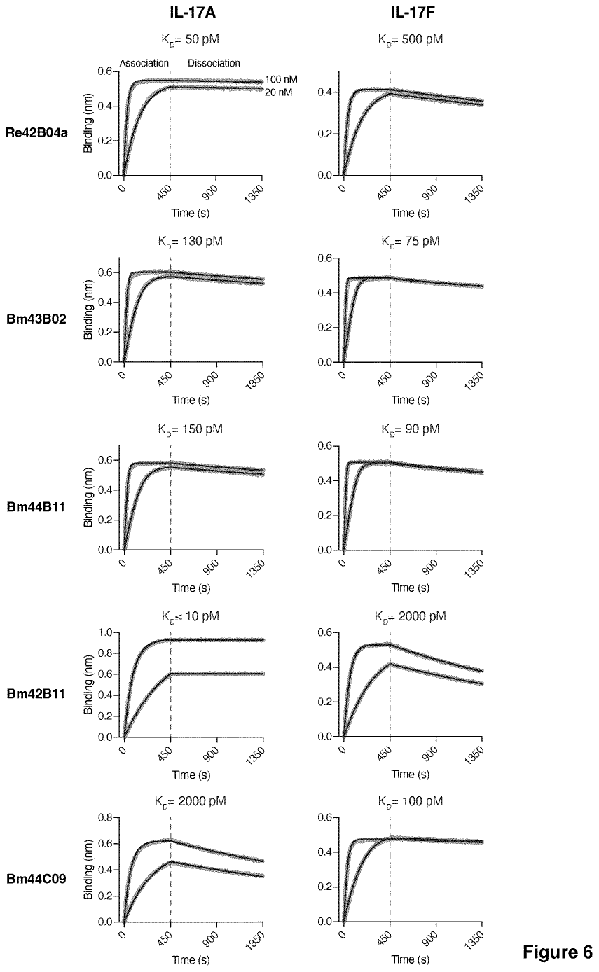

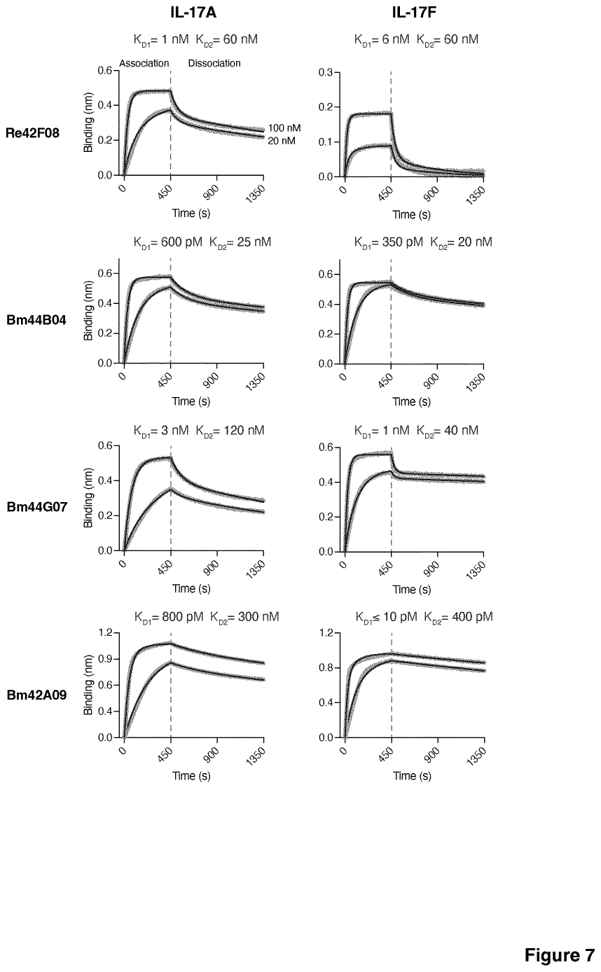

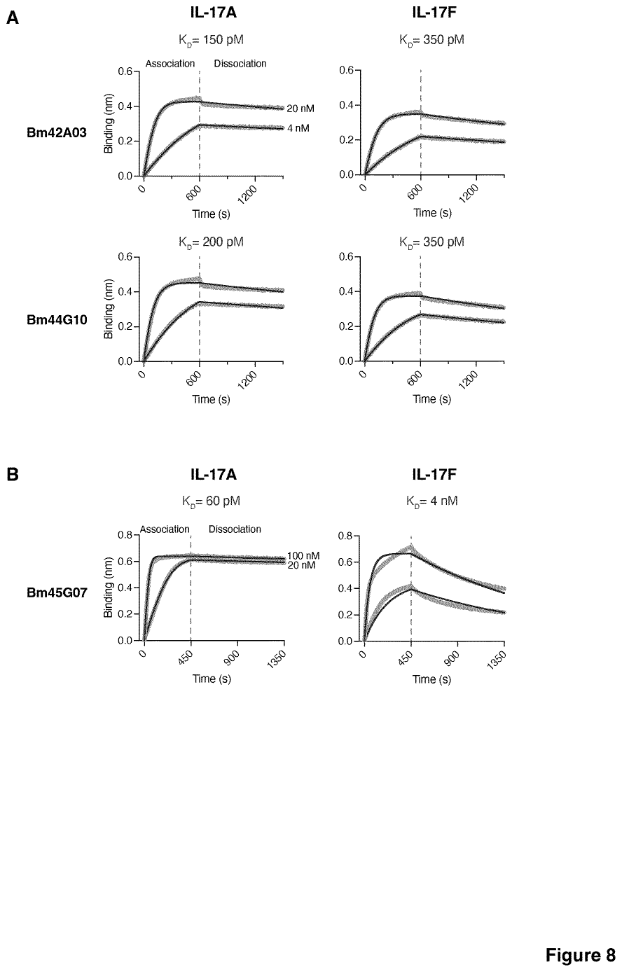

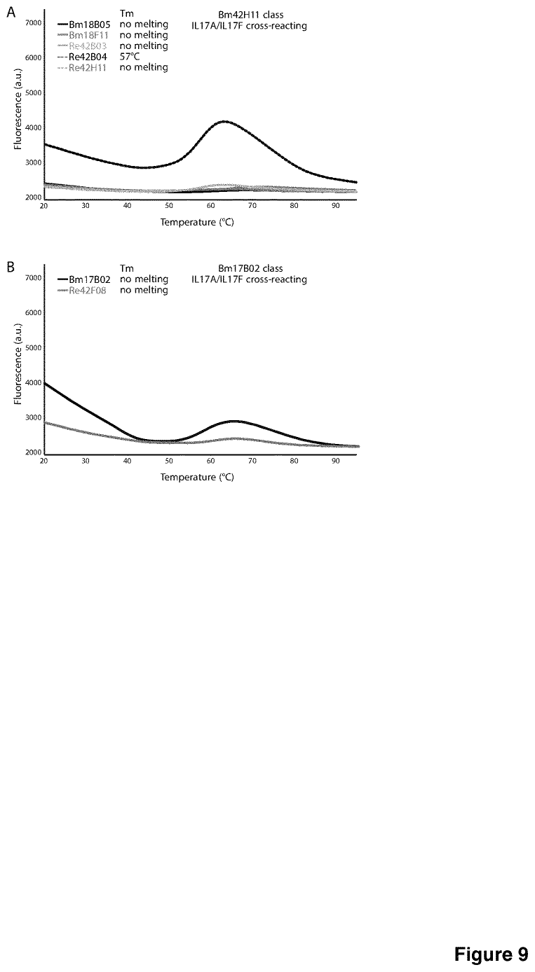

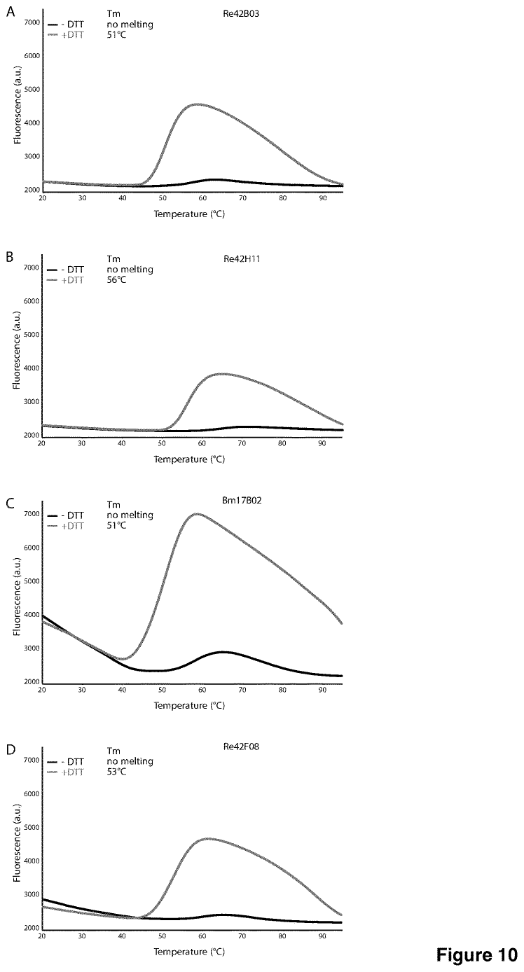

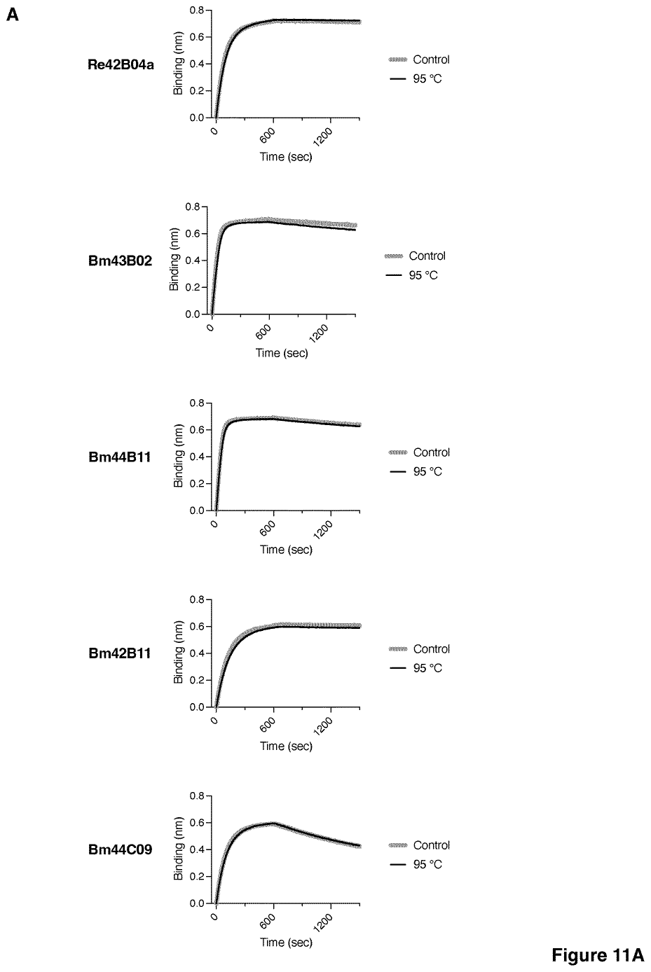

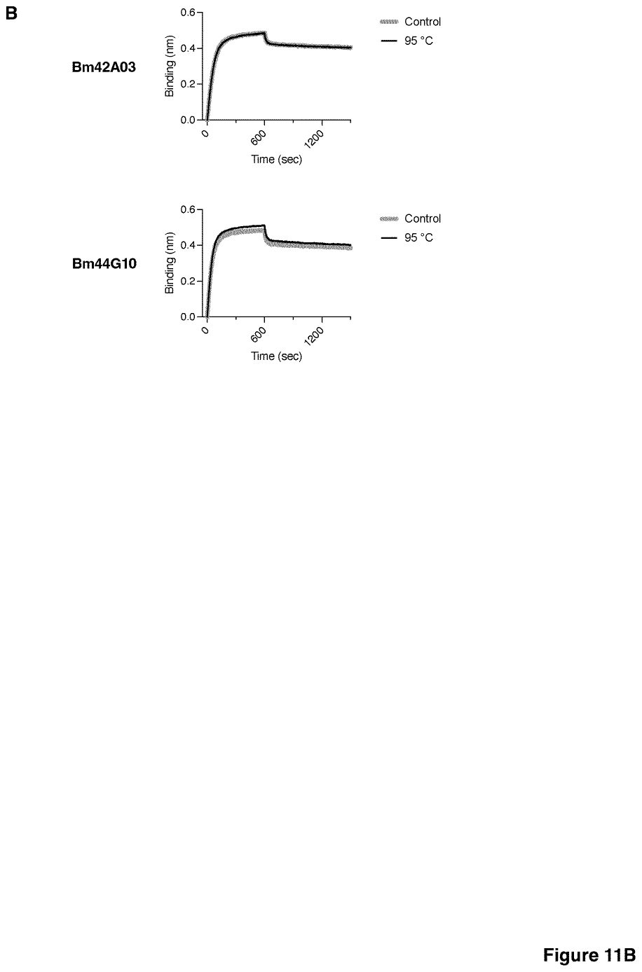

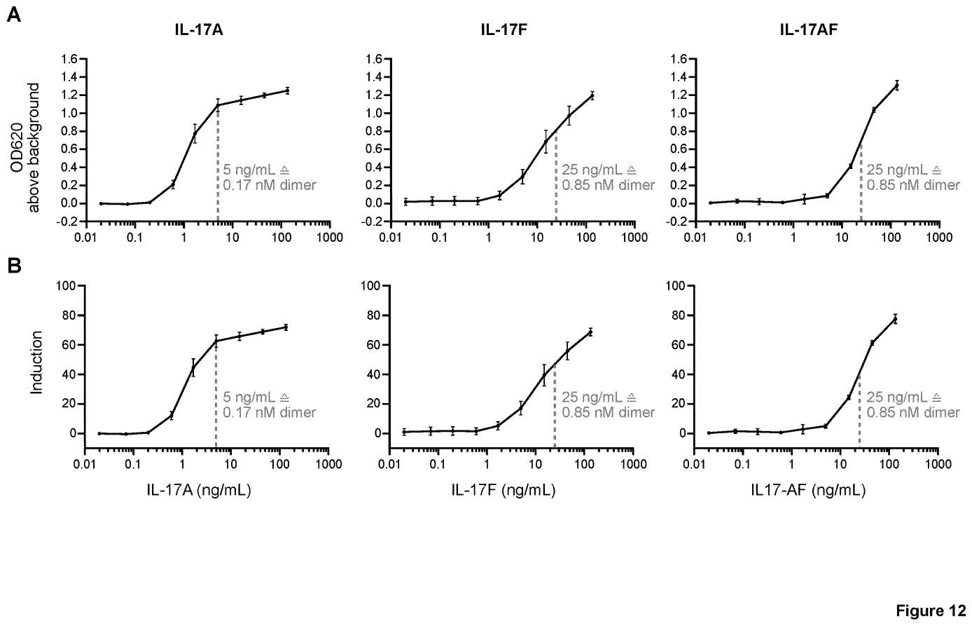

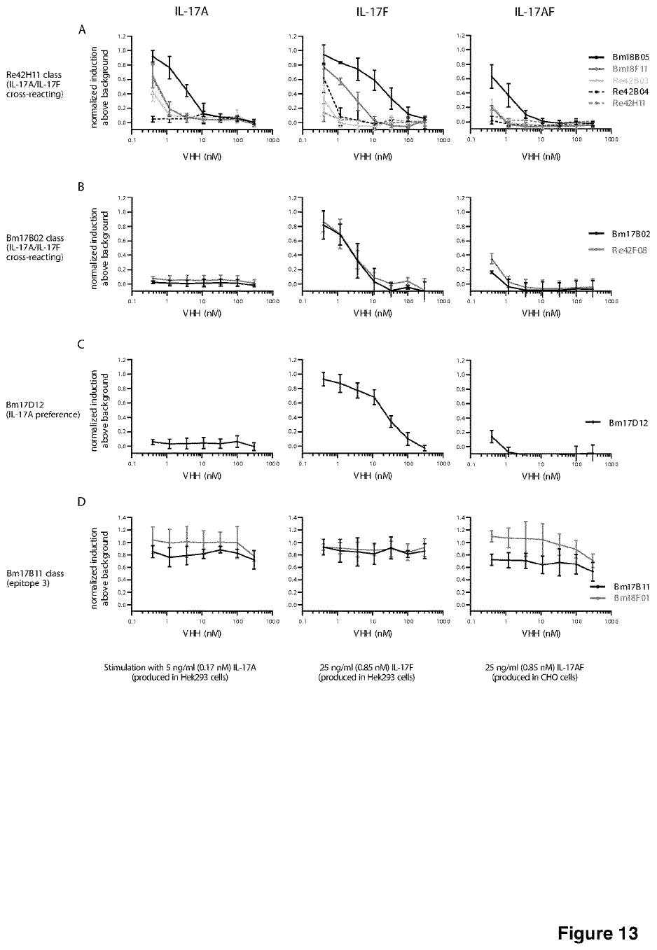

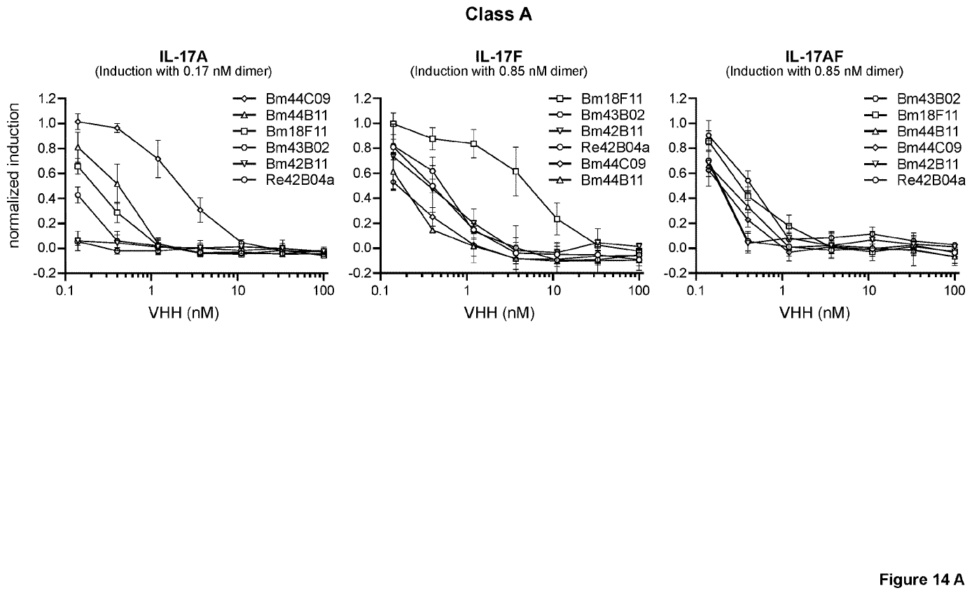

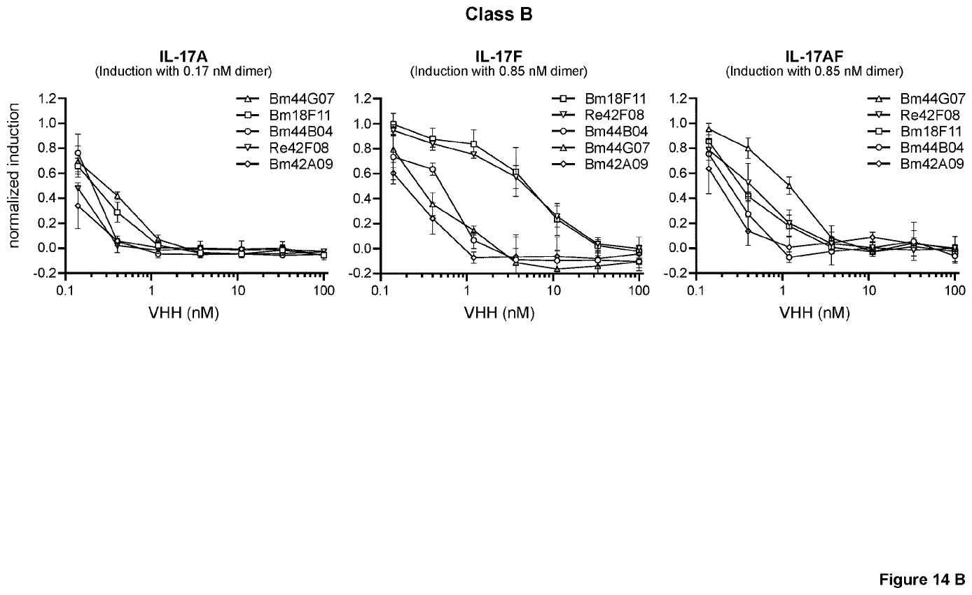

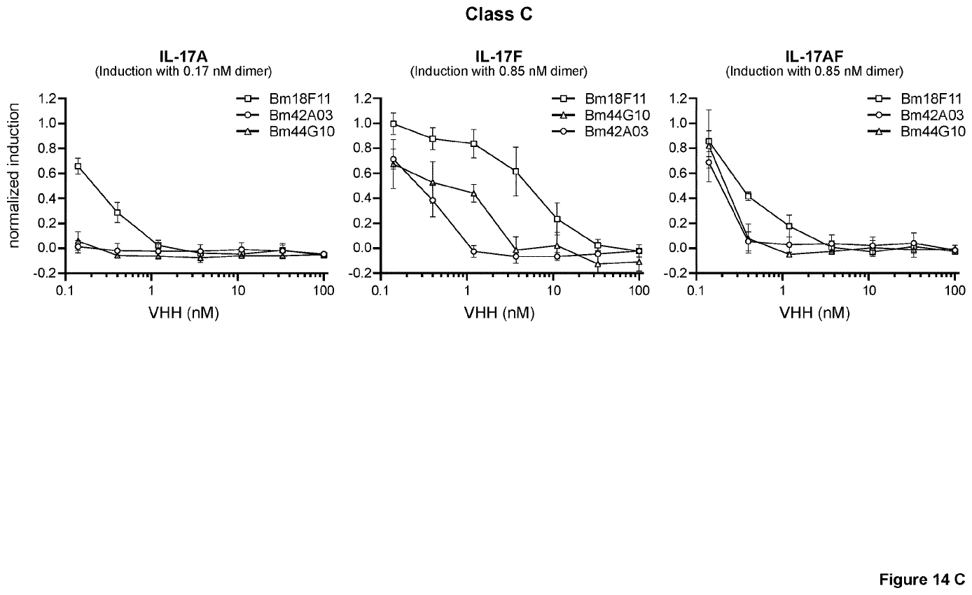

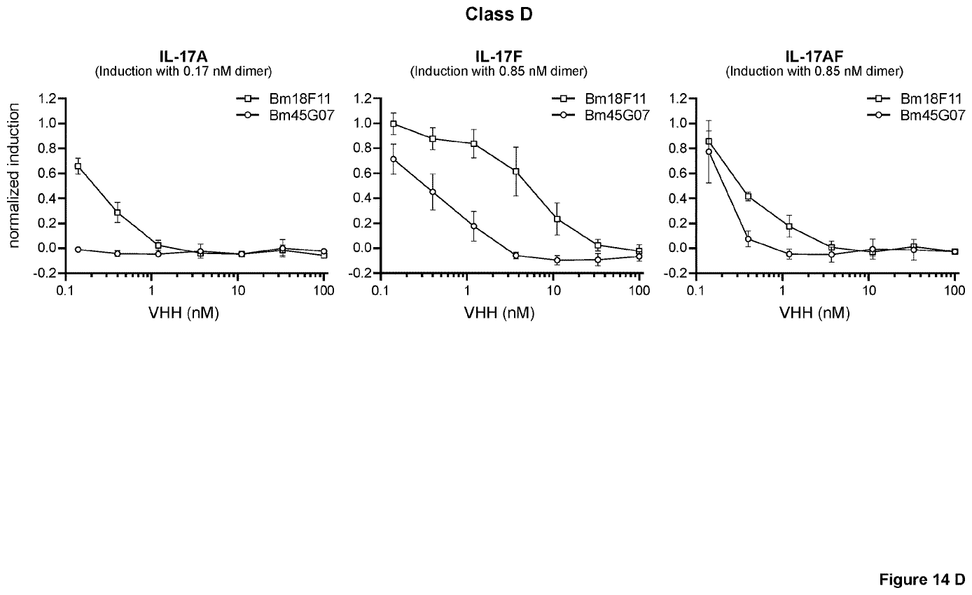

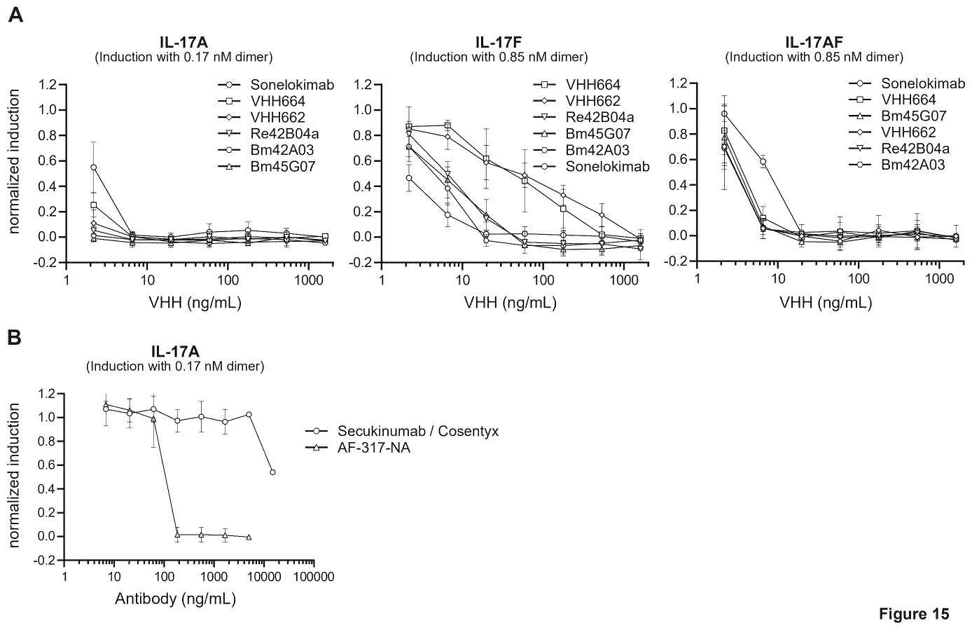

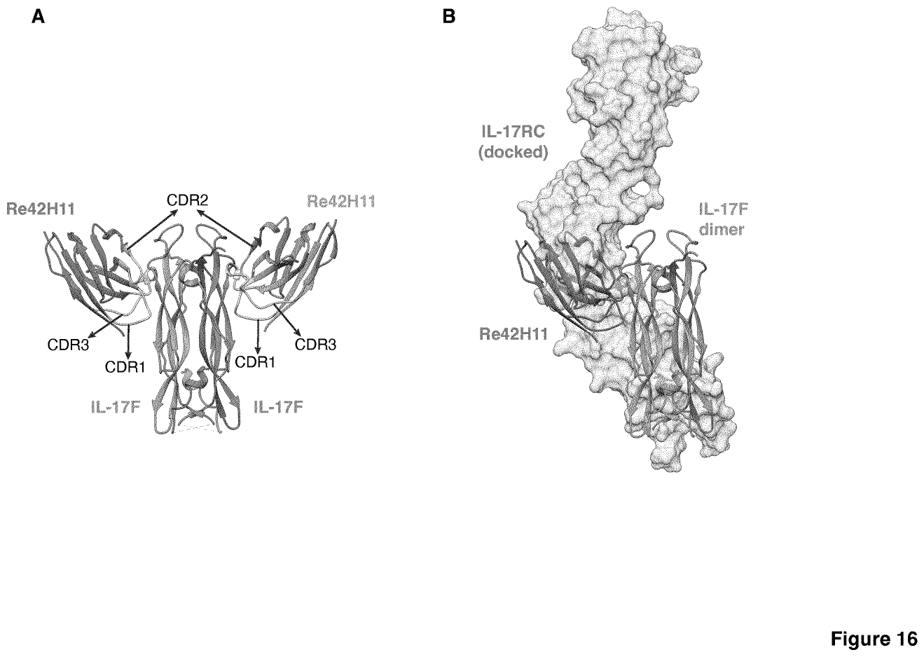

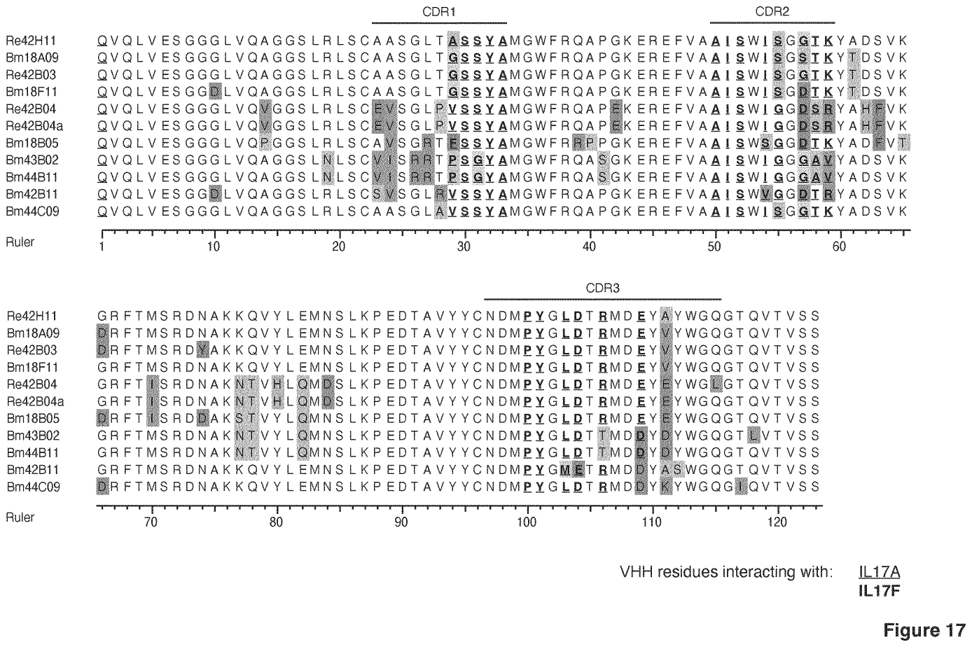

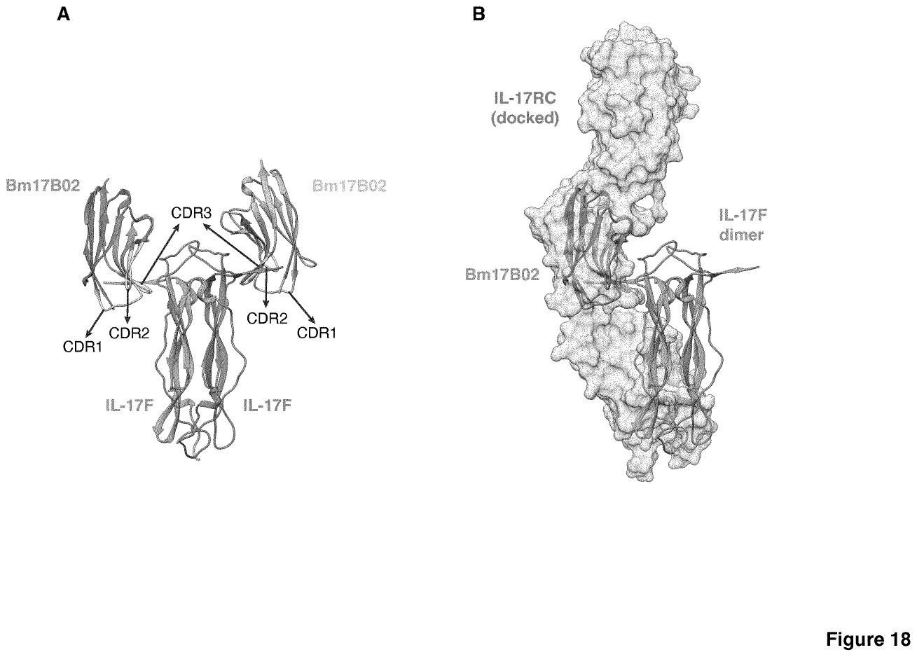

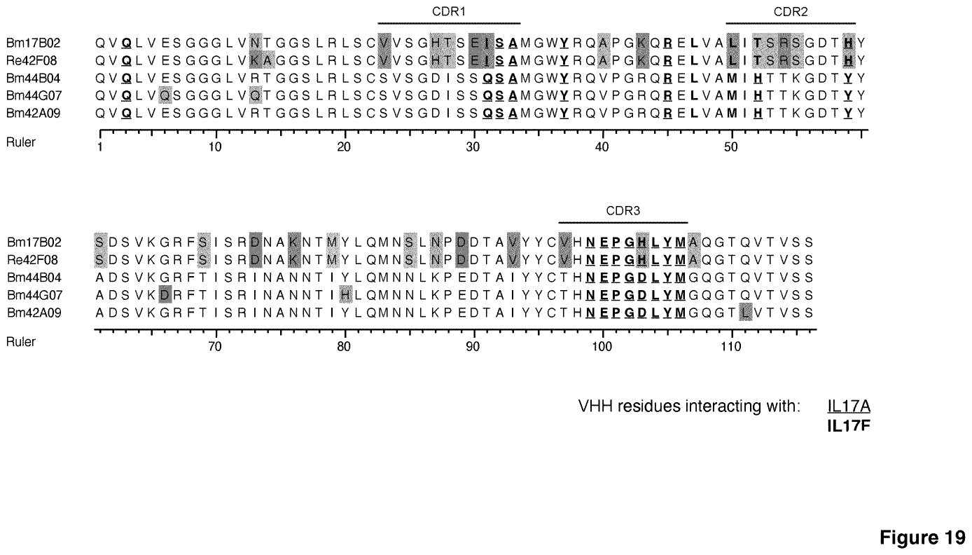

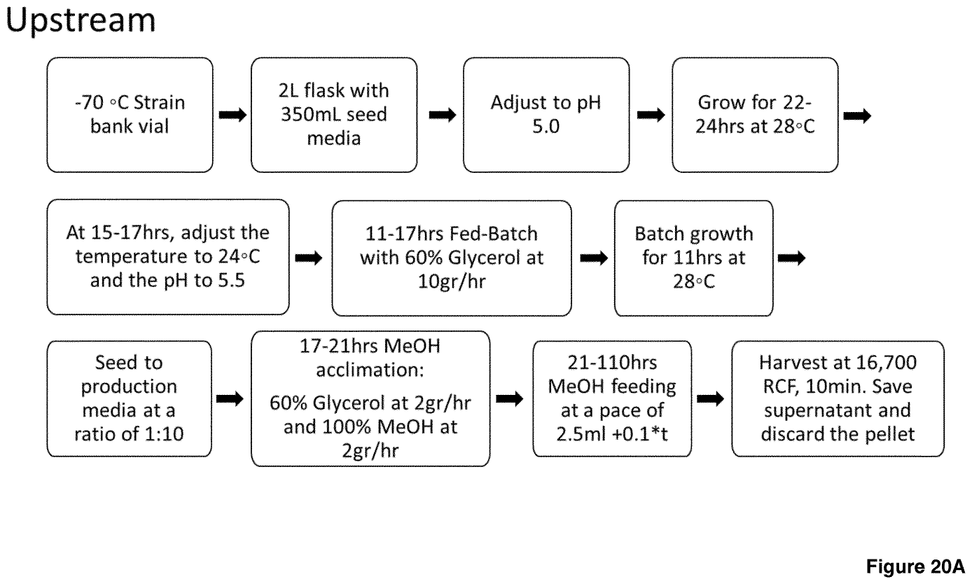

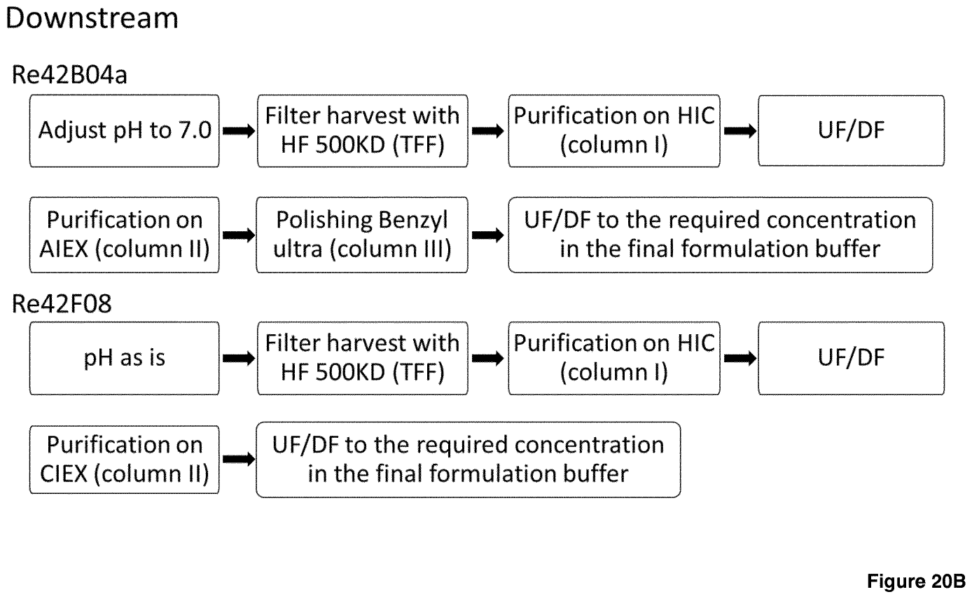

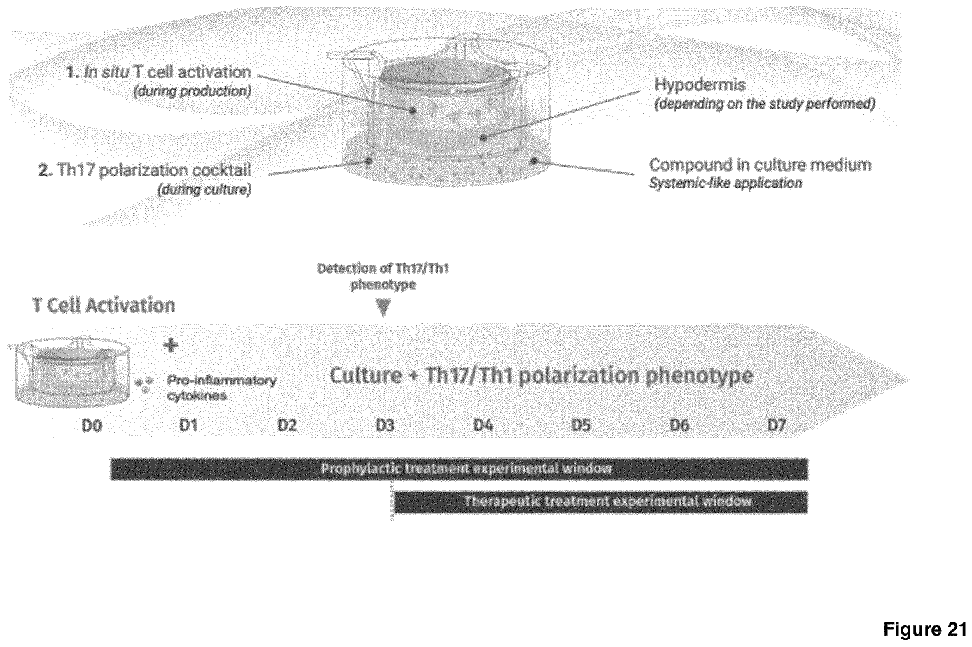

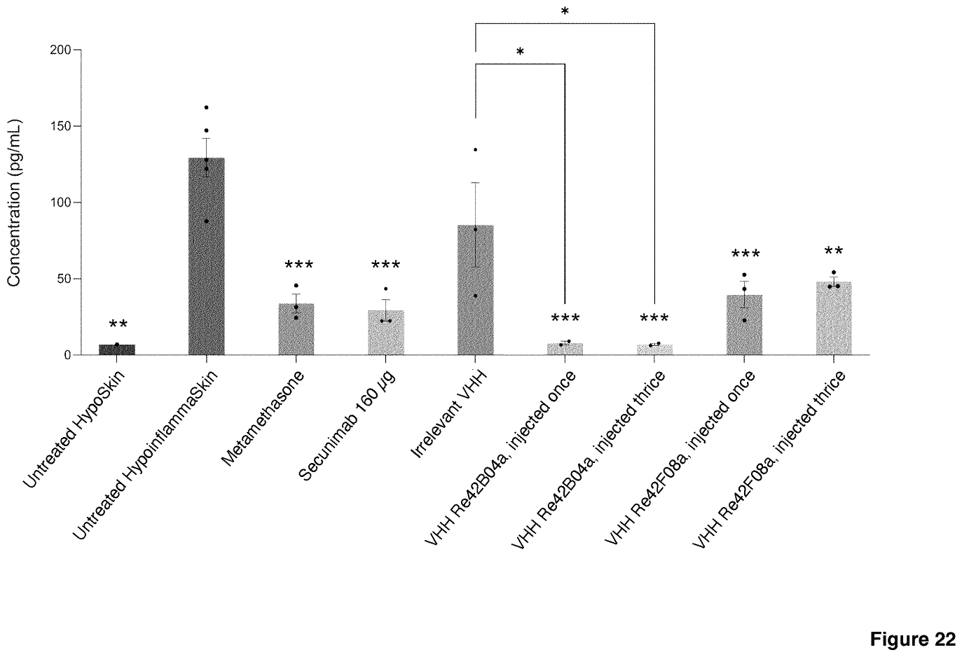

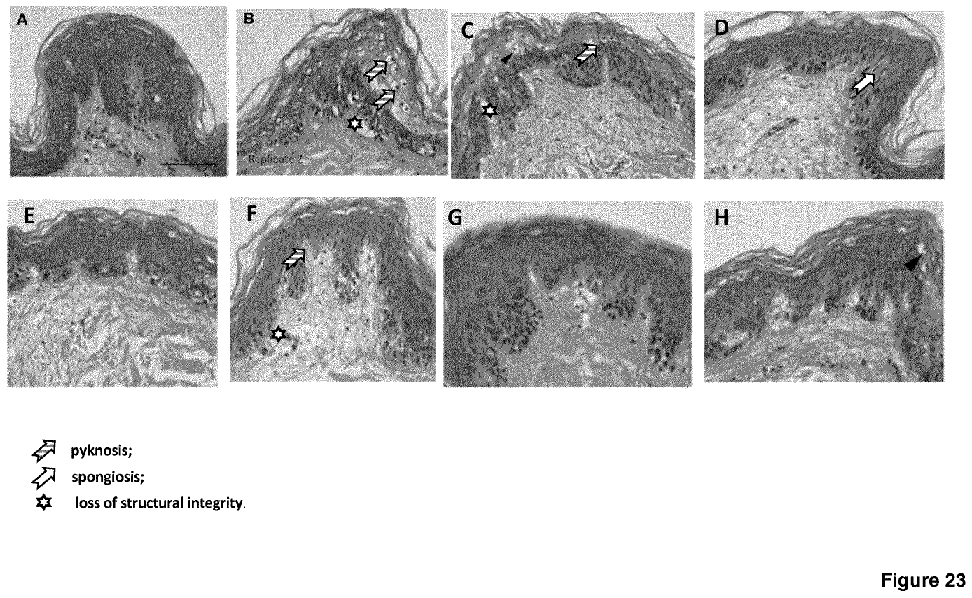

BLI measurements as in FIG. 2 but for Bm17B02 and Re42F08 VHH antibodies of Class B.

FIG. 4: Affinities of the control VHHs VHH662 and VHH664 for IL-17 isoforms.

Human IL-17A and IL-17F were produced with a C-terminal Avi-tag and enzymatically biotinylated by recombinant BirA (Beckett et al., 1999) and immobilized at a concentration of 100 nM for 200 seconds to High Precision Streptavidin biosensors on an Octet RED96e instrument (Sartorius), using Phosphate-Buffered Saline (PBS) pH 7.4, 0.02% (w/v) Tween 20 and 0.1% (w/v) bovine serum albumin (BSA) as an assay buffer. 20 and 100 nM of VHH662 or VHH664 antibodies were then allowed to bind for 450 seconds and, subsequently, to dissociate for 900 seconds. Binding and dissociation were recorded as wavelength shifts (in nm). Baselines were recorded by measuring a ‘minus VHH control’ in parallel. Curves (gray) were fitted with a mass transport model. (for Re42H11 class VHHs, Class A) and 2:1 heterogenous model (for Bm17B02 class VHHs, Class B). Fitted curves and calculated dissociation constants (K_{D}s) are shown in black. Note that this setup measures affinities independently of avidity effects because the VHH antibodies are supplied as the analyte and are thus monomeric prior to target binding.

FIG. 5: Affinities of first generation Re42H11 and Bm17B02 class VHHs for IL-17A.

BLI experiments were performed as described in FIG. 4 with first generation VHH antibodies as analytes (using 25, 50 and 100 nM VHH) and biotinylated IL-17A immobilized to sensor chips. For dissociation constant (K_{D}ps) calculations, curves (gray) were fitted with a mass transport model (for Re42H11 class VHHs, Class A) or 2:1 heterogenous model (for Bm17B02 class VHHs, Class B). The lower affinity component in class B VHHs is probably due to excess VHH molecules binding to a secondary site on IL-17.

FIG. 6: Affinities of second generation Re42H11 class VHHs for IL-17 isoforms.

BLI experiments were performed as described in FIG. 4, analyzing binding of indicated Re42H11 class VHHs as analytes to biotinylated IL-17A or IL-17F.

FIG. 7: Affinities of second generation Bm17B02 class VHHs for IL-17 isoforms.

Indicated VHHs (class B) were analyzed by BLI as described in FIG. 4.

FIG. 8: Affinities of the other VHHs (class C and D) for IL-17 isoforms.

VHHs Bm42A03, Bm44G10 (class C) and Bm45G07 (class D) were analyzed by BLI as described in FIG. 4.

FIG. 9: Thermal stability of disclosed VHH antibodies.

VHH antibodies were subjected to Differential scanning fluorimetry (DSF) as detailed in Example 4. Thermal unfolding is here measured as an enhanced fluorescence of added SYBR Orange following a stepwise increase in temperature with 532 nm excitation and a 555 nm long pass filter. Melting temperatures are defined as the inflection point of the first melting peak. Re42B04 is the only VHH of this series showing melting peak; this peak is, however, rather low, indicating only a local and not a global melting of the VHH.

FIG. 10: Thermal stabilities under non-reducing and disulfide bond-reducing conditions.

Thermal stability assays were performed as in FIG. 9. Here, however, controls in the presence of 10 mM dithiothreitol (DTT) are shown. DTT reduces the structural, stabilizing disulfide bond.

FIG. 11: Thermostability of VHHs determined by BLI.

Indicated VHH antibodies (1 μM) of Class A (FIG. 11A) and C (FIG. 11B) were incubated at room temperature or at 95° C. for 10 min and centrifuged for 20 min at 20,000 g. The supernatants were diluted 20-fold (to 50 nM) and analyzed for IL-17A-binding by BLI.

FIG. 12: Dose-response of HEK-Blue™ IL-17 reporter cells to recombinant IL-17 cytokines.

Cells of the HEK-Blue™ IL-17 reporter line were stimulated with increasing concentrations of human IL-17A, IL-17F, and IL-17AF. Stimulation results in the secretion of an embryonic alkaline phosphatase (SEAP) reporter, whose activity was determined colorimetrically using QUANTI-Blue™ as a substrate and measuring the resulting absorbance at 620 nm. The OD620 values are plotted in (A). The plots in (B) depict the range of induction as calculated by dividing the OD620 readings at the respective IL-17 dilution by the OD620 value of the minus IL-17 control) The results are the mean±SD of one experiment measured in triplicate. Dashed vertical lines indicate the IL-17 concentrations that were used in the subsequent VHH neutralization experiments (FIGS. 13-14).

FIG. 13: Neutralization of IL-17A and IL-17F by first generation VHH antibodies.

Plots show the expression of a phosphatase reporter by HEK-Blue™ IL-17 cells, following induced by 0.17 nM IL-17A homodimers, 0.85 nM IL-17F homodimers, or 0.85 nM IL-17AF heterodimers pre-incubated with indicated concentrations of anti-IL-17 VHH antibodies. A number of 1.0 indicates (full) induction in the absence of a VHH, and a number of 0 indicates no induction above the background of the minus VHH controls. Graphs show neutralization by Re42H11 and Bm17B02 class members (Class A and B respectively), each measured in triplicates.

FIG. 14: Neutralization of IL-17A, F and AF by VHH antibodies of classes A-D.

Plots show the normalized expression of a phosphatase reporter induced by 0.17 nM IL-17A homodimers, 0.85 nM IL-17F homodimers, or 0.85 nM IL-17AF heterodimers pre-incubated with indicated concentrations of anti-IL-17 VHH antibodies. A number of 1.0 indicates (full) induction in the absence of a VHH, and a number of 0 indicates no induction above the background of the untreated cells. Graphs show neutralization by (A) class A (Re42B04a), (B) class B (Re42F08), (C) class C (Bm42A03), and (D) class D (Bm45G07). Bm18F11 is included for reference in all graphs. The results are the mean±SD of three independent experiments, each measured in triplicate.

FIG. 15: Neutralization of IL-17A, IL-17F and IL-17AF by control VHH antibodies.

Plots show the normalized expression of a phosphatase reporter induced by 0.17 nM IL-17A homodimers, 0.85 nM IL-17F homodimers, or 0.85 nM IL-17AF heterodimers each pre-incubated with indicated concentrations of anti-IL-17 VHH antibodies. A number of 1.0 indicates (full) induction in the absence of a VHH, and a number of 0 indicates no induction above the background of the untreated cells. (A) The graph shows the neutralization of representative members of class A (Re42B04a), class C (Bm42A03), and class D (Bm45G07) in comparison to the control VHH antibodies VHH662, VHH664 and a Sonelokimab Biosimilar (Proteogenix PX-TA1606). Sonelokimab is a tandem fusion of three VHHs directed against IL-17A, IL-17F, and albumin. Because of its higher molecular weight and the presence of two IL-17 binding sites in Sonelokimab, identical mass concentrations (ng/mL) were compared. The results are the mean±SD of three independent experiments, each measured in triplicate. (B) Neutralization of IL-17A by a Secukinumab/Cosentyx® Biosimilar (Proteogenix PX-TA1234) and a research-grade anti IL-17A antibody (R&D Systems AF-317-NA) as control. The results are the mean±SD of two independent experiments, each measured in triplicate.

FIGS. 16A-B: Crystal structure of the VHH class A member Re42H11 bound to an IL-17F dimer.

His14-ScSUMO-tagged Re42H11 was co-expressed with His14-BdNEDD8-tagged human IL-17F (residues 39-163) in E. coli SHuffle Express (New England Biolabs). The complex was purified by Ni-chelate chromatography, by immobilizing the complex constituents via the His14 tag, followed by two successive tag-cleaving elutions (Frey and Görlich, 2014) with the bdNEDP1 protease (cleaving NEDD8) and Sculp1 protease (cleaving SUMO). The complex Re42H11.IL-17F was recovered in the second elution step and further purified by size exclusion chromatography. The complex was crystallized, an x-ray diffraction dataset was recorded at the Swiss Light Source synchrotron, and the structure was solved by molecular replacement to a resolution of 2.8 Å and an Rfree of 0.28.

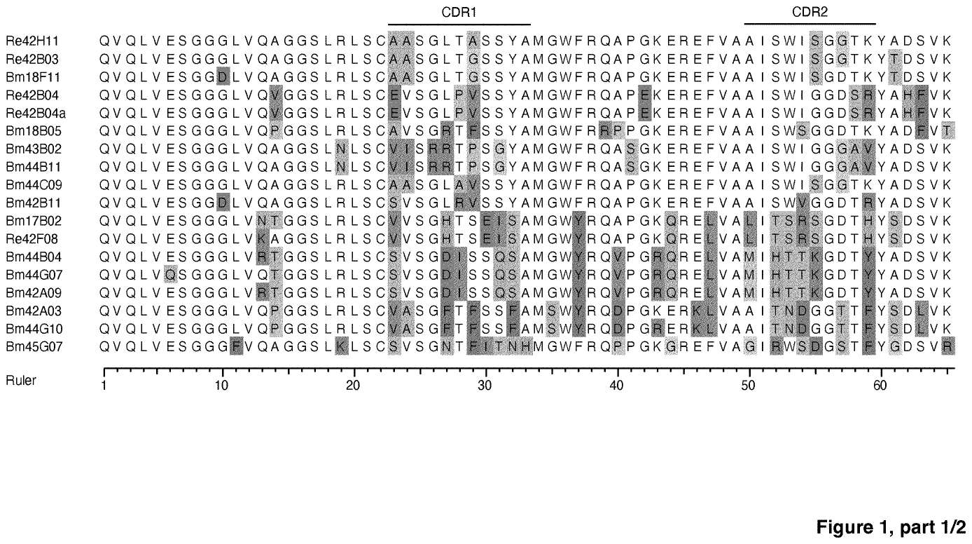

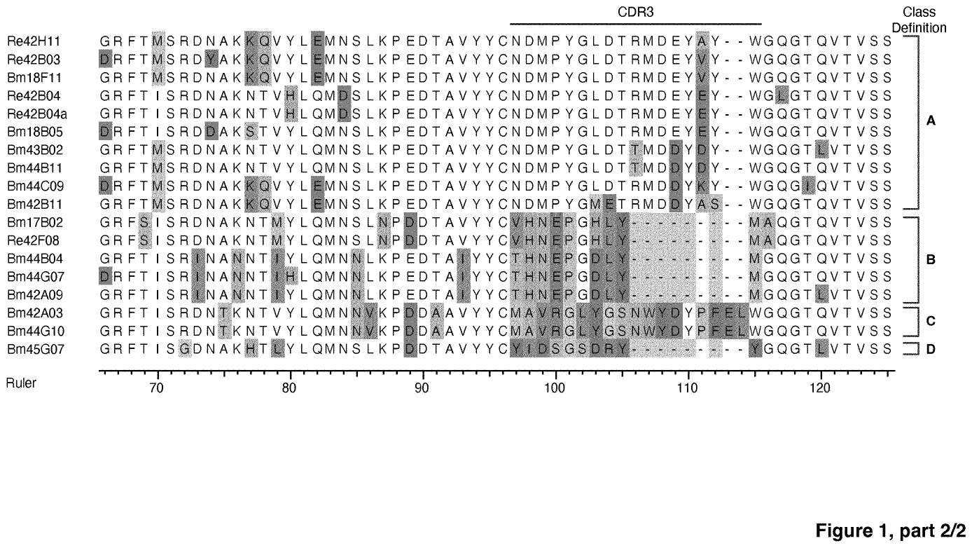

FIG. 16A. Crystal structure of the tetrameric Re42H11.IL-17F complex in ribbon representation. CDR regions (defined in FIG. 1) are highlighted in yellow and indicated accordingly.

FIG. 16B. IL-17 receptor IL17-RC (semi-transparent gray surface) was docked onto the IL-17F dimer, based on the alignment of IL-17F with the IL-17RC ECD.IL-17F complex (PDB ID 6HG4, Goepfert et al., 2020).

FIG. 17: Sequence alignment of class A anti-IL-17 VHHs with indicated IL-17-interacting residues.

IL-17 interacting residues of Class A VHH antibodies were identified from the crystal structure (FIG. 16) or by homology-modelling based on this structure. IL-17A interacting residues are underlined, IL-17F interacting residues are printed in bold.

FIG. 18: Crystal structure of the class B VHH Bm17B02 bound to an IL-17F dimer.

Complex formation and structure determination were as described in FIG. 16 with the only difference being a His14-ScSUMO-tagged Bm17B02 used for co-expression and complex formation.

FIG. 18A. Crystal structure of the tetrameric Bm17B02.IL-17F complex in ribbon representation. CDR regions (shown in FIG. 1) are highlighted in yellow and indicated accordingly.

FIG. 18B. IL17-RC (semi-transparent gray surface) was docked onto the IL-17F dimer as described in FIG. 16.

FIG. 19: Sequence alignment of class B VHHs, highlighting IL-17-interacting residues.

IL-17 interacting residues of Class B VHH antibodies were identified from the crystal structure (FIG. 18) or by homology-modelling based on this structure. IL-17A interacting residues are underlined, IL-17F interacting residues are printed in bold.

FIGS. 20A and 20B: Manufacturing of VHH antibodies in Pichia pastoris.

The schemes describe the manufacturing of the VHH antibodies Re42B04a and Re42F08 in Pichia pastoris. FIG. 20A upstream process. FIG. 20B downstream process. Abbreviations used: MeOH—Methanol, t—time, RCF—Relative centrifugal force, HF—Hollow Fiber, TFF—Tangential Flow Filtration, HIC—Hydrophobic interaction chromatography, AIEX—Anion exchange, CIEX—cation exchange, UF/DF—Ultrafiltration/Diafiltration.

FIG. 21: Full-thickness human ex vivo skin model.

Schematic presentation of the human ex vivo skin model “Inflammaskin” reproducing key features and inflammatory responses observed in psoriatic lesions.

FIG. 22: Evaluation of IL-17A release in a psoriatic ex vivo model.

The model is comprised of healthy human skin samples that are treated with a Th polarization cocktail to induce the psoriatic phenotype in Hypo InfammaSkin samples. Details of the study design are shown in Table 4. VHH antibodies Re42B04a and Re42F08 were injected once or thrice intradermally into the skin samples. Positive control samples included betamethasone and Secukinumab that are used for Psoriasis treatment, Betamethasone is an anti-inflammatory steroid whereas Secukinumab is a monoclonal antibody that targets IL-17A. The irrelevant VHH antibody Re32D03 (SEQ ID NO. 64, directed against the spike protein of SARS-COV2) was used as a negative control. HypoSkin and HypoInflammaSkin samples were cultured for 7 days. On day 7 of culture, supernatants were sampled and IL-17A concentrations were measured using an MSD kit (K15076K, MesoScale Discovery). Concentrations are expressed in pg/mL, with values for each replicate (dots) plotted, and the average and standard error of mean (SEM) shown for each condition. The mean was calculated from the detectable/QC controlled values. The graph shows all the replicates for each condition. A statistical analysis (by One-Way ANOVA) was performed, between each condition and the Untreated HypoInflammaSkin control. Additionally, a One-Way ANOVA test was performed to evaluate the differences between each condition and the irrelevant VHH Re32D03; * p<0.05; ** p<0.01; *** p<0.001; **** p<0.0001.

FIGS. 23A-H: Evaluation of human ex vivo psoriatic skin samples following treatment with anti-IL17 VHHs

Skin samples from the study described in Table 4 and analyzed in FIG. 22 were collected at day 7, fixed, stained with H&E and analyzed by microscopy. FIG. 23A: HypoSkin sample (not treated with pro-inflammatory cytokines). Images B-H show InflammoSkin samples, treated with the Th polarization cocktail. FIG. 23B: untreated psoriasis-induced tissue (negative control), FIG. 23C: treated with Betamethasone (positive control), FIG. 23D: treated with Secukinumab (positive control), FIG. 23E: VHH antibody Re42B04a injected once, FIG. 23F: VHH antibody Re42B04a injected thrice, FIG. 23G: VHH antibody Re42F08 injected once, and FIG. 23H: VHH antibody Re42F08 injected thrice.

### DETAILED DESCRIPTION OF THE INVENTION

The present invention relates to VHH antibodies recognizing human IL-17 polypeptides, including IL-17A, IL-17F and IL-17AF.

It is challenging and not obvious to obtain VHH antibodies that block IL-17 and bind tightly to both, IL17A and IL17F, because the neutralizing epitope has a few amino acid exchanges between 17A and 17F. The present invention provides VHH antibodies (e.g., Bm43B02) with picomolar affinity to both 17A and 17F (measured as binding affinity of a monomeric VHH, to an immobilized human IL-17A or IL-17F homodimer). It is remarkable to notice that when compared to previously published VHH antibodies targeting IL-17, (for example those disclosed in WO2012156219), the VHH antibodies of the present invention have higher affinity to the IL-17 isoforms, in particular to IL-17F.

The binding data for the novel VHH antibodies of the present invention have been confirmed in cell-based assays for blocking IL-17 receptor activation and in ex-vivo human skin models.

### VHH Antibodies

The sequences of the CDRs of VHH antibodies of the present invention, and control antibodies are listed in the following Table 1 while Table 2 lists the sequences of the full-length VHH antibodies.

TABLE 1
Sequences of anti-IL-17 VHH antibodies.

| | Seq | | Seq | | Seq | | Seq |
|---|---|---|---|---|---|---|---|
| | ID | | ID | | ID | | ID |
| VHH | No. | CDR1 | No. | CDR2 | No. | CDR3 | No. |
| Class A | | | | | | | |
| Re42H11 | 5 | AASGLTASSYA | 6 | AISWISGGTK | 7 | NDMPYGLDTRMDEYAYW | 8 |
| Re42B03 | 12 | AASGLTGSSYA | 10 | AISWISGGTK | 7 | NDMPYGLDTRMDEYVYW | 11 |
| Bm18F11 | 13 | AASGLTGSSYA | 10 | AISWISGDTK | 14 | NDMPYGLDTRMDEYVYW | 11 |
| Re42B04 | 15 | EVSGLPVSSYA | 16 | AISWIGGDSR | 17 | NDMPYGLDTRMDEYEYW | 18 |
| Re42B04a | 19 | EVSGLPVSSYA | 16 | AISWIGGDSR | 17 | NDMPYGLDTRMDEYEYW | 18 |
| Bm18B05 | 20 | AVSGRTFSSYA | 21 | AISWSGGDTK | 22 | NDMPYGLDTRMDEYEYW | 18 |
| Bm43B02 | 23 | VISRRTPSGYA | 24 | AISWIGGGAV | 25 | NDMPYGLDTTMDDYDYW | 26 |
| Bm42B11 | 28 | SVSGLRVSSYA | 29 | AISWVGGDTR | 30 | NDMPYGMETRMDDYASW | 31 |
| Bm44C09 | 32 | AASGLAVSSYA | 33 | AISWISGGTK | 7 | NDMPYGLDTRMDDYKYW | 34 |
| Class B | | | | | | | |
| Bm17B02 | 35 | VVSGHTSEISA | 36 | LITSRSGDTH | 37 | VHNEPGHLYM | 38 |
| Bm44B04 | 40 | SVSGDISSQSA | 41 | MIHTTKGDTY | 42 | THNEPGDLYM | 43 |
| Bm44G07 | 44 | SVSGDISSQSA | 41 | MIHTTKGDTY | 42 | THNEPGDLYM | 43 |
| Class C | | | | | | | |
| Bm42A03 | 49 | VASGFTFSSFA | 50 | AITNDGGTTF | 51 | MAVRGLYGSNWYDYPFELW | 52 |
| Bm44G10 | 53 | VASGFTFSSFA | 50 | AITNDGGTTF | 51 | MAVRGLYGSNWYDYPFELW | 52 |
| Class D | 54 | SVSGNTFITNH | 55 | GIRWSDGSTF | 56 | YIDSGSDRYY | 57 |
| Bm45G07 | | | | | | | |
| Controls | | | | | | | |
| Bm17D12 | 65 | AASGFTLDDYV | 68 | CISSISNSDGRAY | 69 | AAERGRIDPDVAGYFLRES | 70 |
| | | | | | | YVMDYW | |
| Bm17B11 | 66 | AASGFIFSHYW | 71 | GINTGGRTTV | 72 | TALRGDYGKDLTSLDEYEY | 73 |
| | | | | | | W | |
| Bm18F01 | 67 | AASGFIFSHYW | 74 | AINTGGRNTV | 75 | TALRGDYGKDLVSLDEFEY | 76 |
| | | | | | | W | |
| VHH662* | 47 | AASGRANSINW | 58 | GIRWSDAYTE | 59 | VLDLSTVRYW | 60 |
| VHH664* | 48 | AASGRTYDA | 61 | AISGSGDDTY | 62 | ATRRGLYYVWDSNDYENW | 63 |

*These VHH antibodies of Ablynx were disclosed in WO2012156219 are provided herein only for comparison as they were compared in some of the experiments, with the VHH antibodies of the present invention.

TABLE 2
Sequences of VHH antibodies

| | | SEQ ID |
|---|---|---|
| Name | Amino acid sequence | NO. |
| Class A | | |
| Re 42B04a | QVQLVESGGGLVQVGGSLRLSCEVSGLPVSSYAMGWFRQAPEKEREFVAAISWIGGDSRYAH | 19 |
| | FVKGRFTISRDNAKNTVHLQMDSLKPEDTAVYYCNDMPYGLDTRMDEYEYWGQGTQVTVSS | |
| Re42B04 | QVQLVESGGGLVQVGGSLRLSCEVSGLPVSSYAMGWFRQAPEKEREFVAAISWIGGDSRYAH | 15 |
| | FVKGRFTISRDNAKNTVHLQMDSLKPEDTAVYYCNDMPYGLDTRMDEYEYWGLGTQVTVSS | |
| Re42H11 | QVQLVESGGGLVQAGGSLRLSCAASGLTASSYAMGWFRQAPGKEREFVAAISWISGGTKYAD | 5 |
| | SVKGRFTMSRDNAKKQVYLEMNSLKPEDTAVYYCNDMPYGLDTRMDEYAYWGQGTQVTVSS | |
| Bm18A09 | QVQLVESGGGLVQAGGSLRLSCAASGLTGSSYAMGWFRQAPGKEREFVAAISWISGSTKYTD | 9 |
| | SVKDRFTMSRDNAKKQVYLEMNSLKPEDTAVYYCNDMPYGLDTRMDEYVYWGQGTQVTVSS | |
| Re42B03 | QVQLVESGGGLVQAGGSLRLSCAASGLTGSSYAMGWFRQAPGKEREFVAAISWISGGTKYTD | 12 |
| | SVKDRFTMSRDYAKKQVYLEMNSLKPEDTAVYYCNDMPYGLDTRMDEYVYWGQGTQVTVSS | |
| Bm18F11 | QVQLVESGGDLVQAGGSLRLSCAASGLTGSSYAMGWFRQAPGKEREFVAAISWISGDTKYTD | 13 |
| | SVKGRFTMSRDNAKKQVYLEMNSLKPEDTAVYYCNDMPYGLDTRMDEYVYWGQGTQVTVSS | |
| Bm18B05 | QVQLVESGGGLVQPGGSLRLSCAVSGRTESSYAMGWFRRPPGKEREFVAAISWSGGDTKYAD | 20 |
| | FVTDRFTISRDDAKSTVYLQMNSLKPEDTAVYYCNDMPYGLDTRMDEYEYWGQGTQVTVSS | |
| Bm43B02 | QVQLVESGGGLVQAGGSLNLSCVISRRTPSGYAMGWFRQASGKEREFVAAISWIGGGAVYAD | 23 |
| | SVKGRFTMSRDNAKNTVYLQMNSLKPEDTAVYYCNDMPYGLDTTMDDYDYWGQGTLVTVSS | |
| Bm44B11 | QVQLVESGGGLVQAGGSLNLSCVISRRTPSGYAMGWFRQASGKEREFVAAISWIGGGAVYAD | 27 |
| | SVKGRFTMSRDNAKNTVYLQMNSLKPEDTAVYYCNDMPYGLDTTMDDYDYWGQGTQVTVSS | |
| Bm42B11 | QVQLVESGGDLVQAGGSLRLSCSVSGLRVSSYAMGWFRQAPGKEREFVAAISWVGGDTRYAD | 28 |
| | SVKGRFTMSRDNAKKQVYLEMNSLKPEDTAVYYCNDMPYGMETRMDDYASWGQGTQVTVSS | |
| Bm44C09 | QVQLVESGGGLVQAGGSLRLSCAASGLAVSSYAMGWFRQAPGKEREFVAAISWISGGTKYAD | 32 |
| | SVKDRFTMSRDNAKKQVYLEMNSLKPEDTAVYYCNDMPYGLDTRMDDYKYWGQGIQVTVSS | |
| Class B | | |
| Bm17B02 | QVQLVESGGGLVNTGGSLRLSCVVSGHTSEISAMGWYRQAPGKQRELVALITSRSGDTHYSD | 35 |
| | SVKGRFSISRDNAKNTMYLQMNSLNPDDTAVYYCVHNEPGHLYMAQGTQVTVSS | |
| Re42F08 | QVQLVESGGGLVKAGGSLRLSCVVSGHTSEISAMGWYRQAPGKQRELVALITSRSGDTHYSD | 39 |
| | SVKGRFSISRDNAKNTMYLQMNSLNPDDTAVYYCVHNEPGHLYMAQGTQVTVSS | |
| Bm44B04 | QVQLVESGGGLVRTGGSLRLSCSVSGDISSQSAMGWYRQVPGRQRELVAMIHTTKGDTYYAD | 40 |
| | SVKGRFTISRINANNTIYLQMNNLKPEDTAIYYCTHNEPGDLYMGQGTQVTVSS | |
| Bm44G07 | QVQLVQSGGGLVQTGGSLRLSCSVSGDISSQSAMGWYRQVPGRQRELVAMIHTTKGDTYYAD | 44 |
| | SVKDRFTISRINANNTIHLQMNNLKPEDTAIYYCTHNEPGDLYMGQGTQVTVSS | |
| Bm42A09 | QVQLVESGGGLVRTGGSLRLSCSVSGDISSQSAMGWYRQVPGRQRELVAMIHTTKGDTYYAD | 45 |
| | SVKGRFTISRINANNTIYLQMNNLKPEDTAIYYCTHNEPGDLYMGQGTLVTVSS | |
| Class C | | |
| Bm42A03 | QVQLVESGGGLVQPGGSLRLSCVASGFTFSSFAMSWYRQDPGKERKLVAAITNDGGTTFYSD | 49 |
| | LVKGRFTISRDNTKNTVYLQMNNVKPDDAAVYYCMAVRGLYGSNWYDYPFELWGQGTQVTVS | |
| | S | |
| Bm44G10 | QVQLVESGGGLVQPGGSLRLSCVASGFTFSSFAMSWYRQDPGRERKLVAAITNDGGTTFYSD | 53 |
| | LVKGRFTISRDNTKNTVYLQMNNVKPDDAAVYYCMAVRGLYGSNWYDYPFELWGQGTQVTVS | |
| | S | |
| Class D | | |
| Bm45G07 | QVQLVESGGGFVQAGGSLKLSCSVSGNTFITNHMGWFRQPPGKGREFVAGIRWSDGSTFYGD | 54 |
| | SVRGRFTISGDNAKHTLYLQMNSLKPDDTAVYYCYIDSGSDRYYGQGTLVTVSS | |
| Controls | | |
| VHH662 | EVQLVESGGGSVQAGDSLRLSCAASGRANSINWFGWFRQTPGKEREFVAGIRWSDAYTEYAN | 47 |
| | SVKGRFTISRDNAKNTVDLQMDSLKPEDTAVYYCVLDLSTVRYWGQGTQVTVSS | |
| VHH664 | EVQLVESGGGLVQAGGSLRLSCAASGRTYDAMGWLRQAPGKEREFVAAISGSGDDTYYADSV | 48 |
| | KGRFTISKDNAGITMYLQMNSLKPEDTAVYYCATRRGLYYVWDSNDYENWGQGTQVTVSS | |
| Re32D03 | QVQLVESGGGLVQPGGSLRLSCAISGITLDYYAVGWFLQAPGKEREGISCMRNWDGRTVYAP | 64 |
| | SVKGRFTISSDNAKKMVYLEMDNLKSEDTGVYYCAAGPLPPGISCRIPTPLGYDDWGQGTQV | |
| | TVSS | |
| Bm17D12 | QVQLVESGGDLVQPGGSLRLSCAASGFTLDDYVIGWFRQAPGKEREGVACISSISNSDGRAY | 65 |
| | YVDSVKGRFTISRDNAKNTVYLQMNSLKLEDTAVYYCAAERGRIDPDVAGYFLRESYVMDYW | |
| | GNGTLVTVSS | |
| Bm17B11 | QVQLVESGGGLVQPGGSLRLSCAASGFIFSHYWMYWVRQAPGKGLEWVSGINTGGRTTVYAD | 66 |
| | SVRGRFTTSRDNAKNTLYLQMTSLQSEDTAVYYCTALRGDYGKDLTSLDEYEYWGQGTQVTV | |
| | SS | |
| Bm18F01 | QVQLVESGGGLVQPGGSLRLSCAASGFIFSHYWMYWVRQAPGKGLEWVSAINTGGRNTVYAD | 67 |
| | SVKGRFTISRDNAKNTLYLQMNSLKSEDTAVYYCTALRGDYGKDLVSLDEFEYWGQGTQVTV | |
| | SS | |

The present invention relates to a VHH antibody, which is a monovalent heavy chain-only antibody comprising a CDR1 domain, a CDR2 domain and a CDR3 domain linked by framework regions including, but not being limited to, whole VHH antibodies, e.g. native VHH antibodies comprising framework regions derived from camelids, and modified VHH antibodies comprising modified framework regions, VHH antibody fragments and VHH antibody fusion proteins, e.g. a fusion protein with an immunoglobulin or non-immunoglobulin peptide or polypeptide, as long as it shows the properties according to the invention.

There are several methods known in the art for determining the CDR sequences of a given antibody molecule, but there is no standard unequivocal method. Determination of CDR sequences from antibody heavy chain variable regions can be made according to any method known in the art, including, but not limited to, the methods known as KABAT, Chothia, and IMGT. Common determination methods like KABAT would exclude essential parts of the variable regions as a VHH antibody binding site (paratope) often includes residues of the scaffold and outside a narrowly defined CDR region. This is also a difference between nanobodies and traditional antibodies. Other methods of identifying a binding site and CDR sequences of VHH antibodies include the use of custom reference databases utilizing large collection of VHH antibody sequencing data. A selected set of CDRs may include sequences identified by more than one method. CDRs may also be defined through a multiple alignment (with many other VHH antibodies), to identify the hot-spots of variability and relate them to a standard VHH antibody structure. It is also possible to define CDRs by analyzing the structure of the VHH antibody and deciding what is a loop and what is the antibody's scaffold. In some cases, CDR-adjacent residues are also variable, and are therefore included in the CDR definition. According to some embodiments of the present invention, the CDR sequences of the VHH antibodies variable regions are determined using custom reference database, containing sequencing data of ≥10000 VHH antibodies.

The present invention is also directed to a covalent or non-covalent conjugate of a VHH antibody molecule to a non-proteinaceous structure, for example, a labeling group, a capture group such a solid phase-binding group, or an effector group such as a toxin. For example, the heterologous moiety may be from a fluorescence group, biotin, an enzyme such as a peroxidase, phosphatase, or luciferase, a hapten, an affinity tag, or a nucleic acid such as an oligonucleotide.

The VHH antibody of the present invention is particularly a monoclonal VHH antibody characterized by a specific amino acid sequence. The VHH antibody may be produced in a prokaryotic host cell, a yeast cell or a mammalian cell. In certain embodiments, the VHH antibody is non-glycosylated. In some embodiments, a glycosylation site in a parent VHH antibody sequence is mutated to eliminate a predicted glycosylation. In particular embodiments, the mutation comprises substitution of an Asparagine (Asn, N) residue in an N-glycosylation site to prevent potential glycosylation of the VHH antibody. In more particular embodiments, an Asn residue is substituted to prevent or eliminate glycosylation in a VHH antibody selected from: Bm43B02 (at position 19 of SEQ ID NO. 23); Bm44B11 (at position 19 of SEQ ID NO. 27); Bm44B04 (at position 76 of SEQ ID NO. 40); Bm44G07 (at position 76 of SEQ ID NO. 44); and Bm42A09 (at position 76 of SEQ ID NO. 45).

In certain embodiments, the VHH antibody is glycosylated, wherein a carbohydrate structure may be derived from a glycosylation site introduced into the VHH sequence and/or from a fusion partner.

A VHH antibody according to the present invention is characterized by (i) a CDR3 sequence, (ii) a combination of a CDR1 sequence, a CDR2 sequence, and a CDR3 sequence, (iii) by a complete VHH sequence, or (iv) by competition with a specific reference antibody. Specific CDR and VHH sequences are provided in the Tables, Figures and the Sequence Listing.

According to the present invention, sequences related to the above sequences are encompassed. These related sequences are defined by having a minimum identity to a specifically indicated amino acid sequence, e.g., a CDR or VHH sequence. This identity is indicated over the whole length of the respective reference sequence and may be determined by using well-known algorithms such as BLAST.

In particular embodiments, a related CDR3 sequence has an identity of at least 80% or at least 90% or at least 95% to a specifically indicated CDR3 sequence, e.g., a substitution of 1, 2, or 3 amino acids.

In particular embodiments, a related combination of a CDR1 sequence, a CDR2 sequence, and a CDR3 sequence has an identity of at least 80% or at least 90% or at least 95% to a specifically indicated combination of a CDR1 sequence, a CDR2 sequence, and a CDR3 sequence, e.g., a substitution of 1, 2, 3, 4, 5 or 6 amino acids by different amino acids.

In particular embodiments, a related VHH sequence has an identity of least 70%, at least 80%, at least 90%, at least 95% or at least 99% to a VHH sequence, e.g., a substitution of 1, 2, 3, 4, 5 or up to 20 amino acids.

Further, the invention refers to a VHH antibody, which competes with a specific VHH antibody disclosed herein for the binding to a human IL-17 polypeptide. In certain embodiments, a competing VHH antibody binds the same or an overlapping epitope on the human IL-17 polypeptide. For example, the invention refers to a VHH antibody, which competes either with a reference antibody, e.g., VHH antibody Re42H11 or with VHH antibody Bm17B02. Competition may be determined by label-free biolayer interferometry performed as a cross-competition or epitope binning assay using a label-free detection system, e.g., an Octet® system from Sartorius, according to the manufacturer's instructions.

In particular embodiments, at least one amino acid of a reference sequence, including an amino acid in a CDR1, CDR2 or CDR3 sequence and/or an amino acid in a framework region, is replaced by another amino acid, while preserving structural integrity and epitope-binding of the VHH antibody. These exchanges can be conservative (i.e., by a similar amino acid) or non-conservative.

In further particular embodiments, at least one amino acid of a reference sequence, including an amino acid in a CDR1, CDR2 or CDR3 sequence and/or an amino acid in a framework region, is replaced by a conservative amino acid substitution, i.e. a substitution of an amino acid by another amino acid with similar biochemical properties, for example a substitution of an aliphatic amino acid, e.g. Gly, Ala, Val, Leu, or Ile, for another aliphatic amino acid; a substitution of a basic amino acid, e.g. His, Lys or Arg, against another basic amino acid or against Met; a substitution of an acidic amino acid or an amide thereof, e.g., Asp, Glu, Asn or Gln, against another acidic amino acid or an amide thereof; a substitution of an aromatic amino acid, e.g., Phe, Tyr or Trp, against another aromatic amino acid.

In further particular embodiments, the VHH antibody is selected from antibody Re42B04a comprising a VHH sequence as shown in SEQ ID NO. 19 or a VHH antibody, which is a variant thereof. In certain embodiments, 1, 2, 3, 4, 5, 6, 7, 8, 9 or 10 amino acids of SEQ ID NO. 19 are replaced by another amino acid.

In further particular embodiments, the VHH antibody is selected from antibody R242B04 comprising a VHH sequence as shown in SEQ ID NO. 15 or a VHH antibody, which is a variant thereof. In certain embodiments, 1, 2, 3, 4, 5, 6, 7, 8, 9 or 10 amino acids of SEQ ID NO. 15 are replaced by another amino acid.

In further particular embodiments, the VHH antibody is selected from antibody Re42F08 comprising a VHH sequence as shown in SEQ ID NO 39 or a VHH antibody, which is a variant thereof. In certain embodiments, 1, 2, 3, 4, 5, 6, 7, 8, 9 or 10 amino acids of SEQ ID NO. 39 are replaced by another amino acid.

In further particular embodiments, the VHH antibody is selected from antibody Bm17B02 comprising a VHH sequence as shown in SEQ ID NO. 35 or a VHH antibody, which is a variant thereof. In certain embodiments, 1, 2, 3, 4, 5, 6, 7, 8, 9 or 10 amino acids of SEQ ID NO. 35 are replaced by another amino acid.

The invention provides four classes of VHH antibodies based on sequence similarity (80% or higher sequence identity for VHHH antibodies of the same class) and on data gained from crystal structures of complexes of the VHH antibodies and human IL-17. These crystal structures have been used to define the amino acid residues in the VHH sequence that mostly contribute to binding to IL-17 and neutralizing its binding to the receptor. Each VHH antibody in a specific class has at least 80% sequence identity to other members of the same class.

Further, the present invention relates to a nucleic acid molecule, e.g., a DNA molecule, encoding a VHH as indicated above, or a vector, comprising said nucleic acid molecule as indicated above in operative linkage with an expression control sequence, particularly with a heterologous expression control sequence. Furthermore, the invention relates to a cell comprising a nucleic acid molecule or a vector as described above. Vectors for the recombinant production of VHH antibodies are well-known in the art. In certain embodiments, the vector is an extrachromosomal vector. In other embodiments, the vector is a vector for genomic integration. The cell may be a known host cell for producing antibodies or antibody fragments, e.g., a prokaryotic cell such as an E. coli or a Bacillus sp. cell, a yeast cell, particularly a Pichia yeast cell, an insect cell or a mammalian cell, e.g., a CHO cell, or a plant cell. In certain embodiments, the cell comprises the nucleic acid or the vector extrachromosomally. In other embodiments, the cell comprises the nucleic acid or the vector integrated into the genome, e.g., as a genomically integrated expression cassette.

Still a further aspect of the present invention is a method of recombinantly producing a VHH antibody by growing a cell as described above in a culture medium and obtaining the VHH antibody from the cell or the culture medium. Suitable culture media and culture conditions are well known in the art.

### Binding to Human IL17

The VHH antibodies of the present invention bind to a human IL-17 polypeptide. The inventors have identified VHH antibodies, which bind with high affinity to human IL-17 polypeptides and cross-react with (i) a human IL-17A homodimer, (ii) and human IL-17F homodimer as shown in Table 3.

In the context of the present disclosure the term “human IL-17”. This term encompasses a plurality of different human IL-17 family members, or isoforms, including but not limited to:
IL-17A (UniProt Accession No. Q16552),
IL-17B (UniProt Accession No. Q9UHF),
IL-17C (UniProt Accession No. Q9P0M4),
IL-17D (UniProt Accession No. Q8TAD2),
IL-17E (UniProt Accession No. Q9H293),
IL-17F (UniProt Accession No. Q96PD4).

The term “human IL-17” encompasses human IL-17A (UniProt Accession No. Q16552) particularly in the form of a homodimer comprising two IL-17A units, IL-17F (UniProt Accession No. Q96PD4) particularly in the form of a homodimer comprising 2 IL-17F units, and IL-17AF in the form of a heterodimer comprising one IL-17A unit and one IL-17F unit.

It should be noted, however, that the term “human IL-17” also encompasses naturally occurring variants of human IL-17 polypeptides and genetically modified constructs as described herein. The inventors have performed their selection and binding experiments with genetically modified IL-17A, IL-17F and IL-17A/F constructs as follows:
An IL-17A homodimer that comprises amino acid residues 40-155 of human IL-17A (UniProt Q16552). This corresponds to the portion that is well-ordered in crystal structures. The first five amino acids (GSEDS) are spacer residues. Further, it comprises two point mutations: N68D (eliminates an N-glycosylation site) and C192S (eliminates an unpaired cysteine that would otherwise cause problems in recombinant expression). The amino acid sequence of this polypeptide is shown in SEQ ID NO. 1.
An IL-17F homodimer that comprises amino acid residues 39-163 of human IL-17F (UniProt Q96PD4). N83D mutation to eliminate an N-glycosylation site. The first five residues (GSEGE) are a linker. The amino acid sequence of this polypeptide is shown in SEQ ID NO. 2.
An IL17A/F heterodimer that consists of two polypeptides. The IL-17A unit comprises amino acid residues 40-155 of human IL-17A (UniProt Q16552) with 5 spacer residues (GSEDS) in front and a N68D mutation. The amino acid sequence of this polypeptide is shown in SEQ ID NO. 3. The IL-17F unit comprises amino acid residues 39-163 of human IL-17F (UniProt Q96PD4) with 5 spacer residues (GSEDS) in front and N83D and C137S mutations. The amino acid sequence of this polypeptide is shown in SEQ ID NO. 4.

The above bacterially expressed IL-17A, IL-17F, and IL-17AF proteins were used during the initial immunization, selection, characterization, and crystallization.

For immunization and selection of second generation VHH antibodies and for their characterization, mammalian expressed versions of IL-17A and IL-17F, which lack the indicated modification were used as follows:
Mature human IL-17A in mammalian expression (UniProt Q16552). The amino acid sequence of this polypeptide is shown in SEQ ID NO. 77.
Mature human IL-17F in mammalian expression (UniProt Q96PD4). The amino acid sequence of this polypeptide is shown in SEQ ID NO. 78.

In certain embodiments, the VHH antibody of the present invention binds to (i) a human IL-17A homodimer, (ii) a human IL-17F homodimer and (iii) a human IL17A/F heterodimer wherein the binding affinity expressed as dissociation constant KD to each of (i), (ii), and (iii) is about 1 nM or less, about 100 μM or less, about 50 μM or less, about 20 μM or less or about 10 μM or less. The binding affinity may be determined as described herein in detail in the Examples and Figures, using e.g., the polypeptides of SEQ: ID NO: 1-4 and 77-78, as described above.

### IL-17 Neutralization

The VHH antibodies of the present invention are capable of neutralizing the binding of human IL-17 dimer to a human IL-17 receptor and are capable of preventing or inhibiting activation of the receptor. The inventors have identified VHH antibodies, which cross-neutralize (i) a human IL-17A homodimer, (ii) a human IL-17F homodimer and (iii) a human IL17A/F heterodimer as shown in Table 3.

In certain embodiments, the VHH antibody of the present invention cross-neutralizes the binding of a human IL-17 polypeptide, particularly (i) a human IL-17A homodimer, (ii) a human IL-17F homodimer and (iii) a human IL17A/F heterodimer to a human IL-17 receptor The neutralization potency may be determined as described herein in detail in the Examples.

### Stability

For the intended therapeutic application, the anti-IL-17 VHH antibodies should not only be highly potent in interleukin neutralization, but also, they should be developable as biological drugs. This includes that they are stable enough to survive a lengthy, large-scale production process as well as transportation and storage (ideally for years in liquid, semi-liquid or solid formulations) without aggregation or loss of activity.

A good predictor for stability is thermostability, which can be measured, e.g., by thermal shift assays or specifically by differential scanning fluorimetry. The present inventors have identified several thermostable or hyperthermostable VHH antibodies as shown in Table 3.

In a particular embodiment, the invention relates to a VHH antibody, which is stable, particularly thermostable, or hyperthermostable. Preferably, the VHH antibody has a melting point (melting temperature) of at least about 65° C., of at least about 80° C., of at least 90° C. or of at least about 95° C., and/or an aggregation temperature of at least about 50° C., of at least about 60° C., of at least 70° C. or of at least about 80° C. when measured under non-reducing conditions. Melting and aggregation temperatures are determined as described herein.

In a particular embodiment, the invention relates to a VHH antibody, which is stable, particularly thermostable or hyperthermostable. Specifically, the VHH antibody has a melting point (melting temperature) of at least about 65° C., of at least about 80° C., of at least 90° C. or of at least about 95° C., and/or an aggregation temperature of at least about 50° C., of at least about 60° C., of at least 70° C. or of at least about 80° C. when measured under non-reducing conditions. Melting and aggregation temperatures are determined as described herein. According to some embodiments, a stable VHH antibody has a melting point (melting temperature) of at least about 65° C. and/or having an aggregation temperature of at least 50° C., and an hyperthermostable VHH antibody has a melting point (melting temperature) of at least 95° C., and/or an aggregation temperature of at least about 80° C. when measured under non-reducing conditions. It should be noted however that the outcome of a stability measurement depends on the conditions. Aggregation, for example, is favored by a high protein concentration, by a pH close to the isoelectric point of the protein. The stability is also influenced by the buffer composition and additives used with the tested ingredient.

### Sets of VHH Antibodies

In a further aspect, the present invention relates to a set comprising at least 2, 3, 4 or more of the above VHH antibodies. In such a set, the individual VHH antibodies are present in suitable molar ratios. Typically, the molar ratios are in the range of about 2:1 to about 1:2, particularly about 1.5:1 to about 1:1.5, even more particularly about 1:1. In certain embodiments, the VHH antibody set may comprise a single composition wherein the VHH antibodies in said set consist of a predetermined number of different species of VHH antibodies as described above. The VHH antibody set may comprise a plurality of compositions each comprising a different species of VHH antibody as described above. The set of the present invention may be free from other VHH antibodies.

### Monovalent and Multivalent VHH Antibodies

In certain embodiments, the VHH antibody of the present invention is in a monovalent format, i.e., it has a single binding site for a human IL-17 polypeptide. In these embodiments, the VHH antibody may be present as such or covalently or non-covalently attached to a heterologous moiety, e.g., a peptidic or non-peptidic moiety.

In further embodiments, the VHH antibody of the present invention is in in a multimeric, e.g., dimeric, or trimeric format. In these embodiments, several VHH antibody units may be covalently or non-covalently attached to each other together via a linker and/or a multimerization, e.g., dimerization or trimerization moiety.

In certain embodiments, the VHH antibody is a homodimeric VHH antibody, wherein a VHH antibody unit is covalently attached to a dimerization moiety, e.g., an immunoglobulin Fc fragment.

In certain embodiments, the VHH antibody is a heterodimeric VHH antibody, particularly a covalently linked VHH heterodimer comprising a first VHH antibody and a second VHH antibody wherein the first VHH antibody and the second VHH antibody bind to different epitopes on IL-17 or wherein the first VHH antibody binds to IL-17 and the second VHH antibody binds to a different target.

### Production of VHH Antibodies

VHH antibodies including monomeric and multimeric VHH antibodies may be produced as described in WO 2022/023483 and WO 2022/023484, the contents of which are herein incorporated by reference, or by other methods well known in the art.

A VHH antibody may be recombinantly produced in a suitable host cell, e.g., in a prokaryotic or eukaryotic host cell or host organism. For this purpose, a nucleic acid molecule encoding the VHH antibody is introduced into the host cell or host organism and expressed in the host cell or host organism. The nucleic acid molecule may encode a monomeric VHH antibody or a subunit of a multimeric VHH antibody.

In a particular embodiment, the VHH antibody is recombinantly produced in a bacterium, e.g., E. coli or Bacillus. For example, expression in a bacterium may involve cytoplasmic and/or periplasmic expression and purification of the VHH antibody from the host cell, or secretory expression and purification of the VHH antibody from the culture medium. In certain embodiments, the nucleic acid sequence encoding the VHH antibody is fused to at least one sequence directing the expression to the periplasm and/or into the culture medium.

In a further particular embodiment, the VHH antibody is recombinantly produced in a eukaryotic host cell or host organism, preferably in yeast, e.g., in Pichia pastoris, Saccharomyces cerevisiae, or Hansenula polymorpha, or in an animal cell, particularly in a mammalian, e.g., human or a hamster cell. For example, expression in a eukaryotic host cell or host organism, e.g., yeast may involve cytoplasmic and/or periplasmic expression and purification of the VHH antibody from the host cell, or preferably secretion from the host cell and purification of the VHH antibody from the culture medium. In certain embodiments, the nucleic acid sequence encoding the VHH antibody is fused to at least one sequence directing the expression into the culture medium.

VHH antibodies may be produced using any method known in the art for producing proteins, antibodies and nanobodies. Typically, the VHH antibodies are produced recombinantly in a prokaryotic or eukaryotic host cell system or host organism, such as a bacterium, a yeast, a plant cell or in a mammalian cell. According to some embodiments, the VHH antibodies are produced recombinantly in Pichia pastoris.

In yet more particular embodiment, the VHH antibody is produced using the method illustrated in FIGS. 20A and 20B.

### Therapeutic Applications and Methods of Use

Still a further aspect of the present invention is the use of a VHH antibody as described above in medicine, particularly for therapeutic and/or in vitro or vivo diagnostic use. In certain embodiments, the VHH antibody is used in human medicine.

The VHH antibody of the present invention is useful in the prevention or treatment of a disorder caused by and/or associated with an overactivity of IL-17, particularly associated with an overactivity of IL-17A and/or IL-17F.

In certain embodiments, the VHH antibody is useful in the prevention or treatment of an inflammatory and/or immune-related disorder, e.g., an inflammatory and/or immune-related skin disorder.

Exemplary disorders are asthma, psoriasis, e.g., plaque psoriasis, arthritis, e.g., rheumatoid arthritis or psoriatic arthritis, Hidradenitis suppurativa, inflammatory bowel disease (Crohn's disease, ulcerative colitis), multiple sclerosis, skin cancer, ankylosing spondylitis, uveitis, atopic dermatitis, Graft versus Host Disease, Alzheimer's disease, fatty liver disease, COVID-19, sepsis, ischemic stroke, Parkinson's disease, influenza virus infection.

In some embodiments, the disorder is plaque psoriasis. In specific embodiments, the disorder is mild or moderate plaque psoriasis.

In therapeutic applications, the VHH antibody is administered in an effective amount to a subject in need thereof, particularly to a human subject. The dose will depend on the specific type of agent, e.g., monovalent VHH antibody or multimeric VHH antibody, the type of disease, and the route of administration.

Typically, the VHH antibody is administered as a pharmaceutical composition comprising the active agent and a pharmaceutically acceptable carrier or excipient. Examples of suitable carriers and excipients for formulating antibodies or antibody fragments are well-known in the art.

The present invention further provides methods of treating diseases and disorders associated with overexpression or overactivity of human IL-17, comprising administering to a subject in need of such treatment a pharmaceutical composition comprising an effective amount of a VHH antibody that neutralizes IL-17 receptor activation, and a pharmaceutically acceptable carrier, thereby treating the disease or disorder.

In certain embodiments, disease or disorder is an inflammatory and/or immune-related disorder. According to more particular embodiments, the disease or disorder is an inflammatory and/or immune-related skin disorder.

Exemplary disorders treatable with the VHH antibodies of the present invention are psoriasis, e.g., plaque psoriasis, asthma, arthritis, e.g., rheumatoid arthritis or psoriatic arthritis, Hidradenitis suppurativa, inflammatory bowel disease (Crohn's disease, ulcerative colitis), multiple sclerosis, skin cancer, ankylosing spondylitis, uveitis, atopic dermatitis, Graft versus Host Disease, Alzheimer's disease, fatty liver disease, COVID-19, sepsis, ischemic stroke, Parkinson's disease, influenza virus infection.

In some embodiments, the disorder is plaque psoriasis. In specific embodiments, the disorder is mild or moderate plaque psoriasis.

According to some embodiments, the pharmaceutical composition comprising an effective amount of a VHH antibody that neutralize IL-17 is administered locally to a site of a subject in need of such treatment, and a pharmaceutically acceptable carrier, thereby treating the disease or disorder.

The terms “administering locally”, “local administration” or “dermal administration” mean that the VHH antibody is not administered systematically. The terms include in particular topical application on the surface of the skin. Local administration also encompasses administration of the composition by injection, particularly by intraepidermal injection and/or intradermal injection and/or subdermal injection and/or subcutaneous injection and/or micro-injection.

The compositions of the present invention are suitable, according to some embodiments, for administration by topical or injectable routes of administration (in particular intraepidermal and/or intradermal and/or micro-injection, and/or by systemic injection, e.g., subcutaneous injection.

The methods of the present invention include administering locally the pharmaceutical composition to a defined area of skin.

The treatment period is ideally of sufficient time to provide an improvement in the disease or disorder treated. The treatment period can be at least 1 week, and in some embodiments the treatment period can last about 4 weeks, 8 weeks, or 12 weeks. In certain embodiments, the treatment period will extend over multiple months (i.e., 3-12 months) or multiple years. In one embodiment, the pharmaceutical composition is applied at least once a day during a treatment period of at least 4 weeks, 8 weeks, or 12 weeks. In one embodiment, the pharmaceutical composition is applied twice a day during a treatment period of at least 4 weeks, 8 weeks, or 12 weeks. Alternatively, the pharmaceutical composition is administered once every other day, once every three days, once a week for a week, a month, or as long as an improvement is achieved.

While the subject for the pharmaceutical methods may be of any suitable age, the subject is, in some embodiments, a child, an adult or a geriatric subject.

The pharmaceutical composition suitable for dermal administration may be used in combination with mechanical devices, such as massage-roller devices having a mechanical and rubbing action to facilitate penetration of the active agent, or wave-emitting systems (light, low frequencies, infrared frequencies, etc.) which activate the response of the skin. Additional means that may be used or combined with the other administration routs are patches and microneedles.

The pharmaceutical compositions of the invention suitable for injection, in particular for intraepidermal and/or intradermal and/or subcutaneous injection and/or micro-injection, can be injected by a device comprising a needle or microneedle or by a needle-free injection device. Such devices are well known in mesotherapy. Alternatively, the pharmaceutical compositions can be administered by means of iontophoresis directly to achieve a greater penetration of the active agent.

The terms “treat,” “treating,” and “treatment” are all intended to refer to an amelioration or reversal of at least one measurable physical parameter related to the amendable disease or disorder.

The term “treatment” or “treating” may be used interchangeably herein refers to inhibiting, preventing or arresting the development of a disease or disorder and/or causing the reduction, remission, or regression of a disease or disorder.

The phrase “inflammatory disease or disorder” used herein refers to a disease, condition or disorder associated with inflammation. The term “inflammation” as used herein refers the process by which a subject's immune system coordinates a response to tissue damage, infection, antigenic challenge, etc. Inflammation may be associated with an increased blood supply to the tissue, increased capillary permeability in the tissue and/or increased leukocyte migration to the tissue.

According to some embodiments of the present invention the inflammatory disease or disorder is an autoimmune disease or disorder. The autoimmune disease or disorder can be, but not limited to psoriasis, arthritis, Systemic lupus erythematosus (Lupus, SLE), multiple sclerosis, and inflammatory bowel diseases.

### Pharmaceutical Compositions

The VHH antibodies of the present invention may be formulated in pharmaceutical compositions of any form, according to the intended use. Optional forms of pharmaceutical compositions according to the present invention include liquid, semi liquid, solid and semi solid formulations.

According to some embodiments, the VHH antibodies are formulated for topical administration, for example as cream, paste, gel, hydrogel, ointment, lotion, and emulsion.

According to some embodiments, the pharmaceutical composition further comprises one or more excipient, carrier, or buffer. According to some embodiments, the pharmaceutical composition comprises an excipient selected from the group consisting of emulsifying agents, pH buffering agents, preservatives, chelating agents, tonicity agents, humectants, antioxidants, and gelling agents.

According to additional embodiments, the pharmaceutical composition is in the form selected from the group consisting of a solution, emulsion, nanoemulsion, suspension, lipid nanoparticles (e.g., liposomes), microparticles, ointment, cream, lotion, paste, gel, hydrogel, spray, powder, stick, and a patch. Each possibility represents a separate embodiment of the invention.

The present invention provides pharmaceutical compositions comprising as an active agent at least one VHH antibody that neutralize IL-17 and a pharmaceutically acceptable carrier, diluent, or excipient.

The term “pharmaceutical composition” as used herein refers to a composition suitable for treating a disease or disorder, caused by and/or associated with an overactivity of IL-17, particularly associated with an overactivity of IL-17A and/or IL-17F. Particularly, the pharmaceutical compositions are useful in the prevention or treatment of an inflammatory and/or immune-related disorder, e.g., an inflammatory and/or immune-related skin disorder. Exemplary disorders are asthma, psoriasis, e.g., plaque psoriasis, arthritis, e.g., rheumatoid arthritis or psoriatic arthritis, Hidradenitis suppurativa, inflammatory bowel disease (Crohn's disease, ulcerative colitis), multiple sclerosis, skin cancer, ankylosing spondylitis, uveitis, atopic dermatitis, Graft versus Host Disease, Alzheimer's disease, fatty liver disease, COVID-19, sepsis, ischemic stroke, Parkinson's disease, influenza virus infection.

The pharmaceutical compositions further comprise a pharmaceutically acceptable carrier.

The term “pharmaceutical acceptable” as used herein mean approved by a regulatory agency of the Federal or a state government or listed in the U.S. Pharmacopeia or other generally recognized pharmacopeia for use in humans. The constituents of the pharmaceutical compositions of the present invention are all pharmaceutically acceptable agents.

The term “carrier” refers to a diluent, adjuvant, excipient, or vehicle with which the compound is administered. The carriers can be liquids, preferably sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents.

The composition, if desired, can also contain minor amounts of emulsifying agents, anionic emulsifiers, synthetic polymers, lipid base excipients, lecithin or pH buffering agents such as acetates, citrates or phosphates. Preservative such as benzyl alcohol or methyl parabens; chelating agents such as ethylenediaminetetraacetic acid; and agents for the adjustment of tonicity such as sodium chloride or dextrose, are also envisioned.

Other optional excipients include, but are not limited to, humectants such as water soluble liquid polyols, e.g., glycerin, propylene glycol, hexylene glycol, butylene glycol, pentylene glycol, dipropylene glycol, and mixtures thereof; antioxidants such as glycolic acid, citric acid, lactic acid, malic acid, mandelic acid, ascorbic acid, sodium bisulfite, vitamin E and derivatives thereof; wetting agents; suspending agents; gelling agents; skin emollients and skin moisturizers; antimicrobial (e.g., antibacterial) agents; antifungals; analgesics; UV absorbers; wound healing promoters; growth factors; reactive oxygen species; anti-inflammatory agents; vitamins such as vitamin C, vitamin B, and derivatives thereof; nutrients such as thiamin, riboflavin, niacin, pantothenates, pyridoxine, folic acid, cobalamin, biotin, choline, inositol, carnitine, etc.; amino acids and their derivatives such as alanine, arginine, asparagine, aspartic acid, carnitine, citrulline, cysteine, dimethylglycine, gamma-aminobutyric acid, glutamic acid, glutamine, glutathione, glycine, histidine, isoleucine, leucine, lysine, methionine, ornithine, phenylalanine, praline, serine, taurine, threonine, tryptophan, tyrosine, valine; minerals such as boron, calcium, chromium, cobalt, copper, fluoride, germanium, iodine, iron, lithium, magnesium, manganese, molybdenum, phosphorus, potassium, selenium, silicon, sodium, sulfur, vanadium, zinc; herbal extracts such as green tea, algae, aloe, etc.; retinoids; flavonoids; and mixtures thereof.

The compositions can be in the form of a solution, emulsion (e.g., oil-in-water, water-in-oil-in-water, water-in-oil or oil-in-water-in-oil), nanoemulsion, suspension, microparticles, oil, ointment, cream, lotion, paste, gel, hydrogel, spray, powder, stick, or combinations thereof.

The pharmaceutical compositions of the present invention may be formulated for sustained release, slow release, or delayed release, using methods and ingredients well known in the art. Depot formulations of the VHH antibodies of the present invention are therefore included within its scope.

In some embodiments, a tape or other support structure can be applied to the skin. In some embodiments, the composition is applied to the skin before the tape is placed. In that situation, the tape may be porous or not.

In some embodiments, the tape is a polymer matrix or gel that permits contact of the composition with the skin when the composition is applied over the tape. Such a tape, also designated a patch or transdermal patch, has the added advantage of providing controlled delivery of a compound to the body. Transdermal patches can be made by dissolving or dispersing the compound in the proper medium and then applying it to the tape. Absorption enhancers can also be used to increase the flux of the compound across the skin. The rate of such flux can be controlled by the polymer matrix or gel and optionally by a rate controlling membrane. In one embodiment, the polymer matrix can be hyaluronic acid which has the property of trapping water and forming a gel.

For topical application, the composition can be formulated in the form of an ointment containing the VHH antibody dissolved or suspended in appropriate carriers. Such carriers include, but are not limited to, mineral oils, liquid vaseline, white vaseline, propylene glycol, polyoxyethylene, polyoxypropylene, emulsifier wax, and water. Alternatively, it can be formulated as a cream or lotion containing the VHH antibody dissolved or suspended in appropriate carriers. Such carriers include, but are not limited to, mineral oils, sorbitan monostearate, polysorbate 60, cetyl ester wax, cetearyl alcohol, 2-octyldodecanol, benzyl alcohol and water.

For injection, the pharmaceutical composition can be formulated as a liquid such as a solution, emulsion, suspension; or as a gel. According to preferred embodiments, the pharmaceutical composition is a sterile solution or suspension. Acceptable solvents and carriers include, but are not limited to, water, Ringer's solution and isotonic sodium chloride. In addition, sterile oils are often used as a solvent. To this end, any oil may be used, such as mono- and diglycerides. Fatty acids like oleic acid and its glyceride derivatives are also used for preparing injectable compositions, as are pharmaceutically acceptable natural oils like olive oil, castor oil, and in particular their polyoxyethylenated forms. These oily solutions may comprise suspending agents or diluents like carboxymethyl cellulose for the formulation of emulsions and suspensions. Surfactants like Tweens and emulsifiers may also be included. Aqueous or oleaginous suspensions may be formulated by methods well known to one of skill in the art by using wetting agents or dispersants and suspending agents.

As used herein, “microparticles” means polymer or combinations of polymers made into bodies of various sizes. The microparticles can be in any shape, although they are often in substantially spherical shape, in which case the microparticles are referred to as “microspheres” or “microbeads”. Incorporation of the active agent into the microparticles can be accomplished by mixing dry microparticles with solutions of the active agent in an aqueous or hydro-organic solution. Before injection or being composed into an injectable composition, the microspheres are sterilized.

To prepare the pharmaceutical compositions of the present invention, a variety of techniques may be employed. For example, the active agent may be generally incorporated into an acceptable carrier in the manner that is usual for the preparation of pharmaceutical products. Thus, the active agent may first be dissolved or dispersed in a portion of the water or another solvent or liquid to be incorporated into the acceptable carrier. The preferred compositions for use in this manufacturing approach are oil-in-water, water-in-oil, or water-in-oil-in-water emulsions.

In some embodiments, the active agent, with or without excipients, are maintained in a separate state from the carrier, for example, as a dry powder. The intermixing of the desired amount of the active agent with the desired amount of the carrier, performed by the subject immediately prior to application of the pharmaceutical composition, ensures that the active agent will retain its maximum efficacy, and will also permit the potency of the composition to be tailored to the individual needs of the subject. The resulting pharmaceutical composition may be then applied to the skin.

According to further embodiments, the pharmaceutical composition is administered topically. According to certain embodiments, the pharmaceutical composition is administered topically, said composition is in the form of an ointment, cream, lotion, paste, gel, hydrogel, spray, powder, stick, or a patch.

Depending on the stage and the severity of the disorder, the pharmaceutical composition may be administered once or several times during the course of the disorder. For example, it may be administered once or several times daily, each second day, two times weekly, weekly, monthly or every few months, for a suitable period of time.

In certain embodiments, the pharmaceutical composition is administered parenterally, e.g., by subcutaneous, intramuscular or intravenous injection or by infusion. In certain embodiments, the pharmaceutical composition is administered by injection. According to some embodiments, the injection is intraepidermal injection, intradermal injection, subcutaneous injection, micro-injection, or any combination thereof. According to certain embodiments, the pharmaceutical composition administered by injection is in the form of a solution, emulsion suspension, or microparticles.

In further embodiments, the pharmaceutical composition is administered locally, e.g., topically, orally, nasally or intrapulmonary, for example by inhalation as an aerosol.

The VHH antibody may be administered alone or together with a further active agent or treatment, particularly with a further agent that is useful in the prevention and/or treatment of a disorder caused by and/or associated with IL-17 overactivity, particularly associated with an overactivity of IL-17A and/or IL-17F.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of a disease or disorder of the subject being treated.

The term “effective amount” is that amount of the active agent which is sufficient to provide a beneficial effect to the subject to which the composition is administered.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to levels of the active ingredient which are sufficient to achieve the minimal effective concentration (MEC). The MEC will vary for each preparation but can be estimated from in vitro data. Dosages necessary to achieve the MEC will depend on individual characteristics and route of administration. Detection assays can be used to determine plasma or tissue concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the subject being treated, the severity of the affliction, the manner of administration, the judgment of the prescribing physician, etc.

Thus, compositions and/or articles of some embodiments of the invention may, if desired, be presented in a pack or dispenser device, such as an FDA approved kit, which may contain one or more unit-dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container (e.g., lyophilized vial), and labeled for treatment of an indicated condition, as is further detailed above.

in some embodiments, the VHH antibodies and pharmaceutical compositions comprising them are administered to subjects suffering from psoriasis, particularly plaque psoriasis, more particularly mild to moderate plaque psoriasis, as a single treatment or as a treatment regimen that include at least one additional agent or treatment for psoriasis or at least one symptom associated with this disease. According to specific embodiments, the additional treatment includes steroids.

In certain embodiments, the further active agent is an active agent that is useful in the prevention and/or treatment of an exemplary disorder listed above.

The present invention provides kits comprising the compositions of the present invention suitable for injection and a syringe or an injection device.

### Diagnostic Applications

Diagnostic applications include in vitro methods wherein a VHH antibody is used for detecting IL-17 in a sample, e.g., in a body fluid such as saliva, blood, serum or plasma, stool samples or in a tissue or biopsy sample. Diagnostic applications further include in vivo methods wherein a VHH antibody is used for detecting IL-17 in a subject, particularly in a human patient. For diagnostic applications the VHH antibody may carry a label for direct detection or used in combination with secondary detection reagents, e.g., antibodies, including conventional antibodies or VHH antibodies, for indirect detection according to established techniques in the art.

### Evaluating the Activity and Safety of the VHH Antibodies for Use in Therapy

The VHH antibodies and sets of VHH antibodies of the present invention may be tested in vitro, in vivo and ex vivo for their activity in IL-17 related disease models.

In vitro disease models for psoriasis and atopic dermatitis were reviewed in Sarama et al., 2022.

Some of the animal models utilizes animal or human skin. As the homology between the amino acid sequence of human and pig IL-17 is relatively high (73%) it is possible to evaluate the VHH antibodies in pig skin models. Human skin models, e.g., of psoriasis, utilizing cultures of primary human epidermal keratinocytes isolated from neonatal foreskin, are also used to evaluate the potential therapeutic effects of the VHH antibodies. The effect of the VHH antibodies of the present invention on factors regulating psoriatic keratinocytes (Zhou, X. et al. 2022) may also be tested.

As a nonlimitative example, an in vivo study in SCID/Beige mice transplanted with human xenograft (Keren et al., 2018) may be used to test several toxicology parameters. The transplanted mice are considered a suitable in vivo model as normal human skin is engrafted into SCID mouse and disease is induced by injection of IL-2 activated PBMC's from human psoriatic patients. Blocking the cascade downstream of IL-17 by the VHH antibodies of the invention, is determined by improvement of the marker's level (such as S100A7, Ki-67, beta-defensin-2) and skin appearance as judged by H&E staining or qRT-PCR.

In this model healthy human skin pieces are transplanted to SCID mice at day 0. Peripheral Blood Mononuclear Cells (PBMCs) from psoriatic patients' blood are isolated and cultured in the presence of a high dose of IL-2 for 14 days to activate allogeneic T-cells expressing high levels of NK cells receptors. These PBMCs are injected to the mice on day 28 following the engraftment, generating psoriatic-like lesions which develop within the transplants, displaying almost all the key features of human psoriasis. These features include thickening of the epidermis, the presence of immune cells, edema, expanded blood vessels, and increased keratinocyte proliferation. Importantly, these psoriasis-like lesions respond to commonly used anti-psoriatic treatments. This highlights the significant relevance of this pre-clinical model for psoriasis research (Keren et al, 2018). Treatments with VHH antibodies or with controls are done on day 42 while skin harvest and analysis is performed on day 56.

Pre-clinical safety studies include, for example (i) human tissue cross reactivity (TCR) study (ii) pharmacokinetic/biodistribution (PK/BD) study in rats and (ii) a GLP toxicology study in mini pigs.

A GLP toxicology study may be used to confirm an acceptable safety profile of the tested VHH antibody. The currently accepted toxicology animal model for mAbs against psoriasis is performed in non-human primates (NHP) as described for example in Kolbinger et al., 2022. Yet the mini pigs' model used for the VHH antibodies of the present invention is more suitable for the following reasons: 1. VHH antibodies are administered topically or ID and not systemically like mAbs, hence, skin vascularization, structure and neurology structure that is similar in mini pig and human is more relevant. 2. VHH antibodies are cleared from the blood within an hour via the kidneys and liver, they are not expected to significantly spread throughout the body for long time (Esparza et al., 2021). Upon ID administration of biotinylated VHH antibody to mice, and histopathology sampling, it was possible to detect it in the epidermis and dermis as well as in the blood, immediately upon administration. 3. VHH antibodies lack the Fc receptor and hence are not immunogenic. 4. Regulatory effort to reduce NHP use in preclinical research (Prior et al., 2017).

An in vitro tissue cross-reactivity (TCR) study may be used to identify non-specific and specific binding of the VHH antibodies in different types of human tissues. This information is vital in ensuring that experimental antibodies do not bind to epitopes other than the targeted sites, thus minimizing the risk of treatment-related toxicity.

An immunohistochemistry-based tissue cross-reactivity (TCR) screening assay is developed to identify the non-specific and specific binding of the VHH antibodies of the invention. This study is considered important to demonstrate that the tested moieties do not bind to epitopes other than the targeted sites. The establishment of the assay includes human tissues from 3 different donors.

As the primary goal is safety assessment, the intensity of the immunohistochemistry (IHC) staining can provide valuable insights into the potential toxicity. The IHC staining is investigated using a biotinylated (and/or FITC)-conjugated form of the VHH antibody on positive and negative control systems (target protein or BSA-spotted slides). The VHH antibody is tested on human tissues (3 donors) according to the EMA/FDA tissue list at 2 concentrations and the isotype control is tested in one concentration. After the experimental phase, pathology evaluation is performed. Tissue integrity is assessed using the von Willebrand factor as well as by two independent histopathologists.

To test histopathology (in standard organs) sub chronic administration to mini pigs may be performed for 28 days at 3 ascending doses.

To assess safety in humans, a phase I/IIa, randomized, double blind, placebo-controlled trial may be conducted. Tolerability, immunogenicity, pharmacokinetics, pharmacodynamics and efficacy of multiple ascending doses of anti-IL-17A/F VHH antibodies of the present invention are evaluated. The trial may be performed, for example in male and female subjects with mild to moderate psoriasis using intradermal and/or topical administration modes.

The phrase “subject in need thereof” used herein refers to a mammalian male or female subject (e.g., human being) who is diagnosed with an inflammatory disease or disorder. In a specific embodiment, this term encompasses individuals who are at risk to develop an inflammatory disease or disorder. The subject may be of any gender or at any age including neonatal, infant, juvenile, adolescent, adult and elderly adult.

As used herein the term “about” refers to ~10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

The term “about” refers to a value which is 10% above or below the indicated value.

It is noted that each possibility disclosed throughout the specification represents a separate embodiment of the present invention.

A non-limitative list of VHH antibodies of the present invention and their affinities and utility for IL-17 neutralization is shown in the following Table 3.

TABLE 3
Anti-IL-17A/F VHHs, their affinities (expressed as K_{D} values in pM), neutralization
potencies and thermostability.

| | | Affinity (pM)* | | Neutralization^{#} | | | Thermostability |
|---|---|---|---|---|---|---|---|
| VHHs | Class | IL-17A | IL-17F | IL-17A | IL-17F | IL-17AF | (° C.) |
| VHH662** | Control | 115 | 16000 | 2 | 4 | 1 | 76 |
| VHH664** | Control | 150 | 12000 | 2 | 4 | 2 | >95 |
| Re42B04a | A | 50 | 500 | 1 | 2 | 1 | >95 |
| Bm43B02 | A | 130 | 75 | 2 | 2 | 2 | 52 |
| Bm44B11 | A | 150 | 90 | 3 | 1 | 1 | 61 |
| Bm42B11 | A | ≤10 | 2000 | 1 | 2 | 1 | >95 |
| Bm44C09 | A | 2000 | 100 | 4 | 1 | 1 | 65 |
| Re42F08*** | B | 1000 | 6000 | 2 | 4 | 3 | >95 |
| Bm44B04*** | B | 600 | 350 | 2 | 2 | 1 | >95 |
| Bm44G07*** | B | 3000 | 1000 | 3 | 1 | 4 | >95 |
| Bm42A09*** | B | 800 | ≤10 | 2 | 1 | 1 | >95 |
| Bm42A03*** | C | 150 | 350 | 1 | 1 | 1 | 64 |
| Bm44G10*** | C | 200 | 350 | 1 | 3 | 1 | 63 |
| Bm45G07 | D | 60 | 4000 | 1 | 2 | 1 | >95 |

*Monovalent affinities as described in Example 3 and shown in FIGS. 4-8.
**These VHH antibodies of Ablynx were disclosed in WO2012156219.
***Two dissociation constants are calculated for VHHs whose BLI curves were fitted with a “2:1 heterogenous model”. Only high affinity component is reported in the table.
^{#}Neutralization potency was expressed as ranking, from 1 = the best to 4.
None of the VHH antibodies exhibited aggregation at 95° C., indicating their stability.

Comparison, using BLI, of the VHH antibodies of the present invention to known VHHs (for example those disclosed in WO2012156219), revealed that the VHH antibodies of the present invention binds with significantly higher affinity to IL-17F.

The challenge in the project was to get VHH antibodies that block IL-17 and bind tightly to both, IL-17A and IL-17F. This is not at all a trivial thing, because the neutralizing epitope has quite a few amino acid exchanges between IL-17A and IL-17F. Remarkably, binders with picomolar affinity to both IL-17A and IL-17F were identified, e.g., the VHH antibody denoted Bm43B02 and its Class A members.

The binding of some of the VHH antibodies of the present invention, in comparison to known VHH antibodies, is described in Table 3.

The affinities of the VHH antibody to the IL-17 isoforms may be measured using methods known in the art. Particular measurement methods use biolayer interferometry (BLI) instruments. According to some embodiments, biotinylated VHH antibodies are immobilized on the sensor chips and the binding of the homodimeric or heterodimeric IL17 species to them is measured. As the VHH antibodies are immobilized at a density that is not low, two neighboring VHH molecules can bind one IL-17 dimer at the same time. This makes an avidity effect, similar to a bivalent IgG binding to an IL-17 dimer. In another setup, IL-17 isoforms are immobilized on the chip and binding of monomeric VHH antibodies to them is measured, indicating more accurate KDs, as avidity effect due to dimeric nature of IL-17 is avoided. According to particular embodiments, the binding affinity of a monomeric VHH, expressed as a dissociation constant KD, to an immobilized human IL-17A or IL-17F homodimer is determined.

Crystal structures of VHH antibody-IL17 complexes (FIGS. 16 and 18 of Class A and Class B VHH antibodies respectively) document a deep understanding of the mode of action of the VHH antibodies. The structures and structure predictions were used to define (with near atomic resolution) the actual paratopes and to categorize the VHH antibodies in four classes.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Various embodiments and aspects of the present invention as delineated hereinabove and as claimed in the claims section below find experimental support in the following examples.

### EXAMPLES

### Example 1. Generation of VHH Antibodies

The generation of anti-IL-17 VHH antibodies showing a high-affinity cross-reaction between several different IL-17 isoforms was a highly challenging undertaking—given that the sequence identity between the IL-17 paralogues is limited to only ~50% identity between human IL-17A and IL-17F and that most conserved residues are actually buried in the hydrophobic core of the disulfide-bridged IL-17 dimer. The desired VHH antibody would thus likely have to tolerate sequences exchanges in its epitope without loss of binding strength. The IL-17 receptors can tolerate such sequence variability more easily because they have an interaction interface that is much larger than the epitope of a VHH antibody can possibly be.

The inventors, therefore, considered these constraints in their VHH generation strategy. They recombinantly produced the human IL-17A homodimer, the IL-17F homodimer, and the IL-17A/F heterodimer and injected the three multiple times as immunogens into an alpaca. This strategy was chosen to foster an immune response comprising also antibodies that cross-react between the IL-17 paralogues.

After the final immunization, a blood sample was taken, lymphocytes were isolated, and mRNA was purified and reverse transcribed. VHH-coding regions were amplified by nested PCR, reverse transcribed, and cloned as cDNAs into an M13 phagemid, yielding a library with >100 million independent clones.

In the next steps, several rounds of phage display were performed. Selected VHH antibodies were classified according to sequence similarity, cloned into E. coli expression vectors, produced by periplasmic expression, purified, and finally characterized for binding of IL-17 using biolayer interferometry (BLI; Abdiche et al., 2008).

In one arm of the selection approach, the IL-17A homodimer and the IL-17A/F heterodimer were used successively as baits. This yielded a considerable sequence diversity in the selected VHH antibodies-approximately 8 major classes and numerous minor ones as judged by sequence similarities. Most tested ones bound IL-17A with high affinities. ‘Binning’ identified three complementary epitopes (epitope 1-3), i.e., epitope 1-binding VHH antibodies could bind simultaneously with either epitope 2-binders or epitope 3-binders. Likewise, epitope 2- and 3-binders could also bind simultaneously. Epitope 2 and 3-binders, however, later turned out not to block IL-17 function.

In the second selection arm, the IL-17A homodimer, the IL-17F homodimer, and the IL-17A/F heterodimer were used successively as baits. This direct selection for cross-reactivity reduced the complexity of sequences considerably. All epitope 2- and epitope 3-binders were lost. Two VHH classes became dominant and accounted now for 98% of all sequences. These two classes are disclosed in the following and referred to as the Re42H11 and the Bm17B02 classes (Classes A and B as defined in Tables 1 and 2) and included Re42H11, Re42B03, Re42B04, Bm18B05, Bm18F11 (Class A) as well as Bm17B02, and Re42F08 (Class B).

For generation of a “second generation” VHH antibodies also to allow further affinity maturation of antibodies, 7 months after the last immunization, the alpaca was re-immunized with IL-17A and IL-17F proteins. Immune libraries were prepared, and phage display was performed, cross-selecting with alternating IL-17 versions as a bait, namely with IL-17A and IL-17F, with bait concentrations as low as 100 μM and including off-rate selection steps. This strategy yielded additional VHH antibodies (Tables 1, 2 and 3), including new VHH classes (e.g., Bm42A03 of Class C and Bm45G07 of Class D). However, most of the newly discovered VHH antibodies were variants of the previously isolated Re42H11 class (Class A) and Bm17B02 class (Class B) VHH antibodies.

### Example 2. Affinity Measurements Via VHH Immobilization

Initially, five members of the Re42H11 class (Re42H11 itself, Re42B03, Re42B04, Bm18B05, and Bm18F11), as well as two Bm17B02 class members (Bm17B02 and Re42F08), were characterized in depth. For affinity measurements, they were produced with a C-terminal Avi-Biotin-tag and enzymatically biotinylated by recombinant BirA (Beckett et al., 1999). They were subsequently immobilized at a concentration of 0.7 μg/ml for 110 seconds to High Precision Streptavidin biosensors on an Octet RED96e instrument (ForteBio/Sartorius), using Phosphate-Buffered Saline (PBS) pH 7.4, 0.02% (w/v) Tween 20 and 0.1% (w/v) bovine serum albumin (BSA) as an assay buffer. 10, 20, and 40 nM of indicated IL-17 species were then allowed to bind for 800 seconds and, subsequently, to dissociate for another 800 seconds. Binding and dissociation were recorded as wavelength shifts (in nm). Baselines were recorded by measuring a ‘minus VHH control’ in parallel. On-rates, off-rates, and dissociation constants (KDS) were calculated by the Octet Data Analysis HT 12.0 software), using a mass transport model to fit the data.

These measurements revealed perfect cross-reactivities with IL-17A, IL-17F, and IL17-A/F heterodimer (FIGS. 2 and 3), high on-rates (mostly around 10⁵-10⁶ M⁻¹·s⁻¹), non-detectable or nearly non-detectable dissociation rates, and apparent K_{D}s of 20 μM or better. Note that the BLI setup does not allow to discern affinities below 10 μM. Also note that this setup with immobilized VHHs includes avidity effects, whereby neighboring VHH molecules on the sensor chip can bind to the same IL-17 dimer, which contributes to the observed very low off-rates. This simulates the situation of a dimeric IgG binding to an IL-17 dimer.

### Example 3. Affinity Measurements Via IL-17 Immobilization

In order to measure the true monovalent affinities, the inventors also performed BLI via immobilizing the IL-17 species and used monovalent VHHs as the analyte. Results are shown in FIGS. 4-8 and 11, as well as in Table 3. In the first set of experiments, VHH662 and VHH664 from Ablynx/Sanofi were tested as control antibodies. They displayed high affinities for IL-17A with K_{D}s of 115 μM and 150 μM (for VHH662 and VHH664, respectively) but bound IL-17F poorly, with K_{D}s around 15 nM (FIG. 4).

When representatives of Class A and Class B VHH antibodies, namely Re42B04a and Re42F08, were probed for IL-17 binding under identical conditions, they showed better cross-reactivity, one example being Re42B04 with K_{D}s of 50 μM and 500 μM for IL-17A and IL-17F, respectively (FIG. 6). Several of second-generation anti-IL-17 VHHs showed remarkable cross-reaction between IL-17A and IL-17F, with affinities as high as 100 μM for both species (FIGS. 6, 7, and 8; Table 3).

### Example 4. Testing the Stability of the VHH Antibodies

For the intended therapeutic application, the IL-17 VHH antibodies should not only bind their targets with high affinity, but also, they should be developable as biological drugs. This includes that they are stable enough to survive a lengthy, large-scale production process as well as transportation and storage (ideally for years in formulation) without aggregation or loss of activity.

A good predictor for stability is thermostability, which can be measured, e.g., by thermal shift assays or specifically by differential scanning fluorimetry (Goldberg et al., 2011). The method exploits that melting (thermal unfolding) of a protein exposes aromatic/hydrophobic residues (from its hydrophobic core), which then bind and enhance the fluorescence of the added SYPRO Orange dye.

VHH antibodies were subjected to differential scanning fluorimetry (DSF). Assays were performed in a volume of 20 μl, at 1 mg/ml VHH concentration in 50 mM Tris/HCl, 150 mM NaCl (pH 8.0 at 20° C.), and 1×SYBR Organge dye (diluted from a 5000× stock; Life Technologies). The plate was sealed with transparent MicroSeal® ‘B’ Seal (Bio-Rad), briefly centrifuged to remove any air bubbles, and placed onto a CFX96 Real-Time System (C1000 Thermal Cycler, BioRad). The samples were incubated for 5 min at 20° C. The temperature was then increased by 1° C. every 45 seconds until 95° C. was reached. Fluorescence was measured at the end of each step with 532 nm excitation and a 555 nm long pass filter. Melting temperatures are defined as the inflection point of the first melting peak. If the melting peak remained lower than the initial fluorescence at 20° C., then this was interpreted as if no melting had occurred.

FIG. 9 shows such analyses for the disclosed VHH antibodies, with slow heating from 20° C. to 95° C. The majority of VHHs (Re42H11 itself, Re42B03, Re42B04, Re42B05, Bm18F11, as well as Re42F08) showed only a negligible unfolding signal and thus no melting (the small fluorescence peak did not or did hardly exceed the measured fluorescence at 20° C.). The very low melting amplitudes of Re42H11, Re42B03, Bm18F11, Bm18B05, and Re42F08 indicate resistance to melting and, thus, full thermal stability. As a control, the inventors repeated the experiment for some candidates in the presence of DTT (dithiothreitol) to reduce the structural disulfide bond and now, they observed large unfolding peaks that indicate a global melting of the respective VHH antibodies (FIG. 10). Note the far higher unfolding amplitude in the presence of DTT. These controls indicate which fluorescence signal to expect if a VHH antibody would melt completely.

One Re42H11 class member (Re42B04) showed a small melting peak that exceeded the 20° C.-signal and had an inflection point at 57° C. (FIG. 9); however, not even this VHH aggregated upon heating to 95° C. Thus, the here-disclosed anti-IL-17 VHH antibodies fulfill three key criteria for the intended application: extremely high affinity for their target, perfect cross-reaction between IL17A, IL17A/F, and IL17F, as well as extraordinarily high thermal stability.

The second generation VHH antibodies were also subjected to DSF as described in Example 2 and FIG. 4. All Bm17B02 (Class B) members (Bm44B04, Bm44G07, and Bm42A09), as well as Bm45G07 and Bm42B11 were resistant to melting even at 95° C., therefore they had full thermal stability (Table 3). Other VHH antibodies (Bm43B02, Bm44B11, Bm44C09, Bm42A03, and Bm44G10), on the other hand, showed melting between 50-60° C. These VHHs also did not aggregate upon heating to 95° C.

### Example 5. Testing VHH Stability by BLI

BLI is very sensitive to sample concentration and therefore suitable to detect any loss of active substance in a solution, including loss of active nanobody due to instability. To determine if the VHHs that melt already at 60° C. in DSF experiments preserve binding activity upon heat treatment, the antibodies were incubated at 95° C. for 10 minutes (at 1 μM concentration), cooled and then centrifuged to remove possibly formed aggregates. When they were tested for IL-17A binding by BLI, heated and untreated samples of Re42H11 and Bm42A03 class members (Classes A and C) showed no difference and behaved similar to the thermostable VHHs of Class A, namely Re42B04a and Bm42B11 (FIG. 11). Therefore, these nanobodies either melt only partially or robustly refold to their native states following heat treatment.

Note that both hyperthermostable VHHs (Re42B04a and Bm42B11) and VHHs that have a melting temperature of 50-60° C. (Bm43B02, Bm44B11, Bm44C09, Bm42A03, and Bm44G10) showed no difference upon heat treatment, indicating that any melting (unfolding) was reversible.

### Example 6. Cell-Based IL17 Stimulation Assay and Measurement of Reporter Activity

To monitor the biological activity of IL17 variants in a cell-based system, a HEK-Blue™ IL-17 reporter cell line (Invivogen hkb-il17) was used. These cells are HEK293 cells stably expressing the human IL-17RA and IL-17RC receptors as well as the adapter Act1/TRAF3IP2. HEK-Blue™ IL-17 cells further encode a secreted embryonic alkaline phosphatase (SEAP) reporter controlled by a promoter with NF-κB and AP-1 binding sites. Upon binding of the IL-17 ligands to its receptors on the cell surface, NF-kB and AP-1 get activated, SEAP is produced and secreted into the cell supernatant. The amount of SEAP can be measured colorimetrically, using QUANTI-Blue™ as a substrate.

Cells were cultivated in DMEM medium supplemented with 10% (v/v) fetal bovine serum, 4.5 g/L Glucose, 2 mM L-Glutamine, 100 U/mL penicillin, 100 μg/mL streptomycin and 100 μg/mL Normocin™. Selection antibiotics (HEK-Blue™ Selection) were introduced after the second passage and the growth medium was renewed at least twice per week. Cells were passaged at 70-80% confluency by scraping in PBS and not cultivated longer than 20 passages.

To evaluate the cellular response, titration curves were conducted with the different IL-17 variants. For this, HEK-Blue™ IL-17 cells were seeded in 96-well plates in medium without selection antibiotics and stimulated with serial three-fold dilutions of IL-17A (produced in HEK cells, Proteintech HZ-1113), IL-17F (produced in HEK cells, Proteintech HZ-1116), or IL-17A/F (produced in CHO cells, R&D Systems 5837-IL). Medium without cells and untreated cells served as negative controls. After overnight incubation, 20 μl cell supernatant from each well was mixed with 180 μl QUANTI-Blue™ solution in a separate 96-well plate and incubated for 30 min at 37° C. SEAP levels in the supernatant were determined by measuring the absorbance at 620 nm (OD620). The OD620 of the medium-only control served as the background and was subtracted from all sample wells. Each condition was assayed in triplicate. Induction was calculated from the ratio OD620 (sample)/OD620 (untreated). Based on the titration curves, the cells are more responsive to IL-17A (FIG. 12). Therefore, higher concentrations of IL-17F and IL-17A/F were used to achieve a comparable range of induction in the neutralization assays explained in Example 7.

### Example 7. Cell-Based IL17 Neutralization Assay

To assess if the disclosed anti-IL-17 VHH antibodies block the binding of IL-17 variants to their receptors, the inventors carried out cell-based neutralization assays using HEK-Blue™ IL-17 expressing cells. These cells express human IL-17RA and IL-17RC receptors as well as the adapter Act1. They secrete a phosphatase reporter in response to the receptor binding of IL-17 variants. By quantitating the amount of secreted phosphatase, the activation of the IL17 receptors can be measured as described in Example 6 (FIG. 12).

To test for neutralization, recombinant human IL-17A (5 ng/ml corresponding to 0.17 nM, Proteintech HZ-1113), IL-17F (25 ng/mL corresponding to 0.85 nM, Proteintech HZ-1116), or IL-17A/F (25 ng/ml corresponding to 0.85 nM, R&D Systems 5837-IL) were pre-incubated for two hours at 37° C. under constant shaking with serial three-fold dilutions of VHH antibodies before being added to the reporter cells. Medium without cells, untreated cells, and cells treated with IL-17 variants in the absence of any VHH served as controls. The stimulated cells were incubated over night at 37° C., 5% CO₂ and then the reporter activity was measured as described in Example 6. The OD620 of medium only served as the background and was subtracted from all sample wells. Each condition was assayed in triplicate. Normalized induction was calculated from the ratio OD620 (sample)/OD620 (IL17 only).

From the first generation, the following VHH antibodies tested were: the class A members Bm18B05, Bm18F11, Re42B03, Re42B04 and Re42H11 (IL-17A/IL-17F cross-reacting); the class B (Bm17B02) members Bm17B02 and Re42F08 (IL-17A/IL-17F cross-reacting); Bm17D12 (IL-17A preference); and Bm17B11 class members Bm17B11 and Bm18F01 (targeting the non-neutralizing epitope 3). The data show that a pre-incubation of IL-17A or IL-17F with any of the class A or B members impeded the receptor activation. Remarkably, IL-17A neutralization at sub-nanomolar concentrations was observed, e.g., for Re42B04, Re42B03, Bm17D12, and Re42F08. Likewise, the IL-17A/F heterodimer was neutralized potently whenever the VHHs Bm18F11, Re42B03, Re42B04, Re42H11, Bm17B02, Re42F08, or Bm17D12 exceeded the interleukin concentration. Re42H11, Re42B03, and Re42B04 were particularly efficient in blocking the IL-17F homodimer, with Re42H11 and Re42B03 showing a, essentially perfect stoichiometric neutralization. Bm17D12 is a control VHH that binds IL-17A tightly but IL-17F only weakly. Bm17B11 and Bm18F01 bind only IL-17A and this at the non-neutralizing epitope 3 (FIG. 13).

The second generation VHHs also blocked receptor activation when pre-incubated with IL-17A, IL-17F, or IL-17A/F (FIG. 14). Newly discovered VHH classes (Bm42A03 and Bm45G07) particularly showed excellent cross-neutralization, already at picomolar antibody concentrations (FIG. 14). In these assays, Re42B04a, Bm42A09, Bm42A03, and Bm45G07 as representative members of the VHH classes A-D outperformed VHH662 and VHH664 especially in terms of IL-17F neutralization (FIG. 15). Note the excellent neutralization at low nanomolar or even sub-nanomolar concentrations by members of all four VHH classes. For clarity, each graph lists the VHHs in an order of increasing neutralization potency.

The results shown in FIG. 13-15 indicate that the VHH antibodies completely neutralize IL-17A, IL-17F and IL-17AF at a concentration of <1 nM, <10 nM and <1 nM respectively.

### Example 8 Structural Characterization of IL-17•VHH Complexes

To understand the structural basis of the IL-17 inhibition, IL-17•VHH complexes were crystallized. The X-ray crystal structures of tetrameric IL-17F complexes of VHH antibodies representing two major VHH classes: Re42H11 and Bm17B02 (FIGS. 16 and 18) were solved. Note in that the IL-17RC receptor clashes with the VHH, explaining why the here disclosed class A anti-IL17 VHH antibodies block the binding of the interleukin to the receptor. Comparison of these structures with the receptor-bound IL-17F structure (IL1-17RC•IL-17F; Goepfert et al., 2020) revealed that Re42H11 and Bm17B02 block receptor-binding by directly competing for the IL-17-binding site.

To reveal the molecular details of the VHH-IL-17 interactions, homology models of all Re42H11 (Class A) and Bm17B02 (Class B) class members in complex with IL-17A and IL-17F were created. Analysis of these models allowed us to decipher contribution of individual amino acids for interleukin-binding as well as their influence on IL-17A/IL-17F cross-reaction. All VHH residues that are interacting with IL-17 species, IL17A and IL-17F, identified in the crystal or modelled structures, are listed in FIGS. 17 and 19. FIG. 17 positions in Class A VHH antibodies that interact with IL-17A or with IL-17F. FIG. 19 positions in Class B VHH antibodies that interact with IL-17A or with IL-17F.

The following positions in the sequences of Class A VHH antibodies were found to interact with IL-17A: position 1 that is Q; position 29 that is A, G V, F or P; position 30 that is S; position 31 that is S or G; position 32 that is Y; position 33 that is A; position 50 that is A; position 51 that is I; position 52 that is S; position 54 that is I, S, or V; position 55 that is S or G; position 57 that is G, S, or D; position 58 that is T, S or A; position 59 that is K, R or V; position 100 that is P; position 101 that is Y; position 103 that is L or M; position 104 that is D or E; position 106 that is R; and position 109 that is E or D.

The following positions in the sequences of Class A VHH antibodies were found to interact with IL-17F: Position 29 that is A, G V, F or P; position 30 that is S; position 31 that is S or G; position 32 that is Y; position 33 that is A; position 50 that is A; position 51 that is I; position 52 that is S; position 54 that is S, I or V; position 55 that is S or G; position 57 that is G, S, or D; position 58 that is T, S or A; position 59 that is K, R or V; position 100 that is P; position 101 that is Y; position 103 that is L or M; position 104 that is D or E; position 106 that is R; and position 109 that is E or D.

The following positions in the sequences of Class B VHH antibodies were found to interact with IL-17A: position 3 that is Q, position 31 that is I or Q, position 32 that is S, position 33 that is A, position 37 that is Y, position 45 that is R, position 52 that is H, position 59 that is H or Y, position 99 that is N, position 100 that is E, position 101 that is P, position 102 that is G, position 103 that is H or D, position 104 that is L, position 105 that is Y and position 106 that is M.

The following positions in the sequences of Class B VHH antibodies were found to interact with IL-17F: position 3 that is Q, position 31 that is I or Q, position 32 that is S, position 33 that is A, position 37 that is Y, position 45 that is R, position 47 that is L, position 50 that is L or M, position 52 that is T or H, position 59 that is H or Y, position 99 that is N, position 100 that is E, position 101 that is P, position 102 that is G, position 103 that is H or D, position 104 that is L, position 105 that is Y and position 106 that is M.

Differences in interaction are due to differing residues in IL-17A and IL-17F. The information was used to identify critical residues of VHHs. When classifying nanobodies, we can look at the conservation of the interacting residues rather than their complete sequence. For Bm17B02 class for example, although new nanobodies have different CDR1 and CDR2 sequences and hence one might argue that they are distinct classes, by looking at the interacting residues, we can see that they are almost identical.

### Example 9. Production of the VHH Antibodies in Pichia pastoris

VHH antibodies Re42B04a and Re42F08 were produced recombinantly in Pichia pastoris. FIGS. 20A and 20B describe the upstream and downstream process respectively.

As described in the schemes of FIG. 20A, the upstream stage was initiated by culturing an inoculum from the cell bank, followed by fermentation, induction of expression by adding methanol and harvest. The downstream stages presented in FIG. 20B included adjustment of pH, when needed, and purification by passing the filtrated harvest over several columns followed by 20 mM phosphate buffer formulation.

### Example 10. Ex-Vivo Human Skin Model

InflammaSkin® is a psoriasis-like model developed by Genoskin to reproduce the key features of pro-Th17/Th1 inflammation associated with psoriasis. The model, presented schematically in

FIG. 21, relies on in-situ activation of resident T-cell of normal skin biopsies and their further polarization into Th17/Th1 phenotype with the supplementation of a culture medium containing a cocktail of pro-inflammatory cytokines. This model has been successfully validated with topically applied compounds, including steroids, delivered either in a prophylactic or therapeutic manner.

The model was employed to evaluate the effective dose, and the dosing regimen prior to conducting in-vivo studies in mice transplanted with psoriatic human skin. The VHH antibodies Re42B04a and Re42F08, were compared to monoclonal antibody targeting IL-17A (i.e., Secukinumab) used clinically as subcutaneously injection for treatment of psoriasis, and to steroidal treatment with betamethasone.

Skin samples from female donors, with no record of current inflammatory skin disease or treatment are cultured under standard cell culture conditions (CO₂ incubator at 37° C., water saturation, daily medium renewal). Next, in situ activation and Th17/Th1 polarization of skin resident T cells with pro-inflammatory cytokine cocktail is added to induce the psoriatic phenotype, followed by ID treatment with VHH antibody, once on day 3 or thrice on days 3-5. Positive control groups include standard care of topical betamethasone, or daily prophylactic treatment or single SC administration of Secukinumab on day 3, while negative control treatments include irrelevant VHH antibody Re32D03 that does not bind IL-17.

The anti-inflammatory effect was evaluated by measuring cytokine secretion, including IL-17 family cytokines, interferon 1β (IFN-1β) and interferon Y (IFN-γ). Additionally, skin's structure, integrity & viability are assessed by a histological analysis (e.g., H&E staining). The study design is shown in Table 4.

TABLE 4
Ex vivo study design

| | | | | | | | | Sampling/Analysis | | |
|---|---|---|---|---|---|---|---|---|---|---|
| | | Treatment | | | | | | timepoints | | |
| | | | | | | Start | End | Day | | Day |
| # | Model | Treatment | Dose | Route | Frequency | date | date | 0 | Day 3 | 7 |
| 1 | HypoSkin | Control | NA | | | | | (n = 1) | (n = 1) | n = 1 |
| | | Untreated | | | | | | | | |
| 2 | Hypo- | Control | NA | | | | | (n = 1) | (n = 5) | n = 5 |
| | InflammaSkin | Untreated | | | | | | | | |
| 3 | | Positive control | 0.05% | Topical 25 | Daily | Day 0 | Day 6 | — | (n = 3) | n = 3 |
| | | Betamethasone | | μL | | | | | | |
| 4 | | Secukinumab | 160 μg | Subcutaneous | Once | Day 3 | — | — | (n = 3) | n = 3 |
| | | | | 35 μL | | | | | | |
| 5 | | Irrelevant VHH | 11 | Intradermal | Once | Day 3 | — | — | (n = 3) | n = 3 |
| 6 | | VHH | mg/mL | 35 μL | Once | Day 3 | — | — | (n = 3) | n = 3 |
| | | Re42B04a | | | | | | | | |
| 7 | | VHH Re42B04a | | | Day 3, 4, 5 | Day 3 | Day 5 | — | (n = 3) | n = 3 |
| 8 | | VHH | | | Once | Day 3 | — | — | (n = 3) | n = 3 |
| | | Re42F08 | | | | | | | | |
| 9 | | VHH | | | Day 3, 4, 5 | Day 3 | Day 5 | — | (n = 3) | n = 3 |
| | | Re42F08 | | | | | | | | |

| | |
|---|---|
| Total number of HypoSkin ® models | n = 1 |
| Total number of Hypo-InflammaSkin model | n = 26 |

Culture volume was 2 mL.

IL-17A, appeared as the most significantly affected cytokine by the applied treatments (FIG. 22). As expected, low levels of IL-17A were detected in culture media of the untreated HypoSkin model (around 7 pg/ml was measured) while a very pronounced secretion was detected in the untreated HypoInflammaSkin models, reaching on average 130 pg/ml and confirming an efficient Th17 type response. Importantly both the positive control treatment, Betamethasone and Secukinumab, efficiently and significantly inhibited IL-17A secretion. The control, irrelevant VHH Re32D03 (anti SARS-COV-2), did not significantly affect the IL-17A secretion, however more variability between the replicates was observed when compared to other conditions (varying from 39 to 135 pg/ml). VHH Re42B04a injected once and three times almost completely abrogated IL-17A secretion to 6.8 and 5.7 pg/ml, respectively with one replicate for each condition below detection limit). This was approximately a 20 fold reduction when compared to HypoInflammaSkin condition and around 14 fold when compared to the irrelevant VHH. VHH Re42F08 injected once and thrice also significantly reduces IL-17A expression, however to a lesser extent than Re42B04a, resulting in average concentrations of 40 and 48 pg/ml, respectively.

Hematoxylin & Eosin (H&E) staining was performed on day 7 to evaluate the structural integrity and cellular viability of the skin samples at this time point. Basic commentary in the following areas were evaluated: Cellular apoptosis and loss of cellular viability of the epidermis: this is typically indicated by pyknotic nuclei (dark, condensed) and/or hypereosinophilic cytoplasm (stained “pink” instead of purple) in the epidermis. Broken adipocytes in the subcutaneous layer, could be indicative of cellular viability loss. Spongiosis: Fluid infiltration into epidermis. This typically appears as “white” spaces between the keratinocytes with the interconnecting filaments still visible. There may also be smaller white pockets/spaces, especially near the epidermal/dermal junction. Structural Integrity Loss: Separation of the epidermis and dermis, often associated with large areas of pyknosis in the basal epidermal layer.

As shown in FIGS. 23A-23H: Untreated normal tissue control (FIG. 23A) vs Untreated psoriasis-induced tissue (FIG. 23B) showed Loss of cellular viability, large regions of pyknotic and hypereosinophilic cells, and increased epidermal thickness. With the betamethasone (FIG. 23C) and Secukinumab (FIG. 23D) treatments there was improved viability, but some signs of inflammation are still apparent. When treated with the VHH antibodies, Re42b04a injected once (FIG. 23E) or thrice (FIG. 23F), or Re42F08 injected once (FIG. 23G) or thrice (FIG. 23H) pathological changes were significantly prevented and skin histology was improved compared to untreated psoriasis-induced tissue (FIG. 23B). In comparison to multiple cells with pyknosis in the untreated control, and a loss of structural integrity, these phenomena are limited in the samples treated with Secukinumab and the two VHHs. In FIGS. 24E and 24G that were treated once with the VHHs, the tissue looks integral with no occurrence of Psoriatic damage.

### CONCLUSIONS

The objective of this study was to evaluate the anti-inflammatory effects of VHH antibodies of the present invention using the InflammaSkin platform in which T-cell activation and stimulation leads to Th1/Th17-mediated induction of psoriasis in the skin. The VHH antibodies Re42B04a and Re42F08, were injected intradermally into HypoInflammaSkin models once or three times after the onset of inflammation. An irrelevant VHH directed against the spike protein of SARS-CoV-2 was used as a control for this experimental group and was injected once. Betamethasone, a topical steroid, was used as a positive control treatment. Additionally, Secukinumab a commercialized anti-IL-17A antibody used clinically to treat psoriasis, was injected intradermally to compare its anti-inflammatory potential with the tested VHH antibodies. All models were cultivated for seven days before accessing the effects of the applied treatments.

To assess the effect of the positive control, betamethasone treatment, on donor's responsiveness to T cell activation, release of the cytokine IL-22 into the culture media was evaluated after 7 days. Additionally, H&E staining of HypoSkin and HypoInflammaSkin sectioned samples was used to analyze skin structural integrity and cellular viability. Finally, cytokine release using MSD TH17 Combo 2 assay (K15076K, MesoScale Discovery), was evaluated in all conditions on Day 7.

Overall, no significant changes in skin features were observed during the course of culture and H&E staining was indicative of healthy and viable HypoSkin untreated models. Upon inflammation induction, the HypoInflammaSkin models showed severe loss of cellular viability, hyperkeratosis, presence of spongiosis indicative of ongoing inflammation and signs of aberrations in structural integrity. Betamethasone, the positive control treatment, appeared to improve skin histology especially when assessing the extent of pyknosis and stratum corneum thickening. Secukinumab treatment seemed to improve skin features even more, with less pronounced evidence of cellular death and hyperkeratosis. The irrelevant VHH did not impact the characteristic features of HypoInflammaSkin models and the extent of microscopic changes was comparable to the untreated HypoinflammaSkin group. The tested VHH antibodies, Re42B04a and Re42F08 significantly improved skin histology. HypoInflammaSkin models injected once with VHH antibodies Re42b04a and Re42f08showed significantly less areas of pyknosis, hypereosinophilic keratinocytes and no striking hyperkeratosis was observed in contrast to the control conditions. Spongiosis persisted in those experimental conditions and basal epidermis still showed signs of decreased cellular viability. Altogether, the H&E results demonstrated an improvement in skin histology by the tested VHH antibodies Re42B04a and Re42F08, which appeared more efficient than betamethasone to reduce inflammation induced skin changes in this donor and at least as efficient as Secukinumab.

The most significant and striking results were obtained for IL-17A secretion analysis. Its expression was significantly up-regulated in response to T-cell activation and stimulation. Both topical treatment with betamethasone and injection of Secukinumab significantly reduced its levels. VHH antibody Re42B04a (injected once and thrice) reduced IL-17A secretion to its basal levels detected in the untreated HypoSkin control, resulting in a stronger effect when compared to betamethasone or Secukinumab. VHH antibody Re42F08 (injected once and thrice) also significantly reduced IL-17A release, but to a lesser extent than Re42B04a, betamethasone and Secukinumab. Importantly, when compared to the irrelevant VHH treatment group, the decrease in IL-17A secretion was significant for VHH Re42F08 (both treatment schemes).

Overall, obtained results demonstrated a strong potential of the VHH antibodies of the present invention to modulate inflammation in InflammaSkin model of psoriasis as indicated by improving skin viability and structural integrity. This was corroborated by cytokine release analysis, especially IL-17A expression.

### REFERENCES

Abdiche Y, Malashock D, Pinkerton A, Pons J (2008) Determining kinetics and affinities of protein interactions using a parallel real-time label-free biosensor, the Octet. Anal Biochem, 377:209-217
Beckett D, Kovaleva E, Schatz PJ (1999) A minimal peptide substrate in biotin holoenzyme synthetase-catalyzed biotinylation. Protein Sci, 8:921-929
Su, S.; Esparza, T. J.; Nguyen, D.; Mastrogiacomo, S.; Kim, J. H.; Brody, D. L. Pharmacokinetics of Single Domain Antibodies and Conjugated Nanoparticles Using a Hybrid near Infrared Method. Int. J. Mol. Sci. 2021, 22, 8695
Glatt S, Baeten D, Baker T, Griffiths M, Ionescu L, Lawson A D G, Maroof A, Oliver R, Popa S, Strimenopoulou F, Vajjah P, Watling MIL, Yeremenko N, Miossec P, Shaw S (2018) Dual IL-17A and IL-17F neutralisation by bimekizumab in psoriatic arthritis: evidence from preclinical experiments and a randomised placebo-controlled clinical trial that IL-17F contributes to human chronic tissue inflammation. Ann Rheum Dis, 77:523-532
Goepfert, A., Lehmann, S., Blank, J., Kolbinger, F., & Rondeau, J. M. (2020). Structural Analysis Reveals that the Cytokine IL-17F Forms a Homodimeric Complex with Receptor IL-17RC to Drive IL-17RA-Independent Signaling. Immunity, 52 (3), 499-512.e5
Goldberg D S, Bishop S M, Shah A U, Sathish H A (2011) Formulation development of therapeutic monoclonal antibodies using high-throughput fluorescence and static light scattering techniques: role of conformational and colloidal stability. J Pharm Sci, 100:1306-1315
Güttler T, Aksu M, Dickmanns A, Stegmann K M, Gregor K, Rees R, Taxer W, Rymarenko O, Schünemann J, Dienemann C, Gunkel P, Mussil B, Krull J, Teichmann U, Groß U, Cordes VC, Dobbelstein M, Görlich D (2021) Neutralization of SARS-COV-2 by highly potent, hyperthermostable, and mutation-tolerant nanobodies. EMBO J, e107985
Keren A, Shemer A, Ginzburg A, Ullmann Y, Schrum A G, Paus R, Gilhar A. Innate lymphoid cells 3 induce psoriasis in xenotransplanted healthy human skin. J Allergy Clin Immunol. 2018 July; 142 (1): 305-308
Frank Kolbinger, Franco Di Padova, Atul Deodhar, Jason E. Hawkes, Christine Huppertz, Torsten Kuiper, Iain B. McInnes, Christopher T. Ritchlin, David Rosmarin, Georg Schett, José M. Carballido, Peter Häusermann, Claudio Calonder, Beate Vogel, Jean-Michel Rondeau, Gerard Bruin, Secukinumab for the treatment of psoriasis, psoriatic arthritis, and axial spondyloarthritis: Physical and pharmacological properties underlie the observed clinical efficacy and safety, Pharmacology & Therapeutics, Volume 229, 2022, 107925,
Langley R G, Elewski B E, Lebwohl M, Reich K, Griffiths C E, Papp K, Puig L, Nakagawa H, Spelman L, Sigurgeirsson B, Rivas E, Tsai T F, Wasel N, Tyring S, Salko T, Hampele I, Notter M, Karpov A, Helou S, Papavassilis C, ERASURE SG, FIXTURE SG (2014) Secukinumab in plaque psoriasis—results of two phase 3 trials. N Engl J Med, 371:326-338
Mease P J, McInnes I B, Kirkham B, Kavanaugh A, Rahman P, van der Heijde D, Landewé R, Nash P, Pricop L, Yuan J, Richards H B, Mpofu S, FUTURE SG (2015) Secukinumab Inhibition of Interleukin-17A in Patients with Psoriatic Arthritis. N Engl J Med, 373:1329-1339
Mills K H G (2022) IL-17 and IL-17-producing cells in protection versus pathology. Nat Rev Immunol, 1-17
Nies J F, Panzer U (2020) IL-17C/IL-17R E: Emergence of a Unique Axis in TH17 Biology. Front Immunol, 11:341
Papp K A, Weinberg M A, Morris A, Reich K (2021) IL17A/F nanobody sonelokimab in patients with plaque psoriasis: a multicentre, randomised, placebo-controlled, phase 2b study. Lancet, 397:1564-1575
Helen Prior, Fiona Sewell, Jane Stewart,
Overview of 3Rs opportunities in drug discovery and development using non-human primates, Drug Discovery Today: Disease Models, Volume 23, 2017, Pages 11-16,
Reich K, Warren R B, Lebwohl M, Gooderham M, Strober B, Langley R G, Paul C, De Cuyper D, Vanvoorden V, Madden C, Cioffi C, Peterson L, Blauvelt A (2021) Bimekizumab versus Secukinumab in Plaque Psoriasis. NEngl J Med, 385:142-152
Sarama et al., Frontiers in Bioengineering and Biotechnology February 2022|Volume 10|Article 803218.
Skroza N, Proietti I, Bernardini N, Aquila E, Balduzzi V, La Viola G, Mambrin A, Muscianese M, Tolino E, Zuber S, Potenza C, Ferrazza P (2017) Il-17 and Its Role in Psoriasis, Hidradenitis Suppurativa And Acne. Internal Medicine and Care, 1 (2): 1-6
Su, S.; Esparza, T. J.; Nguyen, D.; Mastrogiacomo, S.; Kim, J. H.; Brody, D. L. Pharmacokinetics of Single Domain Antibodies and Conjugated Nanoparticles Using a Hybrid near Infrared Method. Int. J. Mol. Sci. 2021, 22, 8695.
Warren R B, Blauvelt A, Bagel J, Papp K A, Yamauchi P, Armstrong A, Langley R G, Vanvoorden V, De Cuyper D, Cioffi C, Peterson L, Cross N, Reich K (2021) Bimekizumab versus Adalimumab in Plaque Psoriasis. N Engl J Med, 385:130-141
Wilson S C, Caveney N A, Yen M, Pollmann C, Xiang X, Jude K M, Hafer M, Tsutsumi N, Piehler J, Garcia K C (2022) Organizing structural principles of the IL-17 ligand-receptor axis. Nature, 609:622-629
Zhang Y. Huo M. Zhou J. Xie S. PKSolver: an add-in program for pharmacokinetic and pharmacodynamic data analysis in Microsoft Excel. Comput Methods Programs Biomed. 2010; 99:306-314
Zhou, X. et al. Advances in the pathogenesis of psoriasis: from keratinocyte perspective. 2022; Cell Death Dis 13, 81

1-50. (canceled)

51. A VHH antibody recognizing a human IL-17 polypeptide, which cross-reacts with a plurality of different human IL-17 polypeptides comprising (i) a human IL-17A homodimer, (ii) a human IL-17F homodimer and (iii) a human IL-17A/F heterodimer, and neutralizes IL-17 receptor activation, the VHH antibody comprising a CDR3 sequence selected from
i. a sequence of the formula NDMPYGX₁X₂TX₃MDX₄YX₅X₆W wherein, X₁ is selected from L and M, X₂ is selected from D and E, X₃ is selected from R and T, X₄ is selected from E and D, X₅ is selected from A, V, E D and K and X₆ is selected from Y and S (class A);
ii. a sequence of the formula X₁HNEPGX₂LYM wherein, X₁ is selected from V and T and X₂ is selected from H and D (class B);
iii, the sequence MAVRGLYGSNWYDYPFELW (SEQ ID NO. 52), (class C); and
iv. the sequence YIDSGSDRYY (SEQ ID NO. 57), (class D).
v.

52. The VHH antibody of claim 51 selected from:
i. a class A VHH antibody comprising a sequence selected from SEQ ID NOs. 19, 15, 5, 23, 12, 13, 20, 27, 28, and 32 or a variant thereof having at least 90% identity;
ii. a class B VHH antibody comprising a sequence selected from SEQ ID NOs. 39, 35, 40, 44, and 45 or a variant thereof having at least 90% identity;
iii. a class C VHH antibody comprising a sequence selected from SEQ ID NOs. 49, and 53 or a variant thereof having at least 90% identity; and
iv. a class D VHH antibody comprising the sequence selected from SEQ ID NO. 54 or a variant thereof having at least 90% identity.

53. The VHH antibody of claim 51, comprising an amino acid sequence selected from SEQ ID 19, 15, 5, 39, 23, 12, 13, 20, 27, 28, 32, 35, 40, 44, 45, 49, 53, and 54.

54. The class A VHH antibody of claim 51, comprising a set of 3 CDR sequences, the set is selected from: SEQ ID NOs. 16, 17 and 18; SEQ ID NOs. 6, 7 and 8; SEQ ID NOs. 10, 7 and 11; SEQ ID NOs. 10, 14 and 11; SEQ ID NOs. 21, 22 and 18; SEQ ID NOs. 24, 25 and 26; SEQ ID NOs. 29, 30 and 31; SEQ ID NOs. 33, 7 and 34.

55. The class A VHH antibody of claim 54, comprising the following sequence positions that interact with IL-17A: position 1 that is Q; position 29 that is A, G V, F or P; position 30 that is S; position 31 that is S or G; position 32 that is Y; position 33 that is A; position 50 that is A; position 51 that is I; position 52 that is S; position 54 that is I, S, or V; position 55 that is S or G; position 57 that is G, S, or D; position 58 that is T, S or A; position 59 that is K, R or V; position 100 that is P; position 101 that is Y; position 103 that is L or M; position 104 that is D or E; position 106 that is R; and position 109 that is E or D, and/or the following sequence positions that interact with IL-17F: position 1 that is Q; position 29 that is A, G V, F or P; position 30 that is S; position 31 that is S or G; position 32 that is Y; position 33 that is A; position 50 that is A; position 51 that is I; position 52 that is S; position 54 that is I, S, or V; position 55 that is S or G; position 57 that is G, S, or D; position 58 that is T, S or A; position 59 that is K, R or V; position 100 that is P; position 101 that is Y; position 103 that is L or M; position 104 that is D or E; position 106 that is R; and position 109 that is E or D.

56. The class B VHH antibody of claim 51, comprising a set of 3 CDR sequences, the set is selected from: SEQ ID NOs. 36, 37, and 38; SEQ ID NOs. 41, 42 and 43; and SEQ ID NO. 46, 42 and 43.

57. The class B VHH antibody of claim 56, comprising the following sequence positions that interact with IL-17A: position 3 that is Q, position 31 that is I or Q, position 32 that is S, position 33 that is A, position 37 that is Y, position 45 that is R, position 52 that is H, position 59 that is H or Y, position 99 that is N, position 100 that is E, position 101 that is P, position 102 that is G, position 103 that is H or D, position 104 that is L, position 105 that is Y and position 106 that is M, and/or the following sequence positions that interact with IL-17F: position 3 that is Q, position 31 that is I or Q, position 32 that is S, position 33 that is A, position 37 that is Y, position 45 that is R, position 47 that is L, position 50 that is L or M, position 52 that is T or H, position 59 that is H or Y, position 99 that is N, position 100 that is E, position 101 that is P, position 102 that is G, position 103 that is H or D, position 104 that is L, position 105 that is Y and position 106 that is M.

58. The VHH antibody according to claim 51, selected from RE42B04a (SEQ ID NO. 19), Re42B04 (SEQ ID NO. 15), Re42F08 (SEQ ID NO. 39), Bm43B02 (SEQ ID NO. 23), and Bm17B02 (SEQ ID NO. 35), or a VHH antibody, which is a variant thereof having at least 90% identity to any of these sequences.

59. The VHH antibody according to claim 51 comprising a set of 3 CDR sequences, the set comprises SEQ ID NOs. 50, 51 and 52 (class C), or SEQ ID NOs. 56, 57 and 58 (class D).

60. The VHH antibody according to claim 51, having in a monomeric form, a binding affinity expressed as a dissociation constant KD, to an immobilized human IL-17A or IL-17F homodimer of 5 nM, 1 nM, 500 pM, 300 pM, 100 pM, 50 pM, or less, and/or that neutralizes the binding of at least one of (i) a human IL-17A homodimer, (ii) a human IL-17F homodimer and (iii) a human IL17A/F heterodimer to a human IL-17 receptor, at a concentration of about 10 nM or less, of about 3 nM or less, of about 1 nM or less, of about 0.3 nM or less, when tested in a cell-based assay under affinity-limited test conditions.

61. The VHH antibody according claim 51, which is thermostable or hyperthermostable having a melting temperature of at least about 65° C., of at least about 80° C., of at least 90° C. or of at least about 95° C. when measured under non-reducing conditions and/or an aggregation temperature of at least about 60° C., of at least 70° C., of at least about 80° C., of at least about 90° C., or of at least about 95° C., when measured under non-reducing conditions.

62. The VHH antibody according to claim 51, which is conjugated or fused to a heterologous moiety.

63. A set of two or more different VHH antibodies recognizing a human IL-17 polypeptide, particularly an IL-17A homodimer, an IL-17F homodimer and an IL17A/F heterodimer, comprising at least one VHH antibody according to claim 51.

64. A nucleic acid molecule encoding a VHH antibody according to claim 51, a vector comprising said nucleic acid molecule, or a recombinant cell or a non-human organism transformed or transfected with said nucleic acid molecule or vector.

65. A method for recombinant production of a VHH antibody according to claim 51, comprising cultivating cells or an organism in a suitable medium and obtaining the VHH antibody from the cells or organism or from the medium.

66. A pharmaceutical composition comprising a VHH antibody according to claim 51, and a pharmaceutically acceptable excipient, or diluent.

67. A diagnostic composition comprising a VHH antibody according to claim 51, and an acceptable excipient, or diluent.

68. A method for the prevention or treatment of a disease or disorder caused by and/or associated with an overactivity of IL-17, particularly an overactivity of IL-17A and/or IL-17F, comprising administering the pharmaceutical composition of claim 66 to a subject in need thereof.

69. The method of claim 68, wherein the disease or disorder is an inflammatory and/or immune-related disorder or an infectious disease.

70. The method of claim 69, wherein the disease or disorder is a skin disorder or an autoimmune disease selected from psoriasis particularly plaque psoriasis, moderate to severe psoriasis, hypertrophic palmoplantar psoriasis, pustular psoriasis, or pustular psoriasis, arthritis particularly rheumatoid arthritis, psoriatic arthritis, ankylosing spondylitis, or enthesitis-related arthritis, systemic lupus erythematosus (SLE), familial mediterranean fever, and inflammatory bowel disease (IBD).
